SEQUENCE LISTING (4 sequences)

SEQ ID NO: 1 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 218 aa, synthetic construct
DIQMTQSPSSLSASVGDRVTITCRASQSVSTSSYNYMHWYQQKPGKPPKLLIKYASNLESGVPSRFSGSGSGTDFTLTISSLQPEDFATYYCQHSWEIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 452 aa, synthetic construct
EVQLVQSGAEVKKPGASVKVSCKASGYSFTDYYMHWVRQAPGQGLEWIGRINPYAGAAFYSQNFKDRVTLTVDTSTSTAYLELSSLRSEDTAVYYCAIERGADLEGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0007761 A1
(43) Pub. Date: Jan. 8, 2026

(54) Bioactive Conjugate, Preparation Method Therefor and Use Thereof

(71) Applicant: BeOne Medicines I GmbH, Basel (CH)

(72) Inventors: Charng-Sheng Tsai, Beijing (CN); Mei-Hsuan Tsai, Shanghai (CN); Bing Li, Shanghai (CN); Liming Wu, Shanghai (CN)

(73) Assignee: BeOne Medicines I GmbH, Basel (CH)

(21) Appl. No.: 19/332,995

(22) Filed: Sep. 18, 2025

Related U.S. Application Data
(63) Continuation of application No. PCT/IB2024/052794, filed on Mar. 22, 2024.

(30) Foreign Application Priority Data
Mar. 23, 2023 (WO) PCTCN2023083522

(51) Int. Cl. A61K 47/68; C07D 285/08; C07D 417/04

(52) U.S. Cl. CPC A61K 47/6803; C07D 285/08; C07D 417/04

(57) ABSTRACT

The present disclosure provides antibody drug conjugate platforms comprising a conjugator assembly component, and antibody drug conjugates comprising platform-derived linker-payloads and antibodies or antigen-binding fragments thereof. In some embodiments, an antibody drug conjugate is of the following formula

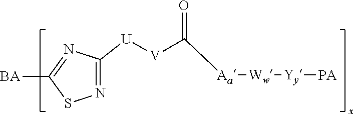

or a pharmaceutically acceptable salt, tautomer, solvate, or stereoisomer thereof, wherein values for the variables (e.g., BA, U, V, A, a′, W, w′, Y, y′, PA, x) are as described herein.

### 1. CROSS REFERENCE TO RELATED APPLICATIONS

This application is a continuation of International Application No. PCT/IB2024/052794, filed Mar. 22, 2024, which claims priority to International Application No. PCT/CN2023/083522, filed Mar. 23, 2023, the disclosures of each of which are hereby incorporated by reference in their entireties.

### 2. SEQUENCE LISTING

The instant application contains a Sequence Listing, which has been submitted electronically in XML format and is hereby incorporated by reference in its entirety. Said XML copy, created on Sep. 18, 2025, is named “01368-0070-00US”, and is 5,842 bytes in size.

### 3. FIELD

Provided herein are antibody drug conjugate platforms and antibody drug conjugates (ADCs) comprising the platforms plus an antibody, or antigen-binding fragment thereof, as well as uses of the ADC platforms and ADCs.

### 4. BACKGROUND

Antibody drug conjugates (ADCs) combine the targeting effect of antibodies with the cell-killing activity of bioactive molecules, making them “biological missiles.” An ADC is guided by an antibody to bind target cells, and then is internalized by cells to release bioactive molecule payloads, thereby treating relevant diseases. The bioactive molecule is covalently coupled to the antibody via a linker.

Lysine is the most common linking site in antibodies, and F-amino groups thereof can react with activated carboxyl groups of linkers to form amide bonds. Techniques for site-specific coupling are currently available, that is, carboxyl groups of linkers are activated and then form amide bonds with specific lysine F-amino groups in antibodies to complete the coupling. However, such amide bonds are prone to hydrolysis under the action of enzymes in vivo. As a result, bioactive molecules and antibodies dissociate before reaching target cells, resulting in off-target toxicity.

Thiol groups of antibody cysteine residues usually exist in the form of disulfide bonds. The disulfide bonds in the antibody can be broken to provide multiple free sulfhydryl groups as coupling sites. One method of coupling with the antibody sulfhydryl groups is a Michael addition reaction between the free sulfhydryl groups and an electrophilic maleimide functional group, or two Michael addition reactions between a specific substrate and free sulfhydryl groups of the antibody to form a sulfur bridge bond in a unique structure. WO2016142049 discloses amatoxins as bioactive molecules, and structures comprising amatoxins bound to methylsulfonyl-substituted oxadiazole-based linkers, but details of coupling with antibodies are not specifically described. As has been noted by multiple investigators in the bioconjugate field, the thiol-substituted product of the reaction between the electrophilic maleimide functional group and a free thiol of an antibody is subject to slow elimination, thus reversing the reaction.

When this reversible reaction occurs in a purified preparation of an ADC, the reaction is largely undetectable because the maleimide and thiol that are regenerated through the elimination process simply react again, thus reforming the intact conjugate. However, when other thiols are present, the net effect can be the transfer of the maleimide from the antibody of the ADC onto any other available thiol. This process has been documented to occur in plasma, in which the maleimide of an ADC transfers to cysteine 34 of serum albumin (Alley et al., Bioconjugate Chem. 2008, 19, 759-765). This process has also been reported when an ADC is incubated in the presence of excess cysteine or glutathione (Jununtula et al., Nature Biotech, 2012). The present disclosure is directed to, inter alia, bioconjugates that do not undergo this transfer reaction.

### 5. BRIEF SUMMARY

Provided herein are antibody drug conjugate platforms and antibody drug conjugates (ADCs). Also provided are uses of the ADC platforms to prepare ADCs.

In some embodiments, provided herein are ADC compounds of Formula (I):

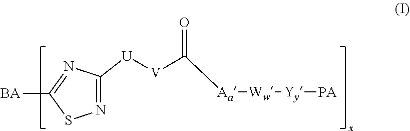

or a pharmaceutically acceptable salt, tautomer, solvate, or stereoisomer thereof, wherein:
BA is a binding agent selected from a humanized, chimeric, or human antibody or an antigen binding fragment thereof,
U is arylene, heteroarylene, or a bond;
V is a bond or —C≡C—(CH₂)ₙ—;
n is an integer between 0 and 10;
A is a Stretcher unit residue;
subscript a′ is 0 or 1;
W is a Cleavable unit;
subscript w′ is 0 or 1;
Y is a Spacer unit;
subscript y′ is 0 or 1;
PA is a payload residue; and
subscript x is from 1 to 15.

In some embodiments, the platform is linker-payload compound of Formula (II):

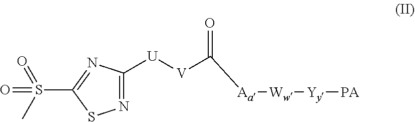

or a pharmaceutically acceptable salt, tautomer, solvate, or stereoisomer thereof, wherein:
U is arylene, heteroarylene, or a bond;
V is a bond or —C≡C—(CH₂)ₙ—;
n is an integer between 0 and 10;
A is a Stretcher unit residue;
subscript a′ is 0 or 1;
W is a Cleavable unit;
subscript w′ is 0 or 1;
Y is a Spacer unit;
subscript y′ is 0 or 1; and
PA is a payload residue.

In some embodiments, the platform is a linker compound of Formula (III):

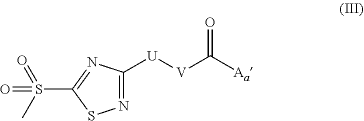

or a pharmaceutically acceptable salt, tautomer, solvate, or stereoisomer thereof, wherein:
U is arylene, heteroarylene, or a bond;
V is a bond or —C≡C—(CH₂)ₙ—;
n is an integer between 0 and 10;
A is a Stretcher unit; and
subscript a′ is 0 or 1.

Additional objects and advantages will be set forth in part in the description that follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate embodiments and together with the description serve to explain the principles described herein.

### 6. BRIEF DESCRIPTION OF FIGURES

FIG. 1 depicts stability data of conjugator-antibody conjugate 3-1 in pH 7.4 and 8.0 buffer with and without GSH.

FIG. 2 depicts stability data of conjugator-antibody conjugate 3-2 in pH 7.4 and 8.0 buffer with and without GSH.

FIG. 3 depicts stability data of conjugator-antibody conjugate 3-3 in pH 7.4 and 8.0 buffer with and without GSH.

FIG. 4 depicts stability data of conjugator-antibody conjugate 3-4 in pH 7.4 and 8.0 buffer with and without GSH.

FIG. 5 depicts stability data of conjugator-antibody conjugate 3-5 in pH 7.4 and 8.0 buffer with and without GSH.

FIG. 6 depicts stability data of conjugator-antibody conjugate 3-1 in pH 5.5 histidine buffer for 168 h.

FIG. 7 depicts stability data of conjugator-antibody conjugate 3-2 in pH 5.5 histidine buffer for 168 h.

FIG. 8 depicts stability data of conjugator-antibody conjugate 3-3 in pH 5.5 histidine buffer for 168 h.

FIG. 9 depicts stability data of conjugator-antibody conjugate 3-4 in pH 5.5 histidine buffer for 168 h.

FIG. 10 depicts stability data of conjugator-antibody conjugate 3-5 in pH 5.5 histidine buffer for 168 h.

FIG. 11 depicts stability data of ADC 4-1 in pH 7.4 or 8.0 GSH buffer.

FIG. 12 depicts stability data of ADC 4-2 in pH 7.4 or 8.0 GSH buffer.

FIG. 13 depicts stability data of ADC 4-3 in pH 7.4 or 8.0 GSH buffer.

FIG. 14 depicts stability data of ADC 4-1 in formulation buffer.

FIG. 15 depicts stability data of ADC 4-2 in formulation buffer.

FIG. 16 depicts stability data of ADC 4-3 in formulation buffer.

FIG. 17 depicts ADC direct killing activity on HL60 cells.

FIG. 18 depicts ADC direct killing activity on U937 cells.

FIG. 19 depicts ADC direct killing activity on TF1 cells.

FIG. 20 depicts ADC direct killing activity on a B7H3 high-expression cell line (H1650).

FIG. 21 depicts ADC direct killing activity on a B7H3 low-expression cell line (Capan-1).

FIG. 22 depicts ADC direct killing activity on a B7H3(−) cell line (MB-453).

FIG. 23 depicts ADC direct killing activity on a B7H3 high-expression cell line (H1650).

FIG. 24 depicts ADC direct killing activity on a B7H3 low-expression cell line (Capan-1).

FIG. 25 depicts ADC direct killing activity on a B7H3(−) cell line (MB-453).

FIG. 26 depicts the ADC bystander killing on NCI-H358/MDA-MB-453 (nano-Luc) in a co-culture assay.

FIG. 27 depicts the ADC bystander killing on NCI-H358/MDA-MB-453 (nano-Luc) in a co-culture assay.

FIG. 28 depicts the ADC bystander killing on NCI-H358/MDA-MB-453 (nano-Luc) in a co-culture assay.

FIG. 29 depicts the ADC bystander killing on NCI-H358/MDA-MB-453 (nano-Luc) in a co-culture assay.

FIG. 30 is a line graph showing ADC anti-tumor activity in an H1650 xenograft model.

FIG. 31 is a line graph showing dose-dependent ADC anti-tumor activity in an H1650 xenograft model.

FIG. 32 is a line graph showing payload release rate from ADCs in mouse plasma.

FIG. 33 is a line graph showing payload release rate from ADCs in human plasma.

FIG. 34 is a line graph showing payload release rate from ADCs in mouse plasma.

FIG. 35 is a line graph showing payload release rate from ADCs in human plasma.

FIG. 36 is a line graph showing the PK profiles of ADCs in tumor-bearing mice.

FIG. 37 is a line graph showing the PK profiles of ADCs in non-tumor-bearing mice.

### 7. DETAILED DESCRIPTION

Provided herein are antibody drug conjugates (ADCs) and covalent linkers and linker-payloads (platforms) for making ADCs. The ADCs may be used to treat a disease or disorder, such as cancer, such as by providing a composition comprising an ADC. The presently disclosed ADCs are more stable than known ADCs.

Some conjugators are known to be difficult to conjugate with binding agents, such as requiring harsh reaction conditions that can detrimentally modify the binding agents, or requiring multiple reaction steps. The presently disclosed conjugator assemblies are efficiently conjugated to cysteine residues on antibodies without the need for, e.g., a separate hydrolysis step. Some ADCs are known to undergo deconjugation under physiological conditions. The presently disclosed conjugator-antibody conjugates are more resistant to premature deconjugation in plasma due to improved coupling of the disclosed conjugator assemblies to binding agents and payloads.

### 7.1. Definitions

In the present disclosure, the following terms have the following meanings unless indicated otherwise. Unless defined otherwise, all technical and scientific terms used herein have the same meaning as is commonly understood by one of ordinary skill in the art to which this disclosure pertains. In the event that there is a plurality of definitions for a term provided herein, these Definitions prevail unless stated otherwise.

The term “antibody” herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. An intact antibody has primarily two regions: a variable region and a constant region. The variable region binds to and interacts with a target antigen. The variable region includes a complementary determining region (CDR) that recognizes and binds to a specific binding site on a particular antigen. The constant region may be recognized by and interact with the immune system (see, e.g., Janeway et al., 2001, Immuno. Biology, 5th Ed., Garland Publishing, New York). An antibody can be of any type (e.g., IgG, IgE, IgM, IgD, and IgA), class (e.g., IgG1, IgG2, IgG3, IgG4, IgA1, and IgA2) or subclass. The antibody can be derived from any suitable species. In some embodiments, the antibody is of human or murine origin. An antibody can be, for example, human, humanized, or chimeric.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” is not to be construed as requiring production of the antibody by any particular method.

An “intact antibody” is one that comprises an antigen-binding variable region as well as a light chain constant domain (CL) and heavy chain constant domains, CH1, CH2, CH3, and CH4, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragment of any of the above which immunospecifically binds to a target antigen (e.g., a cancer cell antigen, a viral antigen or a microbial antigen).

An “antigen” is an entity to which an antibody specifically binds.

The terms “specific binding” and “specifically binds” mean that the antibody or antibody derivative will bind, in a highly selective manner, to its corresponding target antigen and not with the multitude of other antigens. Typically, the antibody or antibody derivative binds with an affinity of at least about 1×10⁻⁷ M, 10⁻⁸ M, 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

The term “inhibit” or “inhibition of” means to reduce by a measurable amount, or to prevent entirely.

The term “therapeutically effective amount” refers to an amount of a drug effective to treat a disease or disorder in a mammal. In the case of cancer, the therapeutically effective amount of a drug may reduce the number of cancer cells; reduce the tumor size; inhibit (i.e., slow to some extent or stop) cancer cell infiltration into peripheral organs; inhibit (i.e., slow to some extent or stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “substantial” or “substantially” refers to a majority, i.e. >50% of a population, of a mixture or a sample, such as more than 50%, 55%, 60%, 65%, 70%, 75%, 80%, 85%, 90%, 91%, 92%, 93%, 94%, 95%, 96%, 97%, 98%, or 99% of a population.

The terms “intracellularly cleaved” and “intracellular cleavage” refer to a metabolic process or reaction inside a cell on a ligand drug conjugate (e.g., an antibody drug conjugate (ADC)), whereby the covalent attachment, e.g., the linker, between the drug moiety (D) and the ligand unit (e.g., an antibody (BA or Ab)) is broken, resulting in the free drug, or another metabolite of the conjugate dissociated from the antibody inside the cell. The cleaved moieties of the drug-linker-ligand conjugate are thus intracellular metabolites.

The term “cytotoxic activity” refers to a cell-killing, a cytostatic or an anti-proliferative effect of a drug-linker-ligand conjugate compound or an intracellular metabolite of a drug-linker-ligand conjugate. Cytotoxic activity may be expressed as the IC50 value, which is the concentration (molar or mass) per unit volume at which half the cells survive.

The term “cytotoxic agent” as used herein refers to a substance that inhibits the function of cells and/or causes destruction of cells. The term is intended to include radioactive isotopes (e.g., 211At, 131I, 125I, 90Y, 186Re, 188Re, 153Sm, 212Bi, 32P, 60C, and radioactive isotopes of Lu), chemotherapeutic agents, and toxins such as small molecule toxins or enzymatically active toxins of bacterial, fungal, plant, or animal origin, including synthetic analogs and derivatives thereof.

The terms “cancer” and “cancerous” refer to or describe the physiological condition or disorder in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer); lung cancer including small-cell lung cancer, non-small cell lung cancer (“NSCLC”), adenocarcinoma of the lung, and squamous carcinoma of the lung; cancer of the peritoneum; hepatocellular cancer; gastric or stomach cancer including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; as well as head and neck cancer.

An “autoimmune disease” herein is a disease or disorder arising from and directed against an individual's own tissues or proteins.

Examples of a “patient” include, but are not limited to, mammals such as a human, rat, mouse, guinea pig, monkey, pig, goat, cow, horse, dog, or cat, and birds or fowl. In an embodiment, the patient is a human.

The terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of inhibiting growth of tumor cells, cancer cells, or of a tumor, inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of inhibiting replication of cells associated with an autoimmune disease state including, but not limited to, cells that produce an autoimmune antibody, lessening the autoimmune-antibody burden, and ameliorating one or more symptoms of an autoimmune disease.

As used herein, and in the specification and the accompanying claims, the indefinite articles “a” and “an” and the definite article “the” include the plural as well as single referents, unless the context clearly indicates otherwise.

As used herein, and unless otherwise specified, the terms “about” and “approximately,” when used in connection with amounts, or weight percentage of ingredients of a composition, mean an amount or weight percent that is recognized by one of ordinary skill in the art to provide a pharmacological effect equivalent to that obtained from the specified amount or weight percent. In certain embodiments, the terms “about” and “approximately,” when used in this context, contemplate an amount or weight percent within 30%, within 20%, within 15%, within 10%, or within 5%, of the specified amount or weight percent.

As used herein, and unless otherwise specified, the terms “about” and “approximately,” when used in connection with a numeric value or range of values that is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4% 3% 2%, 1.5%, 1%, 0.5%, or 0.25% of the recited value or range of values. For example, in some embodiments, the value of an XRPD peak position may vary by up to ±0.2° 20 while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. Representative alkyl groups include -methyl, -ethyl, -n-propyl, -n-butyl, -n-pentyl, and n-hexyl; saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), C(CH₂CH₃)═CH₂, C≡CH, —C≡C(CH₃), —C≡C(CH₂CH₃), —CH₂C≡CH, —CH₂C≡C(CH₃), and CH₂C≡C(CH₂CH₃), among others. An alkyl group can be substituted or unsubstituted. In certain embodiments, when the alkyl groups described herein are said to be “substituted,” they may be substituted with any substituent or substituents as those found in the compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂; or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3-octenyl and the like. The double bond of an alkenyl group can be unconjugated or conjugated to another unsaturated group. An alkenyl group can be unsubstituted or substituted.

A “cycloalkyl” group is a saturated or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, and hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6 to 14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

An “arylene” group is a bivalent aryl group as defined herein.

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heteroarylene” group is a bivalent heteroaryl group as defined herein.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl, and imidazolidinyl groups. The term “heterocyclyl” includes fused ring species, including those comprising fused aromatic and non-aromatic groups, such as, for example, benzotriazolyl, 2,3-dihydrobenzo[1,4]dioxinyl, and benzo[1,3]dioxolyl. The term also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula -alkyl-aryl, wherein alkyl and aryl are defined above. Substituted aralkyl groups may be substituted at the alkyl, the aryl, or both the alkyl and the aryl portions of the group. Representative aralkyl groups include, but are not limited to, benzyl and phenethyl groups and fused (cycloalkylaryl)alkyl groups such as 4-ethyl-indanyl.

A “heterocyclylalkyl” group is a radical of the formula -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include, but are not limited to, 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl)O(alkyl), wherein alkyl is defined above.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂-4 alkynyl. Examples of alkynyl moieties include, but are not limited to, ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

An “amino” group is a radical of the formula NH₂.

A “hydroxyl amine” group is a radical of the formula N(R¹⁹⁰)OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula —N(R^{#})O-alkyl or —NHO-alkyl, wherein R^{#} is as defined above.

An “aralkoxyamine” group is a radical of the formula N(R^{#})O-aryl or NHOaryl, wherein R^{#} is as defined above.

An “alkylamine” group is a radical of the formula NHalkyl or N(alkyl)₂, wherein each alkyl is independently as defined above.

An “aminocarbonyl” group is a radical of the formula —C(═O)N(R^{#})₂, —C(═O)NH(R^{#}), or C(═O)NH₂, wherein each R^{#} is as defined above.

An “acylamino” group is a radical of the formula NHC(═O)(R^{#}) or N(alkyl)C(═O)(R^{#}), wherein each alkyl and R^{#} are independently as defined above.

An “O(alkyl)aminocarbonyl” group is a radical of the formula —O(alkyl)C(═O)N(R^{#})₂, —O(alkyl)C(═O)NH(R^{#}), or —O(alkyl)C(═O)NH₂, wherein each R^{#} is independently as defined above.

An “N-oxide” group is a radical of the formula —N⁺—O⁻.

A “carboxy” group is a radical of the formula C(═O)OH.

A “ketone” group is a radical of the formula C(═O)(R^{#}), wherein R^{#} is as defined above.

An “aldehyde” group is a radical of the formula —CH(═O).

An “ester” group is a radical of the formula C(═O)O(R^{#}) or OC(═O)(R^{#}), wherein R^{#} is as defined above.

A “urea” group is a radical of the formula —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula —N═C(R^{#})₂ or —C(R^{#})═N(R^{#}), wherein each R^{#} is independently as defined above.

An “imide” group is a radical of the formula —C(═O)N(R^{#})C(═O)(R^{#}) or N((C═O)(R⁴))₂, wherein each R^{#} is independently as defined above.

A “urethane” group is a radical of the formula —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R⁴)₂)₂, —N═C(NH(R^{#}))₂, or —N═C(NH₂)₂, wherein each R^{#} is independently as defined above.

An “enamine” group is a radical of the formula —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or —C(R⁴)═C(R^{#})(NH₂), wherein each R^{#} is independently as defined above.

An “oxime” group is a radical of the formula —C(═NO(R^{#}))(R^{#}), —C(═NOH)(R^{#}), —CH(═NO(R^{h})), or —CH(═NOH), wherein each R^{#} is independently as defined above.

A “hydrazide” group is a radical of the formula —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula —N(R^{#})N(R^{#})₂, —NHN(R⁴)₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula —N₃.

An “isocyanate” group is a radical of the formula N═C═O.

An “isothiocyanate” group is a radical of the formula N═C═S.

A “cyanate” group is a radical of the formula OCN.

A “thiocyanate” group is a radical of the formula SCN.

A “thioether” group is a radical of the formula —S(R^{#}), wherein R^{#} is as defined above.

A “thiocarbonyl” group is a radical of the formula —C(═S)(R^{#}), wherein R^{#} is as defined above.

A “sulfinyl” group is a radical of the formula —S(═O)(R^{#}), wherein R^{#} is as defined above.

A “sulfone” group is a radical of the formula —S(═O)₂(R^{#}), wherein R^{#} is as defined above.

A “sulfonylamino” group is a radical of the formula —NHSO₂(R^{#}) or —N(alkyl)SO₂(R^{#}), wherein each alkyl and R^{#} are defined above.

A “sulfonamide” group is a radical of the formula —S(═O)₂N(R^{#})₂, —S(═O)₂NH(R^{#}), or —S(═O)₂NH₂, wherein each R^{#} is independently as defined above.

A “phosphonate” group is a radical of the formula —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula —P(R^{#})₂, wherein each R⁴ is independently as defined above.

When the groups described herein, with the exception of alkyl groups, are said to be “substituted,” they may be substituted with any appropriate substituent or substituents. Illustrative examples of substituents are those found in the compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid or base and an organic acid or base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters may be formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted that the compounds can include E and Z isomers, or a mixture thereof, and cis and trans isomers, or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

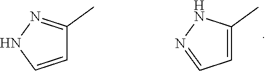

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (3H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight It should be noted that if there is a discrepancy between a depicted structure and a name for that structure, the depicted structure is to be accorded more weight.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, but not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

Certain groups, moieties, substituents, and atoms are depicted with a wiggly line, e.g., that intersects a bond or bonds, to indicate the atom through which the groups, moieties, substituents, atoms are bonded. For example, a phenyl group that is substituted with a propyl group depicted as:

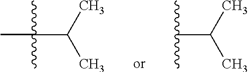

has the following structure:

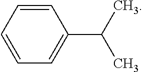

Illustrations showing substituents bonded to a non-cyclic group through a bond between two atoms are meant to indicate, unless specified otherwise, that the substituent may be bonded to either atom of the bond through which the substituent bond passes, according to techniques set forth herein or which are known in the field to which the instant disclosure pertains. Thus, for example,

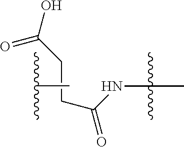

encompasses

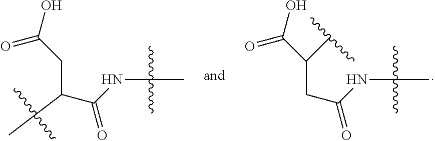 [SMILES: *NC(=O)C(*)CC(=O)O ; *NC(=O)CC(*)C(=O)O]

As used herein, “binding agent” refers to any molecule, e.g., antibody, capable of binding with specificity to a given binding partner, e.g., antigen.

As used herein, the term “amino acid” refers to an organic compound that contains amino (—NH₂) and carboxyl (—COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine, and citrulline.

As used herein “peptide”, in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including glycine and both the D and L optical isomers, and amino acid analogs and peptidomimetics. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include post-expression modifications of the polypeptide, for example, glycosylation, acetylation, phosphorylation, and the like. Furthermore, as ionizable amino and carboxyl groups are present in the molecule, a particular peptide may be obtained as an acidic or basic salt, or in neutral form. A peptide may be obtained directly from the source organism or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

As used herein, the term “anti-HER2 antibody” refers to an antibody selectively binding to the HER2 receptor, e.g., trastuzumab (Herceptin). In one embodiment, trastuzumab can be made and used as described in U.S. Pat. Nos. 6,407,213 and 5,821,337, the entire disclosures of which are incorporated herein by reference.

As used herein, the term “ifinatamab” refers to an antibody selectively binding to the B7H3 receptor. In one embodiment, ifinatamab can be made and used as described in US110117952 or WO2022102695, the entire disclosures of which are incorporated herein by reference.

As used herein, the term “cell-killing activity” refers to the activity that decreases or reduces the cell viability of the tested cell line.

In the claims that follow and in the preceding description, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments.

### 7.2. Conjugates

In embodiments, a conjugate, or a pharmaceutically acceptable salt, tautomer, solvate, or stereoisomer thereof, includes a protein linked to at least one payload or payload residue (also referred to herein as a Drug unit) and linked to at least one hydrophilic moiety via a covalent linker. The covalent linker is bonded directly or indirectly to each of the protein, the payload residue, and the hydrophilic moiety. In some embodiments, the protein is a binding agent, such as an antibody or antigen binding fragment thereof.

In some embodiments, the protein is bonded directly to a covalent linker, such as a linker set forth herein. In such cases, the binding agent is one bond position away from the covalent linker. The covalent linker may also be bonded directly to a payload residue such that the covalent linker is one bond position away from a payload residue. The payload may be any payload set forth herein. In some embodiments, the covalent linker is also bonded directly to a hydrophilic moiety such that the covalent linker is one bond position away from a hydrophilic moiety. The hydrophilic moiety may be any hydrophilic moiety (HG) set forth herein.

In some embodiments, the binding agent is bonded indirectly to a covalent linker such that the binding agent is more than one bond position away from the covalent linker. In such cases, the binding agent is bonded through another moiety to the covalent linker. For example, the binding agent may be bonded to a maleimide group which is bonded to a polyethylene glycol group which is bonded to the covalent linker.

In some examples, the covalent linker is also bonded indirectly to a payload residue such that the covalent linker is more than one bond position away from a payload residue. The covalent linker is bonded through another moiety to the payload. For example, the covalent linker may be bonded to a dipeptide, such as but not limited to Val-Ala or Val-Cit, which may be bonded to PAB which may be bonded to the payload residue.

In some embodiments, the covalent linker is bonded indirectly to a hydrophilic moiety such that the covalent linker is more than one bond position away from a hydrophilic moiety. The covalent linker is bonded through another moiety to the hydrophilic moiety.

### 7.2.1. Aspect 1

Provided herein are ADCs, e.g., for use in therapy, such as cancer therapy

One embodiment is an ADC compound of Formula (I):

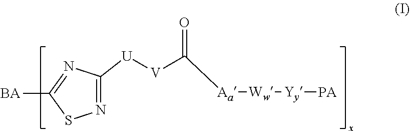

or a pharmaceutically acceptable salt, tautomer, solvate, or stereoisomer thereof, wherein:
BA is a binding agent selected from a humanized, chimeric, or human antibody or an antigen binding fragment thereof,
U is arylene, heteroarylene, or a bond;
V is a bond or —C≡C—(CH₂)ₙ—;
n is an integer between 0 and 10;
A is a Stretcher unit residue;
subscript a′ is 0 or 1;
W is a Cleavable unit;
subscript w′ is 0 or 1;
Y is a Spacer unit;
subscript y′ is 0 or 1;
PA is a payload residue; and
subscript x is from 1 to 15.

In one embodiment, subscript x is from 1 to 15. In one embodiment, subscript x is from 1 to 12. In one embodiment, subscript x is from 1 to 10. In one embodiment, subscript x is from 2 to 10. In one embodiment, subscript x is from 3 to 10. In one embodiment, subscript x is from 4 to 10. In one embodiment, subscript x is from 4 to 9. In one embodiment, subscript x is from 4 to 8.

In some embodiments, U and V are not each a bond at the same time.

In one embodiment, U is a bond. In one embodiment, U is arylene. In one embodiment, U is phenylene. In some embodiments, U is

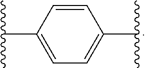 [SMILES: *c1ccc(*)cc1]

In one embodiment, U is heteroarylene. In one embodiment, U is a bivalent pyrimidine ring. In one embodiment, U is

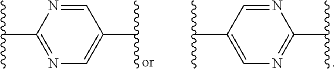 [SMILES: *c1cnc(*)nc1 ; *c1cnc(*)nc1]

In one embodiment, U is

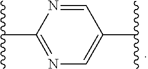 [SMILES: *c1cnc(*)nc1]

In one embodiment, U is

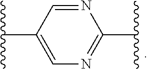 [SMILES: *c1cnc(*)nc1]

In one embodiment, V is a bond. In one embodiment, V is —C≡C—(CH₂)ₙ—. In one embodiment, V is —C≡C—(CH₂)₃—.

In some embodiments, A is —(CH₂)ₘ—C(═O)—, —CH₂—C(═O)—NH—(CH₂)ₘ—C(═O)—, —(CH₂CH₂O)ₘ—CH₂CH₂—C(═O)—, —CH[—(CH₂)ₘ—COOH]—C(═O)—, —CH₂—C(═O)—NH—(CH₂)ₘ—C(═O)—NH—(CH₂)ₘ—C(═O)—, —C(═O)—(CH₂)ₘ—C(═O)—, —NH—(CH₂)ₘ—C(═O)—, or —NH—(CH₂CH₂O)ₘ—CH₂CH₂—C(═O)—, wherein each m independently represents an integer of 1, 2, 3, 4, or 5.

In some embodiments, W is one of the following formulas:

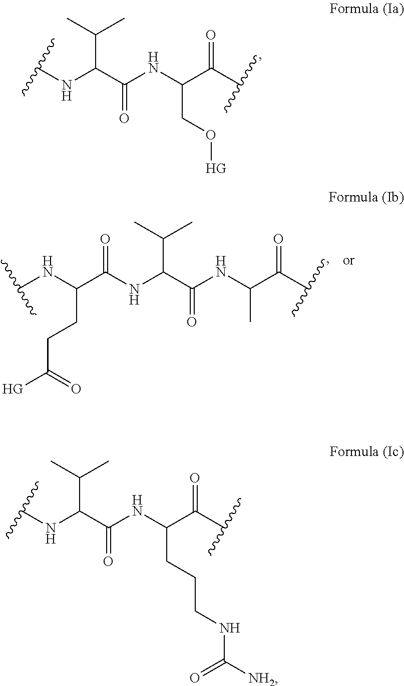

wherein HG is a hydrophilic moiety or hydrogen.

In some embodiments, HG is a saccharide, phosphate ester, sulfate ester, phosphodiester, or phosphonate.

In some embodiments, HG is a saccharide selected from β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid or any analogue or modification thereof, or sulfate, phosphate, carboxyl, amino, or O-acetyl modification thereof.

In some embodiments, HG is

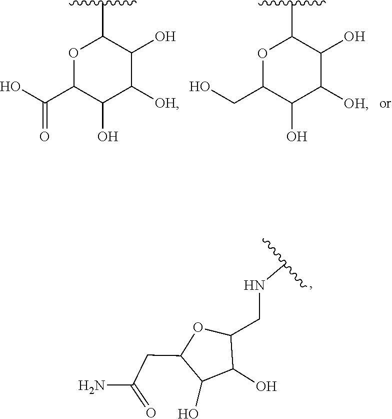 [SMILES: *C1OC(C(=O)O)C(O)C(O)C1O ; *C1OC(CO)C(O)C(O)C1O ; "or" ; *NCC1OC(CC(N)=O)C(O)C1O]

or a stereoisomer thereof. In some embodiments, HG is

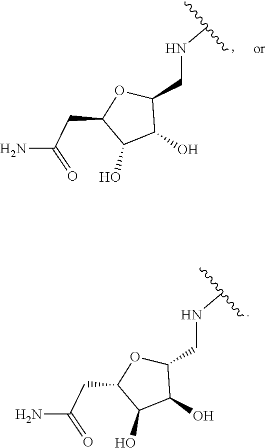 [SMILES: *NC[C@@H]1O[C@H](CC(N)=O)[C@@H](O)[C@H]1O ; *NC[C@H]1O[C@@H](CC(N)=O)[C@H](O)[C@@H]1O]

In some embodiments, Y is —NH—CH₂—O— or —NH-(p-C₆H₄)—CH₂—O—.

In some embodiments, the ADC compound of Formula (I) has one of the following formulas:

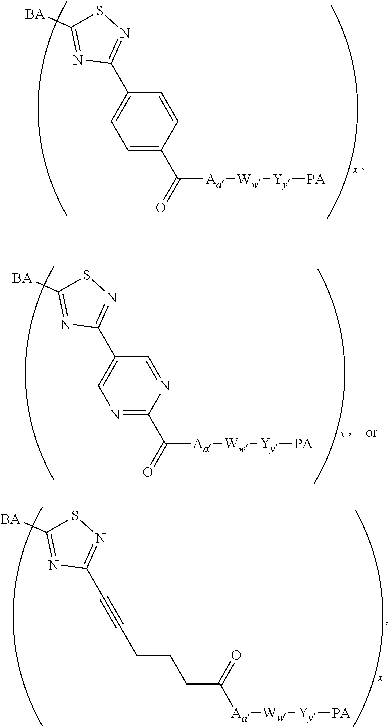

wherein values for the variables (e.g., BA, PA, A, W, Y, a′, w′, y′, x) are as described above.

In some embodiments, the fraction

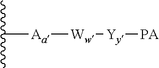

is one of the following formulas:

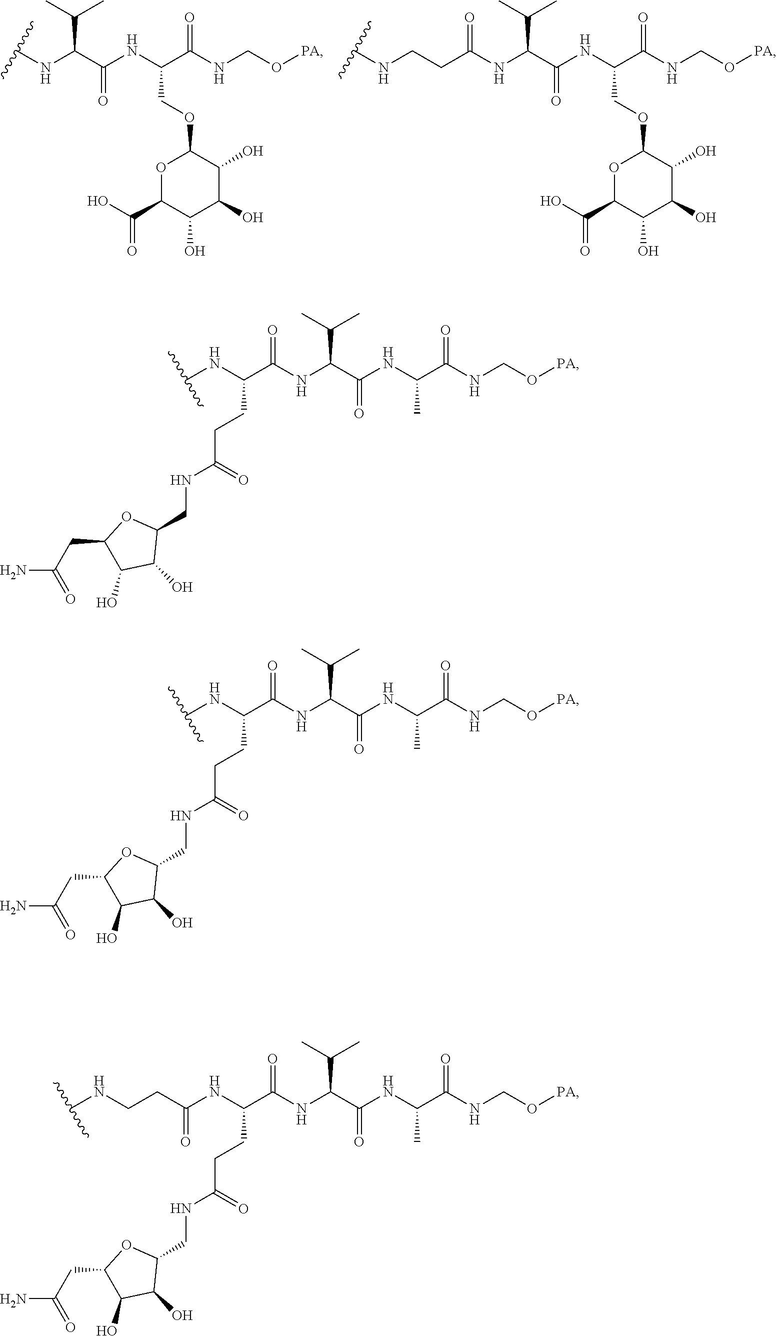

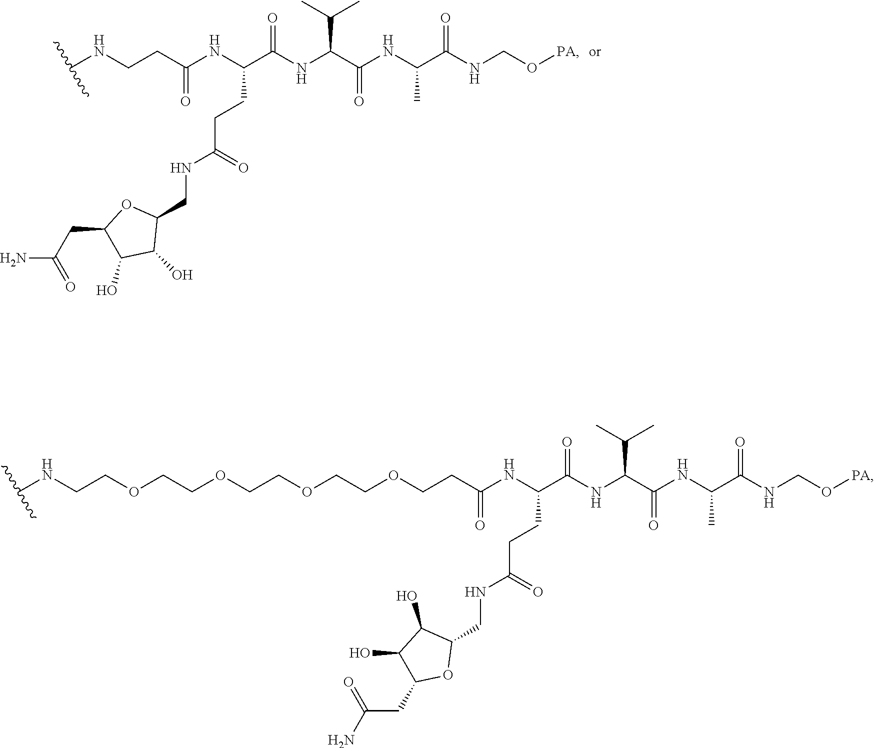

wherein PA is as described below.

In some embodiments, the Stretcher unit (-A-) is present and extends the framework of the covalent linker to provide more distance between a conjugator assembly and a Drug unit (payload or payload residue). The conjugator assembly may include the components of Formula (I) other than the binding agent (BA), Stretcher unit (A), Cleavable unit (W), Spacer unit (Y), and payload residue (PA). The conjugator assembly may include the components of Formula (II) other than the Stretcher unit (A), Cleavable unit (W), Spacer unit (Y), and payload residue (PA). The conjugator assembly may include the components of Formula (III) other than the Stretcher unit (A). In embodiments, the conjugator includes the methylsulfonyl group, thiadiazole group, U, V, and carbonyl group of Formulas (I), (II), and/or (III). A Stretcher unit may link the conjugator assembly to the Cleavable unit when the Cleavable unit is present, the conjugator assembly to the Spacer unit when the Cleavable unit is absent and the Spacer unit is present, or the conjugator assembly to the Drug unit when both the Cleavable unit and the Spacer unit are absent. A Stretcher unit may attach to more than one Cleavable unit, Spacer unit, and/or Drug unit. The conjugator assembly, Cleavable unit, Spacer unit, and Drug unit may be any conjugator assembly, Cleavable unit, Spacer unit, and Drug unit, respectively, described herein.

The Stretcher unit may alter the physiochemical properties of the Drug-Linker depending on the components of the Stretcher unit. In some implementations, the Stretcher unit may increase the solubility of the Drug-Linker and may include one or more solubility-enhancing groups such as ionic groups or water-soluble polymers. Water-soluble polymers may be soluble in water at room temperature and may include poly(ethylene)glycol groups as well as other polymers such as polyethyleneimines.

A Stretcher unit may comprise one or more stretcher groups. Examples of stretcher groups include, for example, —C₁₋₁₀ alkylene-C(O)—, —C₁₋₁₀ alkylene-C(O)—NH—C₁₋₁₀ alkylene-C(O)—, —(CH₂CH₂O)ᵤ—CH₂CH₂—C(O)—, —CH[—C₁₋₁₀ alkylene-COOH]—C(O)—, —C₁₋₁₀ alkylene-C(O)—NH—C₁₋₁₀ alkylene-C(O)—NH—C₁₋₁₀ alkylene-C(O)—, —C(O)—C₁₋₁₀ alkylene-C(O)—, —NH—C₁₋₁₀ alkylene-C(═O)—, —NH—(CH₂CH₂O)ᵤ—CH₂CH₂—C(═O)—, —NH—C₁₋₁₀alkylene-, —NH—C₁₋₁₀ alkylene-NH—C(O)—C₁₋₁₀ alkylene-, —NH—C₁₋₁₀alkylene-C(O)—NH—C₁₋₁₀alkylene-, —NH—(CH₂CH₂O)ᵤ—, —NH—(CH₂CH₂O)ᵤ—CH₂—, —NH—(CH₂CH₂NH)ᵤ—(CH₂)ᵤ—NH—(CH₂CH₂NH)ᵤ—(CH₂)ᵤ—NH—C(O)—(CH₂)ᵤ—NH—(C₃-C₈ carbocyclo)-, —NH-(arylene)-, and —NH—(C₃-C₈ heterocyclo-)-, wherein each u is independently 1-10.

In some embodiments, the Cleavable unit (—W_{w′}—) is present and may link the conjugator assembly to the Spacer unit when the Spacer unit is present or link the conjugator assembly to the Drug unit when the Spacer unit is absent. The linkage from the conjugator assembly to the Spacer unit or to the Drug unit can be directly from the conjugator assembly when the Stretcher unit is absent or via the Stretcher unit if the Stretcher unit is present.

In some embodiments, the Cleavable unit is directly conjugated to the conjugator assembly on one end and to the Drug unit on the other end. In some embodiments, the Cleavable unit is directly conjugated to the Stretcher unit on one end and to the Drug unit on the other end. In further embodiments, the Cleavable unit is directly conjugated to the Stretcher unit on one end and to the Spacer unit on the other end. In still further embodiments, the Cleavable unit is directly conjugated to the conjugator assembly on one end and to the Spacer unit on the other end. In embodiments, the Stretcher Unit and/or Spacer unit may be absent.

The Cleavable unit may form a cleavable bond with a Drug unit (PA) or a Spacer unit. Reactive groups for forming cleavable bonds can include, for example, sulfhydryl groups to form disulfide bonds; aldehyde, ketone, or hydrazine groups to form hydrazone bonds; carboxylic or amino groups to form peptide bonds; and carboxylic or hydroxy groups to form ester bonds.

The Cleavable unit may include a disulfide-containing linker cleavable through disulfide exchange, an acid-labile linker at acidic pH, or a linker cleavable by an enzyme such as a hydrolase, peptidase, esterase, or glucoronidase. The Cleavable unit may include one or more cleavage sites.

In some embodiments, the Cleavable unit includes one or more amino acids, such as 1 to 12. The Cleavable unit can include, for example, a monopeptide, dipeptide, tripeptide, tetrapeptide, pentapeptide, hexapeptide, heptapeptide, octapeptide, nonapeptide, decapeptide, undecapeptide, or dodecapeptide unit.

Each amino acid may be a natural or unnatural amino acid, and/or a D- or L-isomer of the same, provided that a cleavable bond is available. In some embodiments, the Cleavable unit includes only natural amino acids. Each amino acid may be a proteinogenic or non-proteinogenic amino acid.

In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, β-alanine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, and selenocysteine. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, and valine.

In some embodiments, each amino acid is independently selected from an L isomer of the following natural amino acids: alanine, arginine, aspartic acid, asparagine, histidine, glycine, glutamic acid, glutamine, phenylalanine, lysine, leucine, serine, tyrosine, threonine, isoleucine, tryptophan and valine. In some embodiments, each amino acid is a D isomer of the following natural amino acids: alanine, arginine, aspartic acid, asparagine, histidine, glycine, glutamic acid, glutamine, phenylalanine, lysine, leucine, serine, tyrosine, threonine, isoleucine, tryptophan and valine.

In embodiments, the Cleavable unit is the dipeptide -Val-Cit-, -Phe-Lys-, or -Val-Ala.

In some embodiments, the Cleavable unit includes one or two terminal amino acids and is linked to the Drug unit and/or Spacer unit through functional units present in a terminal amino acid, e.g., its carboxylic acid or amino termini.

In some embodiments, the bond between the Cleavable unit and the Drug unit can be enzymatically cleaved by one or more enzymes, including a tumor-associated protease, to liberate the Drug unit (-PA), which may be protonated in vivo upon release to provide a Drug (PA).

Useful Cleavable units may be designed to optimize their selectivity for enzymatic cleavage by a particular enzyme, such as a tumor-associated protease. In one embodiment, a linkage between the Cleavable unit and the Drug unit or Spacer unit is one for which cleavage is catalyzed by cathepsin B, C, and/or D, or a plasmin protease.

In some embodiments, the Spacer unit (—Y_{y′}—) is present and extends the framework of the covalent linker. A Spacer unit may link a Cleavable unit to the Drug unit or a Stretcher unit to the Drug unit or a conjugator assembly to a Drug unit. The Spacer unit may include one or more self-immolative or non-self-immolative groups. In some embodiments, the Spacer unit includes one or more self-immolative groups. In this context, the term “self-immolative group” refers to a bifunctional chemical moiety that is capable of covalently linking together two spaced chemical moieties into a normally stable tripartite molecule. The self-immolative group will spontaneously separate from the second chemical moiety if its bond to the first moiety is cleaved. In other embodiments, the Spacer unit is not self-immolative. In such embodiments, part or all of the Spacer unit remains attached to the Drug unit.

In some embodiments, —Y_{y′}— is a self-immolative group and is linked to a Cleavable unit via a methylene carbon atom of the self-immolative group, and linked directly to the Drug unit via a carbonate, carbamate, or ether group.

In some embodiments, —Y_{y′}— is a p-aminobenzyl alcohol (PAB) unit (e.g., —NH—(C₆H₄)—CH₂—O—C(═O)—). The phenylene portion of the PAB unit is optionally substituted with —C₁-C₈ alkyl, —O—(C₁-C₈ alkyl), -halogen, -nitro, or -cyano.

In some embodiments, —Y_{y′}— is —NH—C₁₋₁₀alkylene-O— or —NH-(p-C₆H₄)—C₁₋₁₀alkylene-O—.

In another embodiment, —Y_{y′}— is a carbonate group.

Other examples of self-immolative groups include, but are not limited to, aromatic compounds that are electronically similar to the PAB unit such as 2-aminoimidazol-5-methanol derivatives (see, e.g., Hay et al., 1999, Bioorg. Med. Chem. Lett. 9:2237) and ortho- or para-aminobenzylacetals. Suitable Spacer units include those that undergo cyclization upon amide bond hydrolysis, such as substituted and unsubstituted 4-aminobutyric acid amides (see, e.g., Rodrigues et al., 1995, Chemistry Biology 2:223), appropriately substituted bicyclo[2.2.1] and bicyclo[2.2.2] ring systems (see, e.g., Storm et al., 1972, J. Amer. Chem. Soc. 94:5815), and 2-aminophenylpropionic acid amides (see, e.g., Amsberry et al., 1990, J. Org. Chem. 55:5867). Elimination of amine-containing drugs that are substituted at the α-position of glycine (see, e.g., Kingsbury et al., 1984, J Med. Chem. 27:1447) are also examples of suitable self-immolative groups.

Other suitable Spacer units are disclosed in U.S. Publication No. 2005-0238649, the disclosure of which is incorporated by reference herein.

Suitable Stretcher units, Cleavable units, and Spacer units for use with the presently disclosed linkers, platforms, and ADCs care described in WO 2004/010957, WO 2007/038658, WO 2005/112919, U.S. Pat. Nos. 6,214,345, 7,659,241, 7,498,298, 7,968,687, and 8,163,888, and U.S. Publication Nos. 2009-0111756, 2009-0018086, and 2009-0274713, each of which is incorporated herein by reference in its entirety and for all purposes.

### Binding Agents

Provided herein are binding agents (BA or Ab), e.g., for use in an ADC described herein.

Compounds of Formula (I) may include any BA described herein.

In some embodiments, BA is an antibody or antigen binding fragment thereof, e.g., a humanized, chimeric, or human antibody or an antigen binding fragment thereof.

In some embodiments, the antibody or antigen binding fragment thereof specifically binds human B7H3. In some embodiments, the antibody or antigen binding fragment thereof is ifinatamab.

In some embodiments, the antibody or antigen binding fragment thereof specifically binds HER2. In some embodiments, the antibody or antigen binding fragment thereof is trastuzumab.

In some embodiments, the antibody or antigen binding fragment thereof specifically binds CLL1. In some embodiments, the antibody or antigen binding fragment thereof is 6E7.

In some embodiments, the antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)₂ fragment.

### Payloads

Provided herein are payloads (PA), e.g., for use in a platform and/or ADC described herein.

Compounds of Formula (I) may include any PA described herein

In some embodiments, each PA is independently a cytotoxic agent.

In some embodiments, each PA is independently selected from the group consisting of DXd, 7-ethyl-10-hydroxy-camptothecin (SN-38), and monomethyl auristatin E (MMAE).

In some embodiments, each PA independently is a compound of formula (VI):

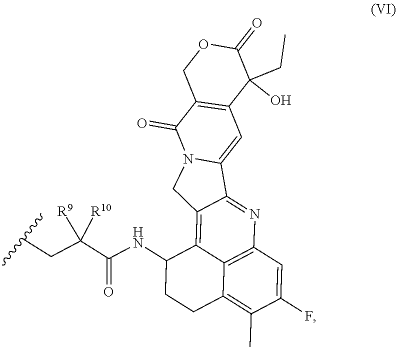

and each of R⁹ and R¹⁰ is independently hydrogen, halogen, or substituted or unsubstituted C₁₋₄ alkyl.

In some embodiments, each PA independently is

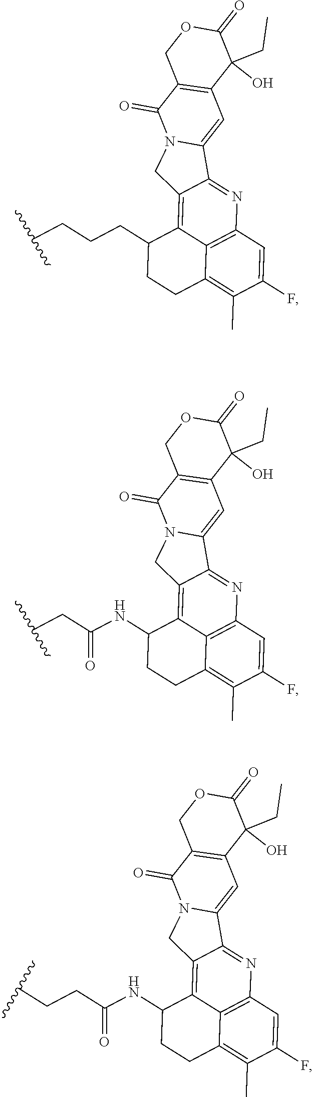 [SMILES: *CCCC1CCc2c(C)c(F)cc3nc4c(c1c23)Cn1c-4cc2c(c1=O)COC(=O)C2(O)CC ; *CC(=O)NC1CCc2c(C)c(F)cc3nc4c(c1c23)Cn1c-4cc2c(c1=O)COC(=O)C2(O)CC ; *CCC(=O)NC1CCc2c(C)c(F)cc3nc4c(c1c23)Cn1c-4cc2c(c1=O)COC(=O)C2(O)CC]

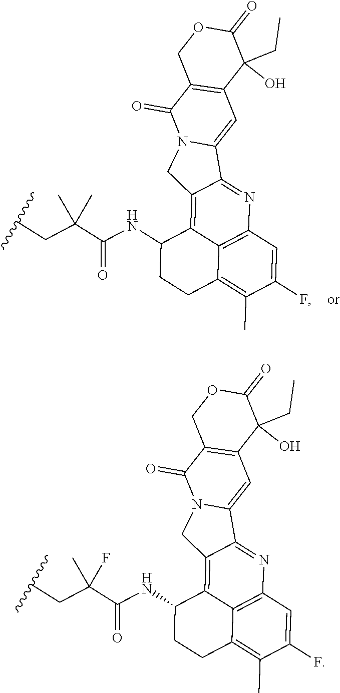

### 7.2.2. Aspect 2

In some embodiments, the ADC compound is represented by one of the following formulas, or a pharmaceutically acceptable salt, solvate, and/or stereoisomer.

| No. | Structure |
|---|---|
| | 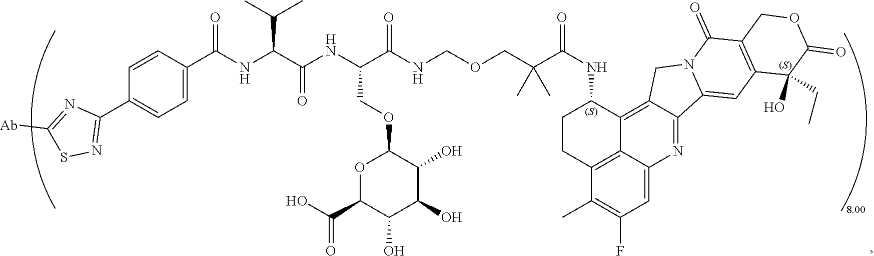 |
| | 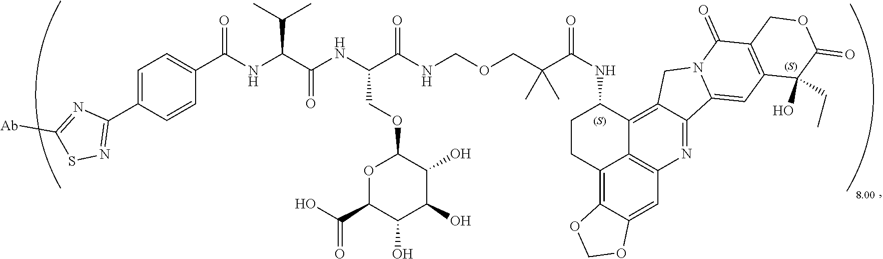 |
| | 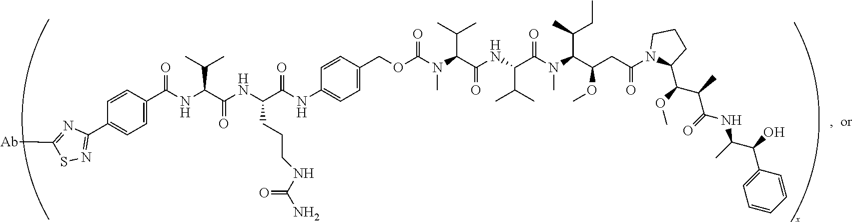 |
| | 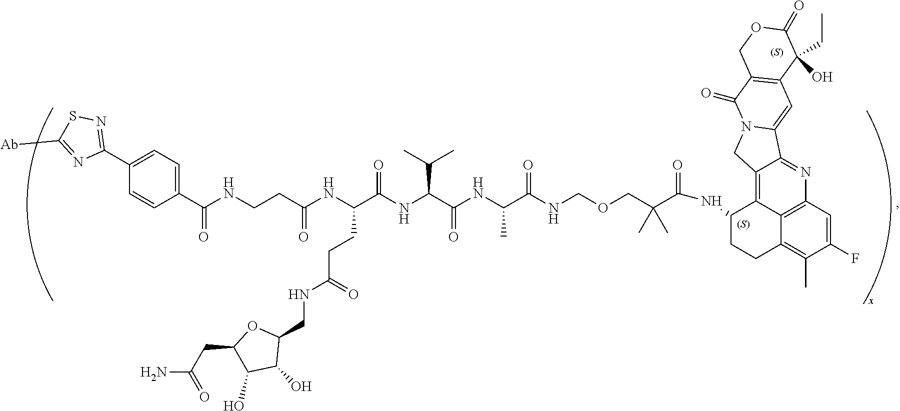 |

wherein Ab is a humanized, chimeric, or human antibody or an antigen binding fragment thereof and subscript x is from 1 to 15. Alternative values for Ab are as set forth herein, e.g., with respect to Aspect 1. Alternative values for variable subscript x are as set forth herein, e.g., with respect to compounds of Formula (I).

### 7.2.3. Aspect 3

Also provided herein are platforms, e.g., for use in preparing an ADC, such as an ADC described herein.

In some embodiments, the platform is a linker-payload compound of Formula (II):

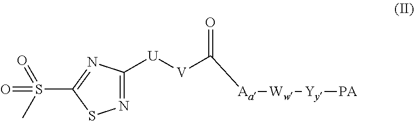

or a pharmaceutically acceptable salt, tautomer, solvate, or stereoisomer thereof, wherein:
U is arylene, heteroarylene, or a bond;
V is a bond or —C≡C—(CH₂)ₙ—;
n is an integer between 0 and 10;
A is a Stretcher unit residue;
subscript a′ is 0 or 1;
W is a Cleavable unit;
subscript w′ is 0 or 1;
Y is a Spacer unit;
subscript y′ is 0 or 1; and
PA is a payload residue.

In some embodiments, U and V are not each a bond at the same time.

In one embodiment, U is a bond. In one embodiment, U is arylene. In one embodiment, U is phenylene. In some embodiments, U is

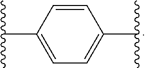 [SMILES: *c1ccc(*)cc1]

In one embodiment, U is heteroarylene. In one embodiment, U is a bivalent pyrimidine ring. In one embodiment, U is

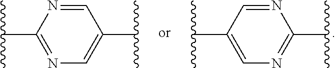 [SMILES: *c1cnc(*)nc1 ; *c1cnc(*)nc1]

In one embodiment, U is

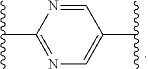 [SMILES: *c1cnc(*)nc1]

In one embodiment, U is

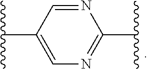 [SMILES: *c1cnc(*)nc1]

In one embodiment, V is a bond. In one embodiment, V is —C≡C—(CH₂)ₙ—. In one embodiment, V is —C≡C—(CH₂)₃—.

In some embodiments, A is —(CH₂)ₘ—C(═O)—, —CH₂—C(═O)—NH—(CH₂)ₘ—C(═O)—, —(CH₂CH₂O)ₘ—CH₂CH₂—C(═O)—, —CH[—(CH₂)ₘ—COOH]—C(═O)—, —CH₂—C(═O)—NH—(CH₂)ₘ—C(═O)—NH—(CH₂)ₘ—C(═O)—, —C(═O)—(CH₂)ₘ—C(═O)—, —NH—(CH₂)ₘ—C(═O)—, or —NH—(CH₂CH₂O)ₘ—CH₂CH₂—C(═O)—, wherein each m independently represents an integer of 1, 2, 3, 4, or 5.

In some embodiments, W is one of the following formulas:

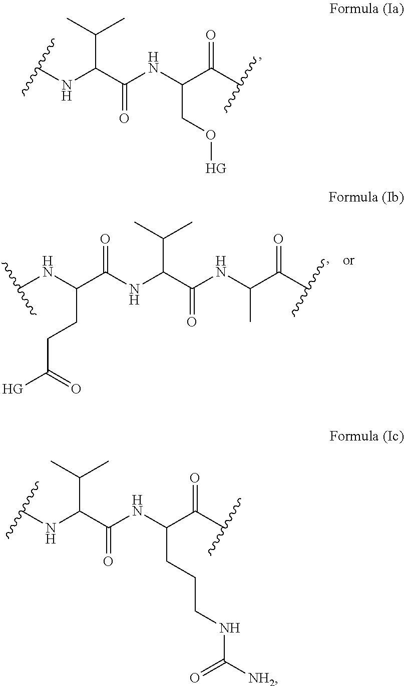

wherein HG is a hydrophilic moiety or hydrogen.

In some embodiments, HG is a saccharide, phosphate ester, sulfate ester, phosphodiester, or phosphonate.

In some embodiments, HG is a saccharide selected from β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid or any analogue or modification thereof, or sulfate, phosphate, carboxyl, amino, or O-acetyl modification thereof.

In some embodiments, HG is

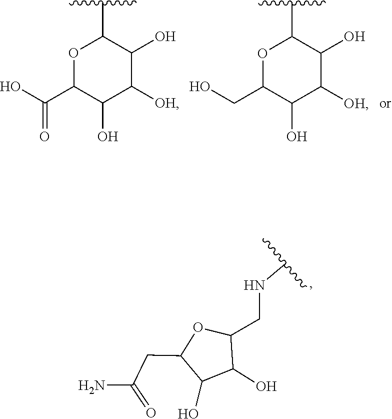 [SMILES: *C1OC(C(=O)O)C(O)C(O)C1O ; *C1OC(CO)C(O)C(O)C1O ; "or" ; *NCC1OC(CC(N)=O)C(O)C1O]

or a stereoisomer thereof. In some embodiments, HG is

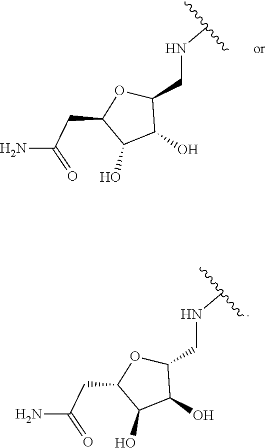 [SMILES: *NC[C@@H]1O[C@H](CC(N)=O)[C@@H](O)[C@H]1O ; *NC[C@H]1O[C@@H](CC(N)=O)[C@H](O)[C@@H]1O]

In some embodiments, Y is —NH—CH₂—O— or —NH-(p-C₆H₄)—CH₂—O—.

In some embodiments, the compound has one of the following formulas:

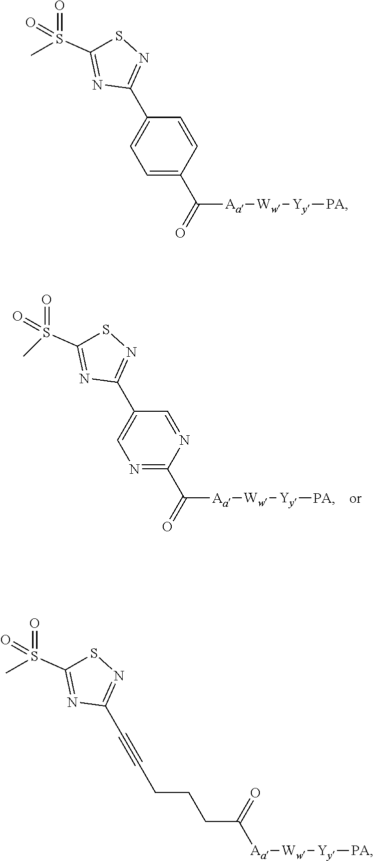

wherein values for the variables (e.g., PA, A, W, Y, a′, w′, y′, x) are as described above.

In some embodiments, the fraction

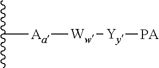

is one of the following formulas:

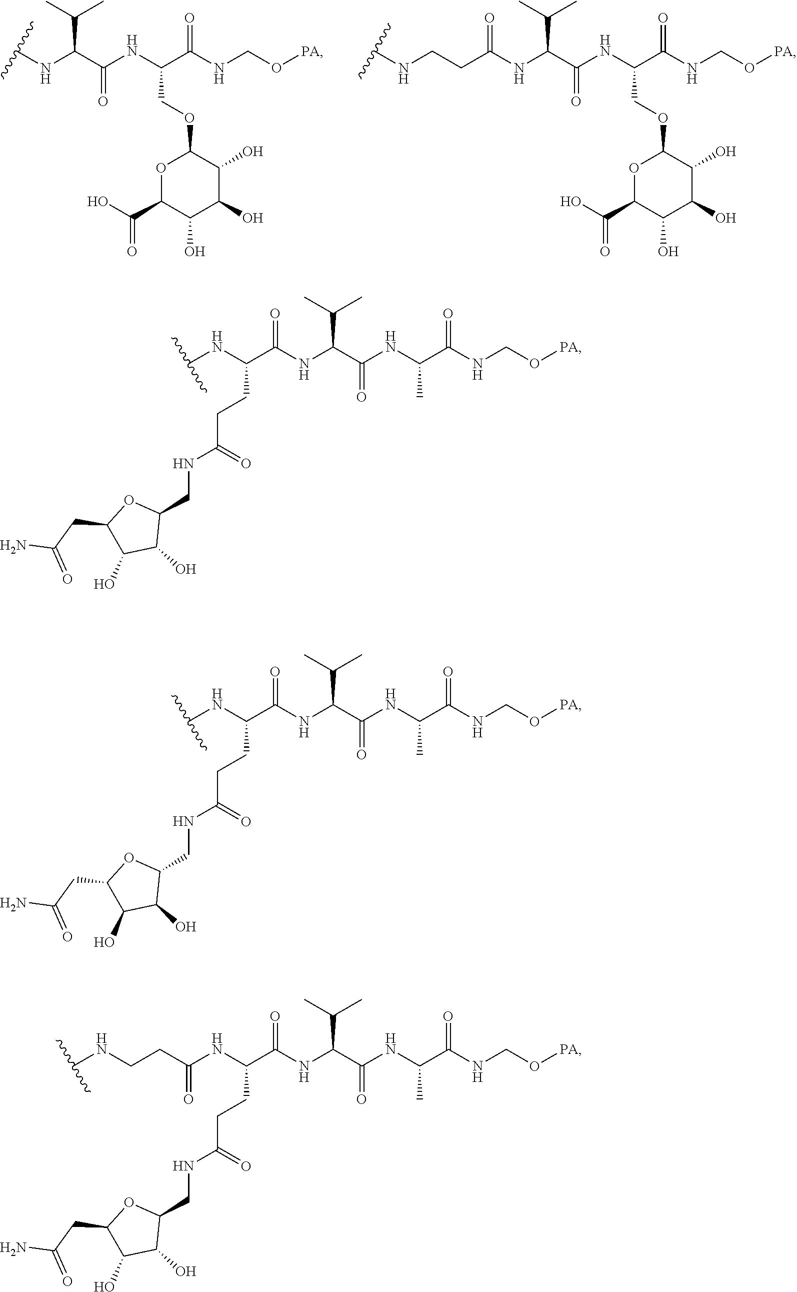

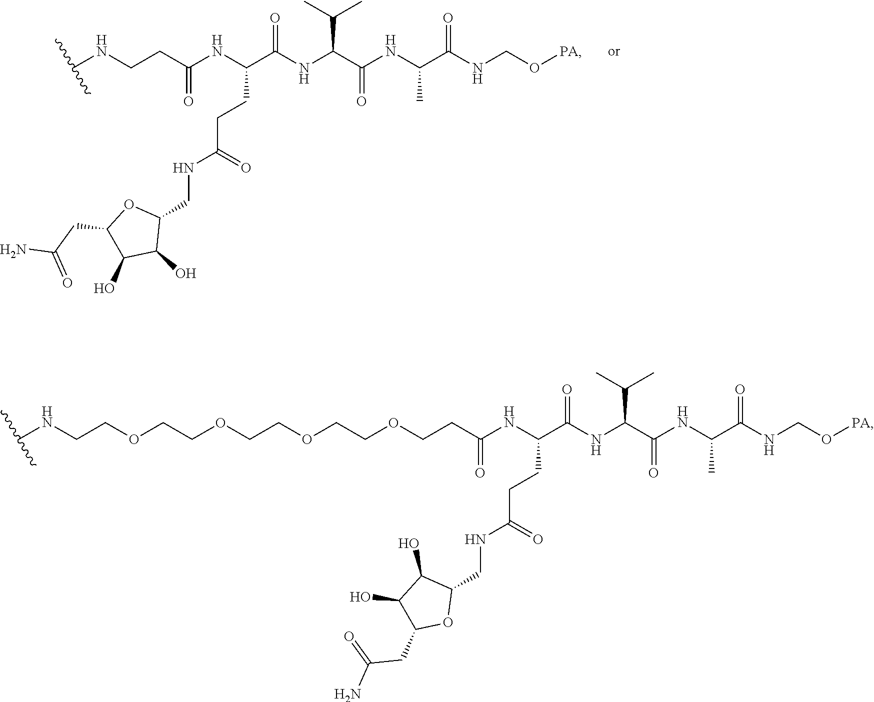

wherein PA is as described above.

In some embodiments, the compound is

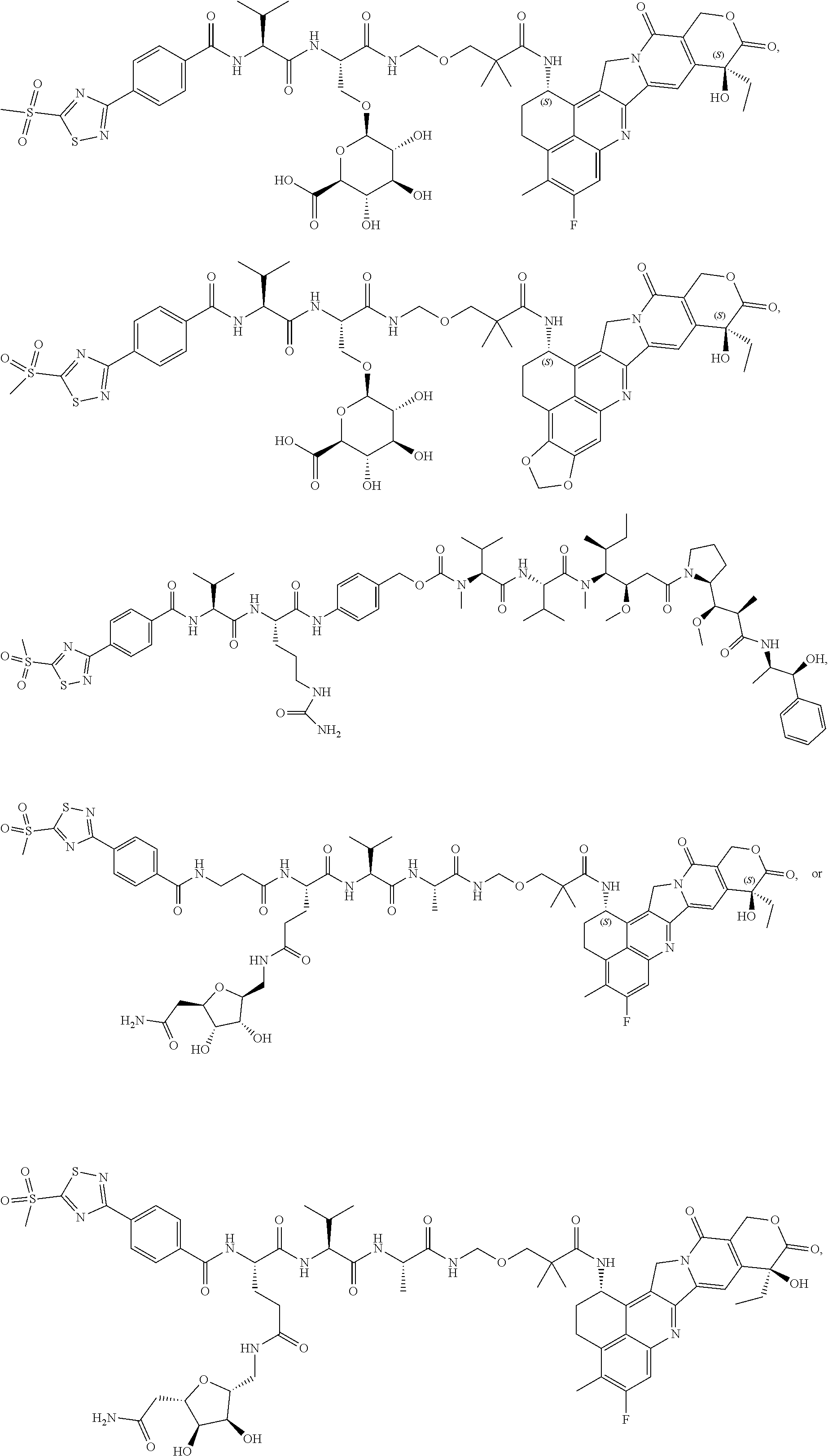

or a pharmaceutically acceptable salt, tautomer, or solvate thereof.

### 7.2.4. Aspect 4

Also provided herein are covalent linkers, e.g., for use in a platform and/or ADC described herein.

In some embodiments (e.g., of a linker for use in a platform), the linker is a compound of Formula (III):

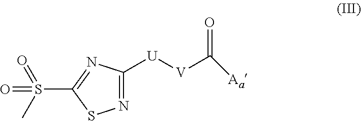

or a pharmaceutically acceptable salt, tautomer, solvate, or thereof, wherein:
U is arylene, heteroarylene, or a bond;
V is a bond or —C≡C—(CH₂)ₙ—;
n is an integer between 0 and 10;
A is a Stretcher unit; and
subscript a′ is 0 or 1.

In some embodiments, U and V are not each a bond at the same time.

In one embodiment, U is a bond. In one embodiment, U is arylene. In one embodiment, U is phenylene. In some embodiments, U is

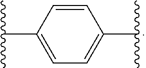 [SMILES: *c1ccc(*)cc1]

In one embodiment, U is heteroarylene. In one embodiment, U is a bivalent pyrimidine ring. In one embodiment, U is

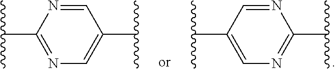 [SMILES: *c1cnc(*)nc1 ; *c1cnc(*)nc1]

In one embodiment, U is

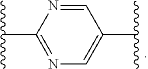 [SMILES: *c1cnc(*)nc1]

In one embodiment, U is

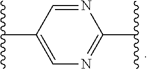 [SMILES: *c1cnc(*)nc1]

In one embodiment, V is a bond. In one embodiment, V is —C≡C—(CH₂)ₙ—. In one embodiment, V is —C≡C—(CH₂)₃—.

In some embodiments, A is a bond, —OH, —CH₃, —N(CH₃)₂, —(CH₂)ₘ—C(═O)R⁷, —CH₂—C(═O)—NH—(CH₂)ₘ—C(═O)R⁷, —(CH₂CH₂O)ₘ—CH₂CH₂—C(═O)R⁷, —CH[—(CH₂)ₘ—COOH]—C(═O)R⁷, —CH₂—C(═O)—NH—(CH₂)ₘ—C(═O)—NH—(CH₂)ₘ—C(═O)R⁷, —C(═O)—(CH₂)ₘ—C(═O)R⁷, —NH—(CH₂)ₘ—C(═O)R⁷, or —NH—(CH₂CH₂O)ₘ—CH₂CH₂—C(═O)R⁷, wherein each m independently represents an integer of 1, 2, 3, 4, or 5; R⁷ is OH or NR^{8a}R^{8b}; and each of R^{8a} and R^{8b} is, independently, H; substituted or unsubstituted C₁₋₄ alkyl; substituted or unsubstituted C₃₋₅ cycloalkyl; or R^{8a} and R^{8b} together with the atom to which they are attached form a substituted or unsubstituted C₃₋₅ cycloalkyl.

In some embodiments, R⁷ is OH, NH₂, NHCH₃, or N(CH₃)₂.

### 7.2.5. Aspect 5

In some embodiments, the linker compound is any one of the following, or a pharmaceutically acceptable salt and/or solvate thereof

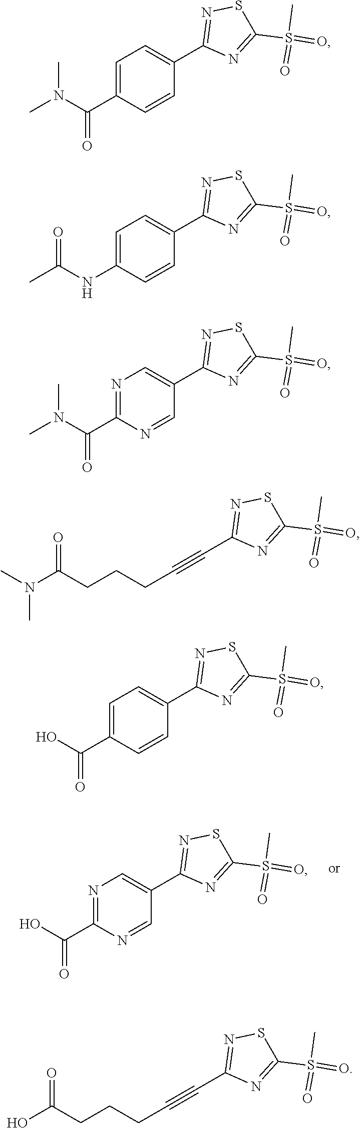

### 7.3. Methods or Processes of Making the Conjugates

Provided herein are methods of preparing a conjugate by contacting a binding agent (Ab) with a linker-payload compound under conditions suitable for forming a bond between the binding agent and the linker-payload compound. The reaction conditions may be any suitable reaction conditions known in the art. The binding agent may be an antibody and the bond may form an antibody-drug conjugate.

Examples of such reactions are provided in the Examples below.

In some embodiments, methods of making a conjugate including treating or contacting a compound with a binding agent under coupling conditions. The compound may include a reactive linker bonded to at least one payload. The compound may be any of the linker or platform compounds disclosed herein.

### 7.4. Pharmaceutical Compositions

Also provided herein are compositions, including pharmaceutical compositions, comprising an ADC set forth herein. In some embodiments, the compositions (e.g., pharmaceutical compositions) further comprise a pharmaceutically acceptable excipient.

Pharmaceutical compositions in accordance with the present disclosure can be prepared by mixing an antibody drug conjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Nos. U.S. Pat. No. 7,871,607 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody drug conjugate, which matrices are in the form of shaped articles, e.g. films, or microcapsules.

The formulations to be used for in vivo administration are generally sterile. Sterility can be readily accomplished, e.g., by filtration through sterile filtration membranes.

### 7.5. Methods of Using

In some embodiments, set forth herein is a method of treating a disease or disorder (e.g., a cancer) in a subject (e.g., patient) in need thereof, comprising administering to the patient an effective amount of an ADC disclosed herein.

The antibody drug conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injection, such as intravenous or subcutaneous injection, depending in part on whether the administration is brief or chronic. Various dosing schedules, including but not limited to, single or multiple administrations over various time-points, bolus administration, and pulse infusion are contemplated herein.

Antibody drug conjugates of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### 8. EXAMPLES

The examples below are intended to be exemplary and should not be considered limiting in any way. Unless otherwise specified, the experimental methods in the Examples described below are conventional methods. Unless otherwise specified, the reagents and materials are commercially available. All solvents and chemicals employed were of analytical grade or chemical purity. Solvents were redistilled before use. Anhydrous solvents were prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v) and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet (s), doublet (d), triplet (t), quartet (q), quintet (p), sextet (h), septet (hept), multiplet (m), or a combination thereof, br stands for broad. LC/MS data was recorded by using Agilent1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw®18.0.

For the sake of conciseness, certain abbreviations are used herein. One example is the single letter abbreviation to represent an amino acid. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

In the following examples, the following abbreviations are used:

| | |
|---|---|
| TEA | Triethyl amine |
| THF | Tetrahydrofuran |
| MeOH | Methanol |
| HOBt | 1-Hydroxybenzotriazole |
| DIPEA | N,N-Diisopropylethylamine |
| DMF | N,N-Dimethylformamide |
| LiOH | Lithium hydroxide |
| HCl | Hydrochloric acid |
| TFA | Trifluoroacetic acid |
| K₂CO₃ | Potassium carbonate |
| Na₂SO₄ | Sodium sulfate |
| EtOAc | Ethyl acetate |
| prep- | Preparative high performance liquid chromatography |
| HPLC | |
| Cu(OAc)₂ | Anhydrous cupric acetate |
| Pb(OAc)₄ | Lead tetraacetate |
| Et₂N | Diethylamine |
| Et₃N | triethylamine |
| r.t. | Room temperature |
| MS | Mass spectrometry |
| ESI | Electron spray ionization |
| FA | Formic acid |
| HPLC | High performance liquid chromatography |
| DMSO | Dimethylsulfoxide |
| DCM | Dichloromethane |
| EDCI | 1-Ethyl-3-(3′-dimethylaminopropyl)carbodiimide |
| PE | Polyethylene |
| EA | Ethyl acetate |
| m-CPBA | Meta-chloroperoxybenzoic acid |
| FCC | Flash column chromatography |
| Sc(OTf)₃ | Scandium(III) trifluoromethanesulfonate |
| Pd(dppf)Cl₂ | (1,1′-Bis(diphenylphosphine)ferrocene)palladium(II)di- |
| | chloride |

### UPLC Analysis Methods

Method A: Mobile phase A: 0.1% FA in water, B: MeCN; Gradient: 10% B maintain 0.2 min, 10%-95% B, 5.8 min, 95%₀B maintain 0.5 min; Flow rate: 0.6 mL/min; Column: ACQUITY UPLC® BEH C18 1.7 μm.

Method B: Mobile phase A: 0.100 FA in water, B: MeCN; Gradient: 100% B maintain 0.5 min, 10%-90% B, 2.5 min 90% B maintain 0.2 min; Flow rate: 0.6 mL/min; Column: ACQUITY UPLC® BEH C18 1.7 μm.

Method C: Mobile phase A: 0.1% FA in water, B: MeCN, Gradient: 100% B maintain 0.2 min, 10%-900% B, 1.3 min, 90% B maintain 0.3 min; Flow rate: 0.6 mL/min; Column: ACQUITY UPLC® BEH C18 1.7 μm.

### Example 1-1

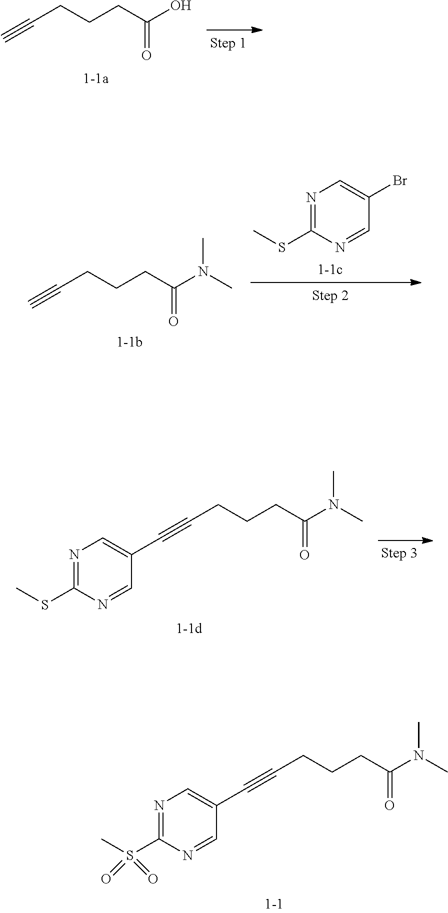

### Step 1: N,N-dimethylhex-5-ynamide (1-1b)

To a solution of compound 1-1a (500 mg, 4.46 mmol) in DCM (10 mL) were added dimethylamine hydrochloride (545 mg, 6.69 mmol), EDCI (1110 mg, 5.8 mmol), HOBt (783 mg, 5.8 mmol) and Et₃N (1350 mg, 13.4 mmol). The mixture was stirred at r.t. for 16 h. The mixture was diluted with EA (200 mL), washed with 1 N HCl (50 mL*3), sat. NaHCO₃ (50 mL*3) and brine (50 mL*3). The organic layer was dried over anhydrous Na₂SO₄, filtered, and concentrated to give the crude product 1-1b (458 mg, crude) as a light-yellow solid. MS (ESI) m/z: 140.2 [M+H]⁺.

### Step 2: N,N-dimethyl-6-(2-(methylthio)pyrimidin-5-yl)hex-5-ynamide (1-1d)

To a solution of compound 1-1b (100 mg, 0.72 mmol) in DMF (4 mL) were added compound 1-1c (162 mg, 0.79 mmol), CuI (14 mg, 0.072 mmol), Pd(PPh₃)₂Cl₂ (36 mg, 0.072 mmol), and Et₃N (1 mL). The mixture was stirred at 90° C. for 6 h under N₂ atmosphere. The mixture was filtered through a pad of celite, diluted with EA (100 mL), washed with brine (50 mL*4). The organic layer was dried over anhydrous Na₂SO₄, filtered, and concentrated. The crude product was purified by flash column chromatography (eluted with PE:EA=0-40%). Compound 1-1d (115 mg, 60.8% yield) was obtained as a light-yellow solid. MS (ESI) m/z: 264.1 [M+H]⁺.

### Step 3: N,N-dimethyl-6-(2-(methylsulfonyl)pyrimidin-5-yl)hex-5-ynamide (1-1)

To a solution of compound 1-1d (100 mg, 0.38 mmol) in DCM (4 mL) was added m-CPBA (262 mg, 1.52 mmol). The mixture was stirred at r.t. for 30 min. The mixture was diluted with EA (150 mL), washed with sat. NaHCO₃ (100 mL) and brine (100 mL*3). The organic layer was dried over anhydrous Na₂SO₄, filtered, and concentrated, The residue was purified by prep-HPLC (Method: column: XBridge Prep C18 OBD 5 um 19*250 mm; Mobile phase: A-water (0.1% TFA): B-acetonitrile; Flow rate: 20 mL/min).

Compound 1-1 (45.8 mg, 99% purity) was obtained as a white solid. MS (ESI) m/z: 296.2 [M+H]⁺.

### Example 1-2

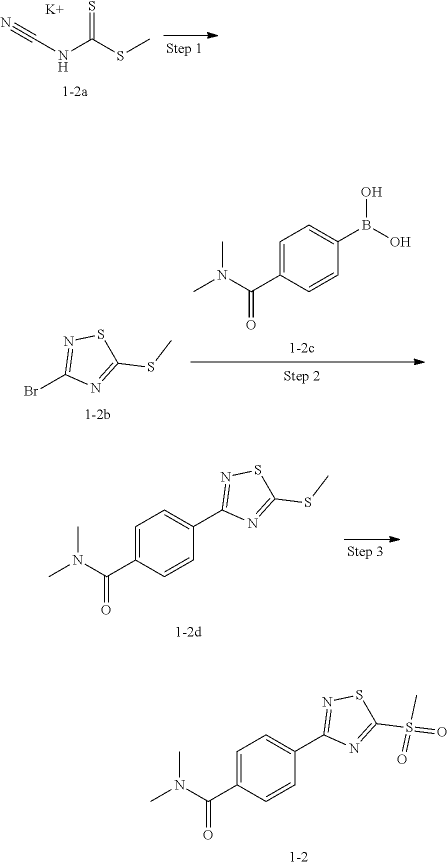

### Step 1: 3-bromo-5-(methylthio)-1,2,4-thiadiazole (1-2b)

To a solution of compound 1-2a (5 g, 29.4 mmol) in dichloromethane (100 mL) was added dropwise Br₂ (5.2 g, 32.9 mmol) at 0° C. After addition, the resulting mixture was stirred at room temperature overnight. To the reaction mixture was added excess Na₂SO₃ and water (50 mL) to decompose the excess Br₂. The mixture was separated, and the separated organic layer was washed with brine (50 mL×3), dried over Na₂SO₄ and concentrated in vacuo. The residue was purified by flash column chromatography (eluted with PE:EA=0%-40%) to give compound 1-2b (4.2 g, 68.2% yield) as a light-yellow solid. MS (ESI) m/z: 212.9 [M+H]⁺.

### Step 2: N,N-dimethyl-4-(5-(methylthio)-1,2,4-thiadiazol-3-yl)benzamide (1-2d)

To a solution of compound 1-2b (100 mg, 0.47 mmol) in DMF (4 mL) and H₂O (1 mL) were added compound 1-2c (110 mg, 0.57 mmol), K₃PO₄ (201 mg, 0.95 mmol), and Pd(PPh₃)₄ (36 mg, 0.072 mmol). The mixture was stirred at 90° C. for 6 h under N₂ atmosphere. The mixture was filtered through a pad of celite, diluted with EA (100 mL), washed with brine (50 mL*4). The organic layer was dried over anhydrous Na₂SO₄, filtered, and concentrated. The crude product was purified by flash column chromatography (eluted with PE:EA=0-40%). Compound 1-2d (35 mg, 26.4% yield) was obtained as white solid. MS (ESI) m/z: 280.2 [M+H]⁺.

### Step 3: N,N-dimethyl-4-(5-(methylsulfonyl)-1,2,4-thiadiazol-3-yl)benzamide (1-2)

To a solution of compound 1-2d (30 mg, 0.11 mmol) in DCM (4 mL) were added m-CPBA (74 mg, 0.43 mmol). The mixture was stirred at room temperature for 3 h. The mixture was concentrated and purified by prep-HPLC (Method: column: XBridge Prep C18 OBD Sum 19*250 mm; Mobile phase: A-water (0.1% TFA): B-acetonitrile; Flow rate: 20 mL/min). Compound 1-2 (4.0 mg, 12% yield) was obtained as a white solid.

MS (ESI) m/z: 312.3 [M+H]⁺.

### Example 1-3

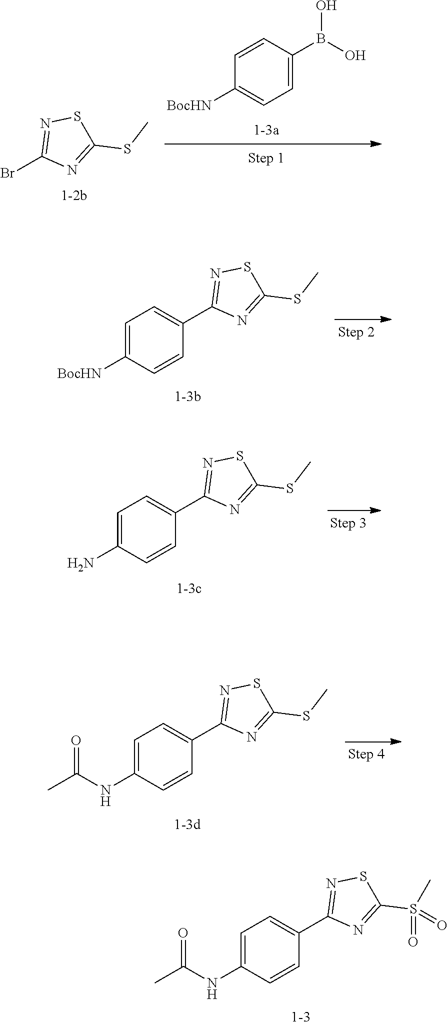

### Step 1: tert-butyl (4-(5-(methylthio)-1,2,4-thiadiazol-3-yl)phenyl)carbamate (1-3b)

To a solution of compound 1-2b (150 mg, 0.71 mmol) in toluene/EtOH (v:v=7:3, 5 mL) were added 1-3a (202 mg, 0.85 mmol), Na₂CO₃ (150 mg, 1.42 mmol) in H₂O (1 mL) and Pd(PPh₃)₄ (82 mg, 0.071 mmol). The mixture was heated to refluxing for 3 hr under N₂. After, the reaction mixture was cooled to room temperature and then diluted with 5.0 ml ethyl acetate. Then the aqueous layer was separated, the organic layer was washed with sat. NaCl solution, dried with Na₂SO₄, concentrated and then the residue was purified by flash column chromatography (eluted with PE:EA=0%-40%) to give compound 1-3b (75 mg, 32.6% yield).

MS (ESI) m/z: 324.1 [M+H]⁺.

### Step 2: 4-(5-(methylthio)-1,2,4-thiadiazol-3-yl)aniline (1-3c)

To a solution of compound 1-3b (75 mg, 0.23 mmol) in DCM (4 mL) was added TFA (1 mL) dropwise. The mixture was stirred at room temperature for 1 h. The mixture was concentrated and co-evaporated with toluene (3 mL*3). Compound 1-3b (55 mg, crude) was obtained as a white solid, which was used for the next step without further purification.

MS (ESI) m/z: 224.1 [M+H]⁺.

### Step 3: N-(4-(5-(methylthio)-1,2,4-thiadiazol-3-yl)phenyl)acetamide (1-3d)

To a solution of compound 1-3c (55 mg, 0.25 mmol) in THE (4 mL) was added acetic anhydride (50.3 mg, 0.49 mmol) and Et₃N (50 mg, 0.49 mmol). The mixture was stirred at room temperature for 2 h. The mixture was filtered, the filtrate was diluted with EA (50 mL), washed by sat. NaHCO₃ (50 mL*3), brine (50 mL*3), respectively. The organic layer was dried over anhydrous Na₂SO₄, filtered, and concentrated. The residue was purified by flash column chromatography (eluted with PE:EA=0%-40%). Compound 1-3d (50 mg, 76.5% yield) was obtained as a white solid.

MS (ESI) m/z: 266.1 [M+H]⁺.

### Step 4: N-(4-(5-(methylsulfonyl)-1,2,4-thiadiazol-3-yl)phenyl)acetamide (1-3)

To a solution of compound 1-3d (50 mg, 0.19 mmol) in DCM (4 mL) was added compound m-CPBA (130 mg, 0.75 mmol). The mixture was stirred at room temperature for 3 h. The mixture was concentrated and purified by prep-HPLC (Method: column: XBridge Prep C18 OBD 5 μm 19*250 mm; Mobile phase: A-water (0.1% TFA): B-acetonitrile; Flow rate: 20 mL/min). Compound 1-3 (12 mg, 12.7% yield) was obtained as a white solid.

MS (ESI) m/z: 298.1 [M+H]⁺.

### Example 1-4

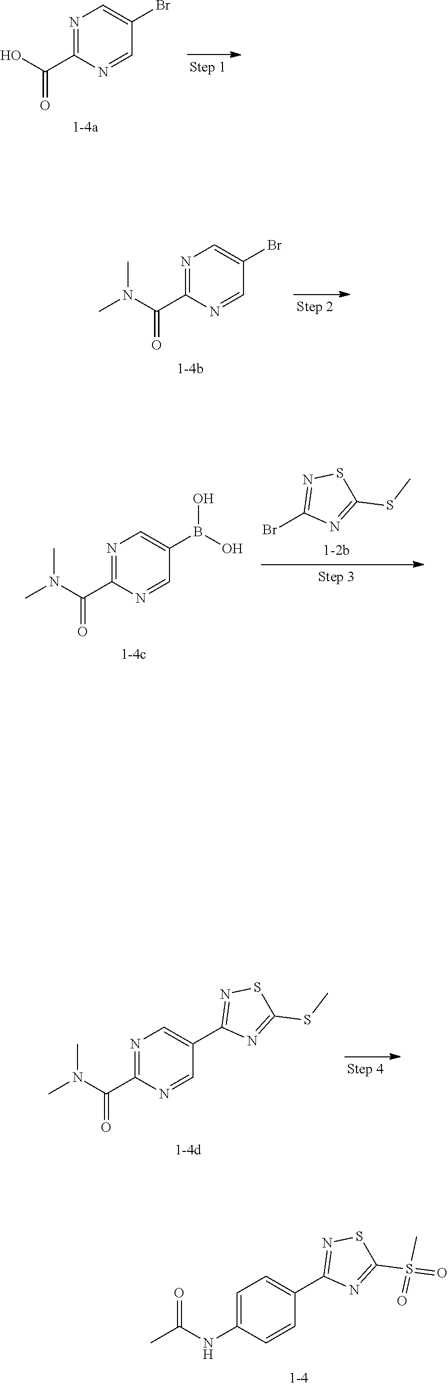

### Step 1: 5-bromo-N,N-dimethylpyrimidine-2-carboxamide (1-4b)

To a solution of compound 1-4a (2000 mg, 9.85 mmol) in DMF (40 mL) were added dimethylamine hydrochloride (1210 mg, 14.78 mmol), HATU (5620 mg, 14.78 mmol), and DIPEA (2550 mg, 19.70 mmol). The mixture was stirred at rt for 3 h under N₂ atmosphere. The mixture was concentrated. The crude product was purified by flash column chromatography (eluted with DCM: MeOH=0-20%). Compound 1-4b (1500 mg, 66.1% yield) was obtained as an off-white solid.

MS (ESI) m/z: 232.0 [M+H]⁺.

### Step 2: (2-(dimethylcarbamoyl)pyrimidin-5-yl)boronic acid (1-4c)

To a solution of compound 1-4b (1.4 g, 6.09 mmol) in 1,4-dioxane (30 mL) and H₂O (3 mL) were added 4,4,4′,4′,5,5,5′,5′-octamethyl-2,2′-Bi-(1,3,2-dioxaborolane) (1.85 g, 7.3 mmol) and potassium acetate (1.19 g, 12.2 mmol) under nitrogen atmosphere. The reaction mixture was stirred for 5 minutes and Pd(dppf)Cl₂·DCM (495 mg, 0.60 mmol) was added to it. The reaction mixture was refluxed for 16 h. After total consumption of starting material, the reaction mixture was diluted with water and extracted with ethyl acetate (3×100 mL). The combined organic layer was dried over Na₂SO₄, filtered, and purified by flash column chromatography (eluted with CH₂Cl₂:MeOH=0%-20%) to give compound 1-4c (1.05 g, 88.3% yield) as white solid.

MS (ESI) m/z: 196.0 [M+H]⁺.

### Step 3: N,N-dimethyl-5-(5-(methylthio)-1,2,4-thiadiazol-3-yl)pyrimidine-2-carboxamide (1-4d)

To a solution of compound 1-2c (150 mg, 0.71 mmol) in toluene/EtOH (v:v=7:3, 5 mL) were added 1-2b (166 mg, 0.85 mmol), Na₂CO₃ (150 mg, 1.42 mmol) in H₂O (1 mL) and Pd(PPh₃)₄ (82 mg, 0.071 mmol). The mixture was heated to refluxing for 3 hr under N₂. After, the reaction mixture was cooled to room temperature and then diluted with 5.0 ml ethyl acetate. Then the aqueous layer was separated, the organic layer was washed with sat. NaCl solution, dried with Na₂SO₄, concentrated and then the residue was purified by flash column chromatography (eluted with PE:EA=0%-40%) to give compound 1-4d (45 mg, 22.5% yield).

MS (ESI) m/z: 282.0 [M+H]⁺.

### Step 4: N,N-dimethyl-5-(5-(methylsulfonyl)-1,2,4-thiadiazol-3-yl)pyrimidine-2-carboxamide (1-4)

To a solution of compound 1-4d (40 mg, 0.14 mmol) in DCM (3 mL) was added m-CPBA (123 mg, 0.71 mmol). The mixture was stirred at room temperature for 2 h.

The mixture was concentrated and purified by prep-HPLC (Method: column: XBridge Prep C18 OBD Sum 19*250 mm; Mobile phase: A-water (0.1% TFA): B-acetonitrile; Flow rate: 20 mL/min). Compound 1-4 (3.7 mg, 9.3 yield) was obtained as a white solid. MS (ESI) m/z: 314.1 [M+H]+.

### Example 1-5

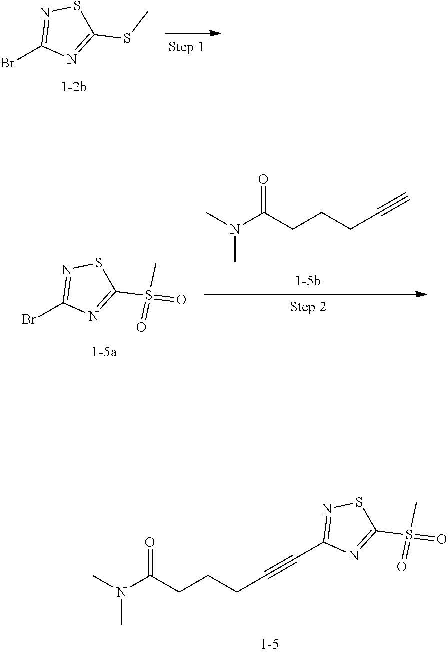

### Step 1: (3-bromo-5-(methylsulfonyl)-1,2,4-thiadiazole (1-5a)

To a solution of 1-2b (200 mg, 0.947 mmol) in DCM (3 mL) was added m-CPBA (817.5 mg, 4.737 mmol). The white suspension was stirred at r.t. for 5 hr. The mixture was quenched with sat.Na₂S₂O₃ (50 mL), extracted with DCM (30 mL*2). Combined organic layers were washed with brine (20 mL), dried over Na₂SO₄, filtered and the filtrate was concentrated under vacuum to give a residue. It was purified by FCC (EA/PE=0%˜20%), fraction was concentrated under vacuum to give 1-5a (239 mg, 95% yield) as a white solid.

¹H NMR (400 MHz, CDCl₃) δ 2.76 (s, 1H).

### Step 2: N,N-dimethyl-6-(5-(methylsulfonyl)-1,2,4-thiadiazol-3-yl)hex-5-ynamide (1-5)

To a mixture of 1-5a (239.0 mg, 0.983 mmol) and 1-5b (150.5 mg, 1.081 mmol) in DMF (4.0 mL) were added CuI (37.4 mg, 0.197 mmol), Pd(PPh₃)C₁₂ (69.0 mg, 0.098 mmol), and DIPEA (205 μL, 152.5 mg, 1.18 mmol) then degassed with nitrogen atmosphere three times. The mixture was stirred at r.t. overnight. The mixture was filtered and purified by prep-HPLC (0.1% FA), fraction was freeze-dried to give 1-5 (13.6 mg, 4% yield) as a yellow oil.

¹H NMR (400 MHz, CDCl₃) δ 3.38 (s, 3H), 3.01 (s, 3H), 2.95 (s, 3H), 2.60 (t, J=6.8 Hz, 2H), 2.50 (t, J=7.2 Hz, 1H), 2.00 (p, J=7.0 Hz, 2H).

MS (ESI) m/z: 302.0 [M+H]⁺.

¹H NMR (400 MHz, DMSO) δ 7.09 (s, 2H), 6.99 (t, J=6.4 Hz, 1H), 4.60 (dd, J=10.8, 4.0 Hz, 1H), 3.59-3.53 (m, 1H), 3.46-3.38 (m, 1H), 1.32 (s, 9H).

### Example 2-1

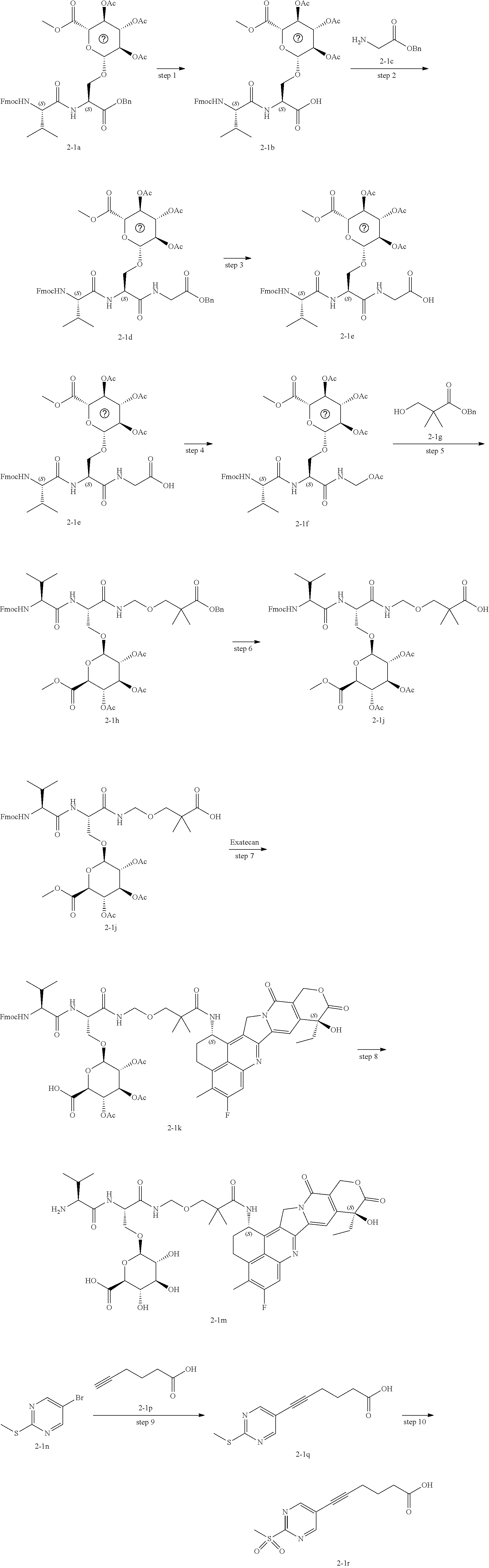

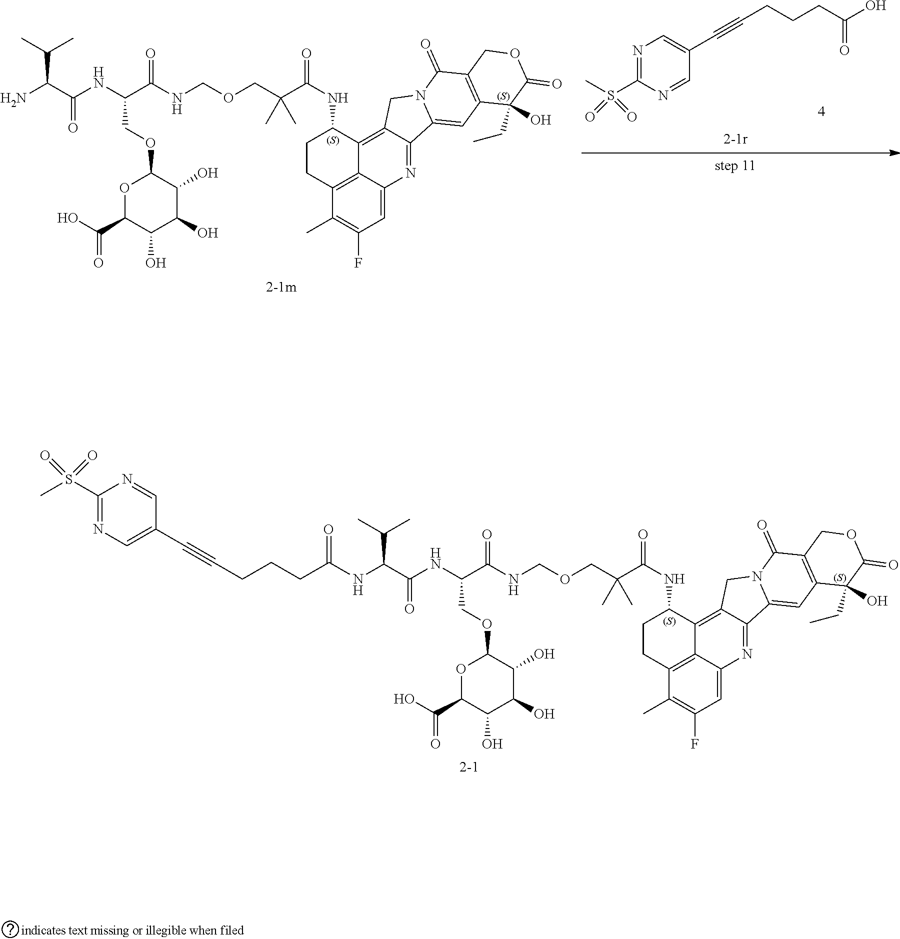

### Step 1: N-((((9H-fluoren-9-yl)methoxy)carbonyl)-L-valyl)-O—((2R,3R,4S,5S,6S)-3,4,5-triacetoxy-6-(methoxycarbonyl)tetrahydro-2H-pyran-2-yl)-L-serine (2-1b)

To a mixture of 2-1a (4.1 g, 4.92 mmol, purchased from MCE) in MeOH (50 mL), THE (100 mL) and DCM (20 mL) was added wet Pd/C (400 mg, 10% purity). The black suspension was purged with H₂ balloon three times and then stirred at r.t. for 1 hr. The black suspension was filtered through a pad of celite, washed with MeOH (200 mL). Organic layers were combined and concentrated under vacuum to give 2-1b (3650 mg, 99.8% yield) as an off-white solid.

MS (ESI) m/z: 743.6 [M+H]⁺.

### Step 2: (2R,3R,4S,5S,6S)-2-((S)-2-((S)-2-((((9H-fluoren-9-yl)methoxy)carbonyl)amino)-3-methylbutanamido)-3-((2-(benzyloxy)-2-oxoethyl)amino)-3-oxopropoxy)-6-(methoxycarbonyl)tetrahydro-2H-pyran-3,4,5-triyl triacetate (2-1d)

To a solution of 2-1b (3.65 g, 4.92 mmol) and 2-1c (1.66 g, 4.92 mmol) in DMF (50 mL) were added HATU (1.87 g, 4.92 mmol) and DIPEA (1.59 g, 12.29 mmol). The mixture was stirred at r.t. for 30 min. The mixture was purified by FCC (MeOH/DCM=0-10%), and the fraction was concentrated under vacuum to give 2-1d (3.8 g, 86.9% yield) as an off-white foamed solid.

MS (ESI) m/z: 890.7 [M+H]⁺.

### Step 3: N-((((9H-fluoren-9-yl)methoxy)carbonyl)-L-valyl)-O—((2R,3R,4S,5S,6S)-3,4,5-triacetoxy-6-(methoxycarbonyl)tetrahydro-2H-pyran-2-yl)-L-serylglycine (2-1e)

To a mixture of 2-1d (3.8 g, 4.27 mmol) in MeOH (150 mL) and DCM (50 mL) was added wet Pd/C (400 mg, 10% purity). The black suspension was purged with H₂ balloon three times and then stirred at r.t. for 40 min. The black suspension was filtered through a pad of celite, washed with MeOH (150 mL). Organic layers were combined and concentrated under vacuum to give 2-1e (3.3 g, 96.6% yield) as an off-white solid.

MS (ESI) m/z: 800.7 [M+H]⁺.

### Step 4: (2R,3R,4S,5S,6S)-2-((S)-2-((S)-2-((((9H-fluoren-9-yl)methoxy)carbonyl)amino)-3-methylbutanamido)-3-((acetoxymethyl)amino)-3-oxopropoxy)-6-(methoxycarbonyl)tetrahydro-2H-pyran-3,4,5-triyl triacetate (2-1f)

To a solution of 2-10e (3.3 g, 4.13 mmol) in DMF (30 mL) were added Pb(OAc)₄ (2.74 g, 6.19 mmol), Cu(OAc)2 (74.9 mg, 0.41 mmol), and HOAc (247.8 mg, 4.13 mmol). The resulting dark-colored mixture was purged with N₂ balloon three times and then stirred at 65° C. for 40 min, turning the mixture to deep blue. The mixture was diluted with EtOAc (300 mL), washed with brine (100 mL*3), dried over Na₂SO₄, filtered, and concentrated under vacuum to give a residue. It was purified by FCC (MeOH/DCM=0-10%), and the fraction was concentrated under vacuum to give 2-1f (2.8 g, 83.4% yield) as a pale-yellow solid.

MS (ESI) m/z: 836.6 [M+Na]⁺.

### Step 5: (2R,3R,4S,5S,6S)-2-((S)-2-((S)-2-((((9H-fluoren-9-yl)methoxy)carbonyl)amino)-3-methylbutanamido)-3-(((3-(benzyloxy)-2,2-dimethyl-3-oxopropoxy)methyl)amino)-3-oxopropoxy)-6-(methoxycarbonyl)tetrahydro-2H-pyran-3,4,5-triyl triacetate (2-1h)

A white suspension mixture of 2-1f (300 mg, 0.37 mmol), 2-1g (153.7 mg, 0.74 mmol) and 4 Å molecular sieve (200 mg) in anhydro THE (10 mL) was stirred at r.t. for 10 min. Sc(OTf)₃ (217.9 mg, 0.44 mmol) was added and the resulting yellow suspension was stirred at r.t. for 4 hr. The yellow suspension mixture was filtered through a pad of celite and washed with EA. Combined organic layers were washed with sat.NaHCO₃ (30 mL) and brine (30 mL), dried over Na₂SO₄, filtered, and the filtrate was concentrated under vacuum to give a residue. It was purified by silica gel column (MeOH/DCM=0%˜5%), and the fraction was concentrated under vacuum to give 2-1h (275 mg, 77.5% yield) as a white foam solid.

MS (ESI) m/z: 984.8 [M+Na]⁺.

### Step 6: (5S,8S)-1-(9H-fluoren-9-yl)-5-isopropyl-14,14-dimethyl-3,6,9-trioxo-8-((((2R,3R,4S,5S,6S)-3,4,5-triacetoxy-6-(methoxycarbonyl)tetrahydro-2H-pyran-2-yl)oxy)methyl)-2,12-dioxa-4,7,10-triazapentadecan-15-oic acid (2-1j)

To a solution of 2-1h (275 mg, 0.29 mmol) in MeOH (10 mL) was added wet Pd/C (55 mg, 10% purity). The black suspension was purged with H₂ balloon three times, then stirred at r.t. for 2 hr. The mixture was filtered through syringe head and washed with MeOH (15 mL), concentrated under vacuum to give 2-1j (230 mg, crude) as a white foam solid.

MS (ESI) m/z: 894.6 [M+Na]⁺.

### Step 7: (2R,3R,4S,5S,6S)-2-((S)-2-((S)-2-((((9H-fluoren-9-yl)methoxy)carbonyl)amino)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-6-(methoxycarbonyl)tetrahydro-2H-pyran-3,4,5-triyl triacetate (2-1k)

To a mixture of 2-10j (230 mg, crude), exatecan mesylate (139.9 mg, 0.26 mmol) and HATU (100.3 mg, 0.26 mmol) in DMF (5 mL) was added DIPEA (102.3 mg, 0.79 mmol). The resulting brown mixture was stirred at r.t. for 1 hr. The mixture was diluted with EtOAc (20 mL), washed with brine (20 mL*3), dried over Na₂SO₄, filtered, and concentrated under vacuum to give a residue. It was purified by FCC (MeOH/DCM=0%-3%), and concentrated under vacuum to give 2-1k (325 mg, 95.6% yield) as an off-white foam solid.

MS (ESI) m/z: 1289.9 [M+H]⁺.

### Step 8: (2S,3S,4S,5R,6R)-6-((S)-2-((S)-2-amino-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (2-1m)

To a solution of 2-1k (325 mg, 0.25 mmol) in DMF (5 mL) was added Et₂N (523.2 mg, 5.06 mmol). The mixture was stirred at r.t. for 20 min. After LCMS showed the reaction was completed, it was concentrated under vacuum to give a crude product. The crude product was dissolved in MeOH (6 mL). K₂CO₃ (174.7 mg, 1.26 mmol) was added and stirred at r.t. for 10 min, then H₂O (2 mL) was added to the mixture and stirred at r.t. for 30 min. The mixture was acidified with sat.KHSO₄ at 0° C. to pH=3, filtered, and purified by prep-HPLC (0.1% FA). The fraction was lyophilized to give 2-1m (140 mg, 59.7% yield) as a pale yellow solid.

MS (ESI) m/z: 927.4 [M+H]⁺.

¹H NMR (400 MHz, d₆-DMSO) δ 9.56 (s, 1H), 8.39 (s, 1H), 8.07 (d, J=8.4 Hz, 1H), 7.77 (d, J=11.2 Hz, 1H), 7.31 (s, 1H), 6.52 (s, 1H), 5.54 (dd, J=13.2, 7.2 Hz, 1H), 5.43 (s, 2H), 5.18 (dd, J=41.6, 18.8 Hz, 2H), 5.09-5.02 (m, 1H), 4.96 (s, 1H), 4.62 (dd, J=10.0, 6.8 Hz, 1H), 4.56-4.44 (m, 2H), 4.19 (d, J=7.6 Hz, 1H), 3.82 (dd, J=10.8, 6.8 Hz, 1H), 3.61 (dd, J=11.6, 6.4 Hz, 2H), 3.17-3.05 (m, 4H), 2.94 (t, J=8.0 Hz, 1H), 2.39 (s, 3H), 2.11 (dt, J=21.3, 7.6 Hz, 2H), 2.03-1.93 (m, 2H), 1.92-1.78 (m, 3H), 1.12 (d, J=8.0 Hz, 6H), 0.87 (dd, J=13.0, 6.6 Hz, 9H).

### Step 9: 6-(2-(methylthio)pyrimidin-5-yl)hex-5-ynoic acid (2-1q)

To a mixture of 2-1n (2.0 g, 9.751 mmol) and 2-1p (1.31 g, 11.702 mmol) in DMF (30 mL) were added CuI (185.7 mg, 0.975 mmol), Pd(PPh₃)C₁₂ (684.4 mg, 0.975 mmol) and TEA (4.1 mL, 2.960 g, 29.254 mmol). The mixture was degassed with nitrogen atmosphere three times, then stirred at 95° C. for 2 hr. The mixture was diluted with EtOAc (30 mL), washed with brine (30 mL*2), dried over Na₂SO₄, and the filtrate was concentrated under vacuum to give a residue. It was purified by FCC (MeOH/DCM=0%˜3%), and the fraction was concentrated under vacuum to give 2-1q (1.6 g, ˜65% yield) as a yellow solid.

MS (ESI) m/z: 237.1 [M+H]⁺.

### Step 10: 6-(2-(methylsulfonyl)pyrimidin-5-yl)hex-5-ynoic acid (2-1r)

To a solution of 2-1q (520 mg, 1.613 mmol) in DCM (10 mL) was added m-CPBA (1113.4 mg, 6.452 mmol). The resulting yellow suspension was stirred at r.t. for 1.5 hr. The mixture was diluted with EA (50 mL) and quenched by addition of sat.Na₂S₂O₃ (30 mL), stirred at r.t for 10 min, extracted with DCM (30 mL*3). Organic layers were combined and washed with brine (50 mL), dried over Na₂SO₄, filtered, and concentrated under vacuum to give crude product as a yellow solid. It was purified by prep-HPLC (FA), and the fraction was freeze-dried to give 2-1r (78.0 mg, 18% yield) as a white solid.

MS (ESI) m/z: 269.1 [M+H]⁺.

¹H NMR (400 MHz, d₆-DMSO) δ 12.17 (s, 1H), 9.12 (s, 2H), 3.41 (s, 3H), 2.60 (t, J=7.2 Hz, 2H), 2.41 (t, J=7.2 Hz, 2H), 1.82 (p, J=6.8 Hz, 2H).

### Step 11: (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-((S)-3-methyl-2-(6-(2-(methylsulfonyl)pyrimidin-5-yl)hex-5-ynamido)butanamido)-3-oxopropoxy)-3,4,5-trihydroxytetrahydro-2H-pyran-2-carboxylic acid (2-1)

To a solution of 2-1r (5.2 mg, 0.019 mmol) and 2-1m (15.0 mg, 0.016 mmol) in DMF (2 mL) were added HATU (8.6 mg, 0.024 mmol) and DIPEA (7 μL, 5.2 mg, 0.04 mmol). The mixture was stirred at r.t. for 30 min. The mixture was purified by prep-HPLC (FA 0.1%), and the fraction was freeze-dried to give 2-1 (7.1 mg, 37.3% yield) as a white solid.

MS (ESI) m/z: 1177.9 [M+H]⁺.

### Example 2-2

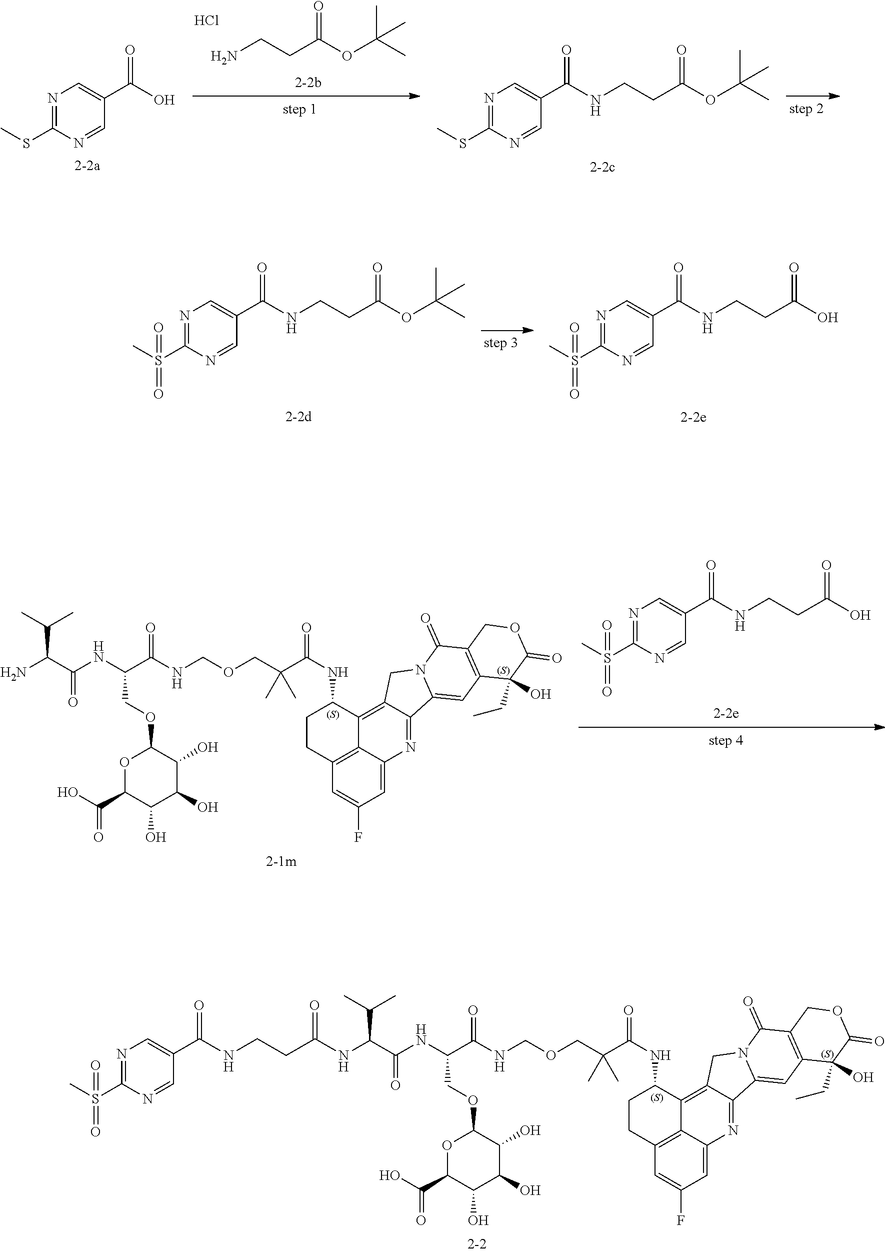

### Step 1: tert-butyl 3-(2-(methylthio)pyrimidine-5-carboxamido)propanoate (2-2c)

To a mixture of 2-2a (600 mg, 3.525 mmol) in DMF (15 mL) were added HATU (1621.9 mg, 4.266 mmol) and DIPEA (1.9 mL, 1503.5 mg, 11.633 mmol). The mixture was stirred at 45° C. for 10 min, 2-2b (704.6 mg, 3.878 mmol) was added and stirred at 45° C. for 40 min. The mixture was diluted with EtOAc (50 mL), washed with brine (30 mL*3), dried over Na₂SO₄, filtered and concentrated under vacuum to give a residue. It was purified by FCC (MeOH/DCM) to give 2-1c (1.3 g crude) as a brown oil.

MS (ESI) m/z: 298.4 [M+H]⁺.

### Step 2: tert-butyl 3-(2-(methylsulfonyl)pyrimidine-5-carboxamido)propanoate (2-2d)

To a solution of 2-2c (500 mg, crude) in DCM (10 mL) was added m-CPBA (1160.5 mg, 6.725 mmol). The white suspension was stirred at r.t. for 1 hr. The mixture was diluted with EA (50 mL) and quenched by addition of sat.Na₂S₂O₃ (30 mL), stirred at r.t for 10 min, extracted with DCM (30 mL*3). Organic layers were combined and washed with brine (50 mL), dried over Na₂SO₄, filtered, and concentrated under vacuum to give crude product as a yellow solid. It was purified by FCC (MeOH/DCM), and the fraction was concentrated under vacuum to give 2-2d (303 mg, -50% yield) as a white solid.

MS (ESI) m/z: 330.3 [M+H]⁺.

¹H NMR (400 MHz, cdcl₃) δ 9.27 (s, 2H), 7.38 (s, 1H), 3.74 (dd, J=11.6, 6.0 Hz, 1H), 3.40 (s, 2H), 2.60 (t, 1H), 1.47 (s, 5H).

### Step 3: 3-(2-(methylsulfonyl)pyrimidine-5-carboxamido)propanoic acid (2-2e)

A mixture of 2-2d (303.0 mg, 0.92 mmol) in TFA (1 mL) and DCM (2 mL) was stirred at r.t. for 1.5 hr. The mixture was concentrated under vacuum and co-evaporated with toluene three time to give 2-2e (255 mg) as an off-white solid. MS (ESI) m/z: 274.1 [M+H]⁺.

### Step 4: (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-((S)-3-methyl-2-(3-(2-(methylsulfonyl)pyrimidine-5-carboxamido)propanamido)butanamido)-3-oxopropoxy)-3,4,5-trihydroxytetrahydro-2H-pyran-2-carboxylic acid (2-2)

To a solution of 2-2e (7.1 mg, 0.026 mmol) and 2-1m (20.0 mg, 0.022 mmol) in DMF (2 mL) were added HATU (11.5 mg, 0.030 mmol) and DIPEA (9 μL, 7.0 mg, 0.054 mmol). The mixture was stirred at r.t. for 30 min. The mixture was purified by prep-HPLC (FA 0.1%), and the fraction was freeze-dried to give 2-2 (13.4 mg, ˜52.5% yield) as a white solid. MS (ESI) m/z: 1182.8 [M+H]⁺.

### Example 2-3

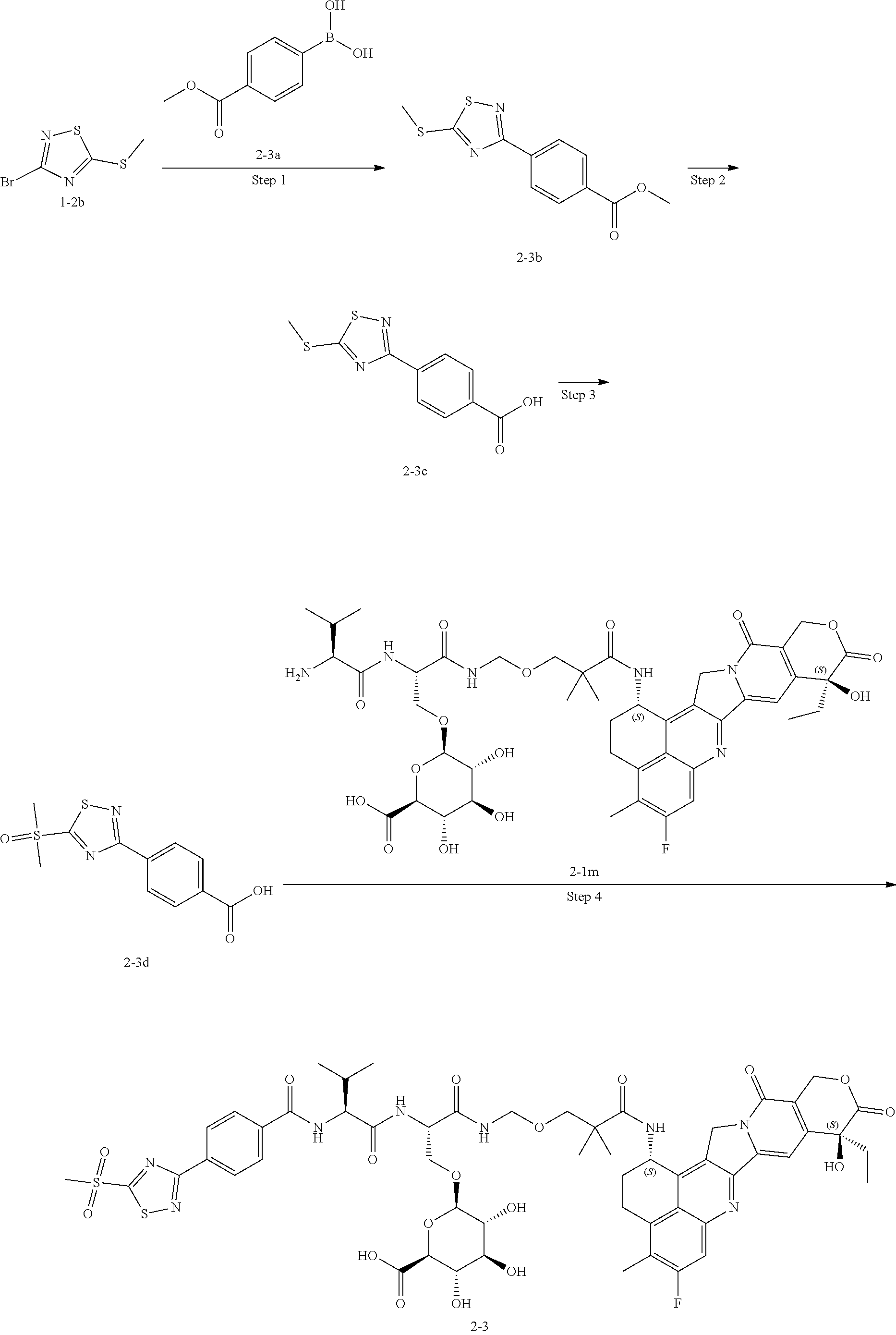

### Step 1: methyl 4-(5-(methylthio)-1,2,4-thiadiazol-3-yl)benzoate (2-3b)

To a solution of compound 1-2b (100 mg, 0.47 mmol) in toluene (4 mL) and H₂O (1 mL) were added compound 2-3a (109.72 mg, 0.568 mmol), K₂CO₃ (168 mg, 0.947 mmol) and Pd(dppf)C₁₂.DCM (34.6 mg, 0.047 mmol). The mixture was stirred at 110° C. for 3 h under N₂ atmosphere. The mixture was filtered through a pad of celite, diluted with EA (100 mL), washed with brine (50 mL*4). The organic layer was dried over anhydrous Na₂SO₄, filtered, and concentrated. The crude product was purified by flash column chromatography (eluted with PE:EA=0-40%). Compound 2-3b (56 mg, 44.4% yield) was obtained as an off-white solid.

MS (ESI) m/z: 267.1 [M+H]⁺.

### Step 2: 4-(5-(methylthio)-1,2,4-thiadiazol-3-yl)benzoic acid (2-3c)

To a solution of compound 2-3b (54 mg, 0.20 mmol) in MeOH (3 mL) and H₂O (1 mL) was added LiOH (17 mg, 0.41 mmol). The mixture was stirred at r.t. for 2 h. The mixture was adjusted to pH 7 and purified by prep-HPLC (FA condition) to give compound 2-3c (36 mg, 70.3% yield) as a white solid.

MS (ESI) m/z: 253.1 [M+H]⁺.

### Step 3: 4-(5-(methylsulfonyl)-1,2,4-thiadiazol-3-yl)benzoic acid (2-3d)

To a solution of compound 2-3c (35 mg, 0.14 mmol) in DCM (3 mL) and THE (3 mL) was added m-CPBA (96 mg, 0.55 mmol). The mixture was stirred at room temperature for 16 h. The mixture was concentrated and purified by prep-HPLC (Method: column: XBridge Prep C18 OBD Sum 19*250 mm; Mobile phase: A-water (0.1% TFA): B-acetonitrile; Flow rate: 20 mL/min). Compound 2-3d (12 mg, 99% purity) was obtained as a white solid.

MS (ESI) m/z: 284.8 [M+H]⁺.

### Step 4: (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-((S)-3-methyl-2-(4-(5-(methylsulfonyl)-1,2,4-thiadiazol-3-yl)benzamido)butanamido)-3-oxopropoxy)-3,4,5-trihydroxytetrahydro-2H-pyran-2-carboxylic acid (2-3)

To a solution of compound 2-3d (7.36 mg, 0.026 mmol) in DMF (2 mL) were added HATU (9.02 mg, 0.024 mmol) and DIPEA (5.58 mg, 0.043 mmol). The mixture was stirred at r.t. for 30 min. Compound 2-1m (20 mg, 0.022 mmol) was added to the mixture and stirred at r.t. for 15 min. The reaction was purified by prep-HPLC (Method: column XBridge Prep C18 OBD 5 μm 19*250 mm; Mobile phase: A-water (0.1% TFA): B-acetonitrile; Flow rate: 20 mL/min) to give compound 2-3 (7.6 mg, 29.5% yield) as a white solid.

MS (ESI) m/z: 1193.5 [M+H]⁺.

### Example 2-4

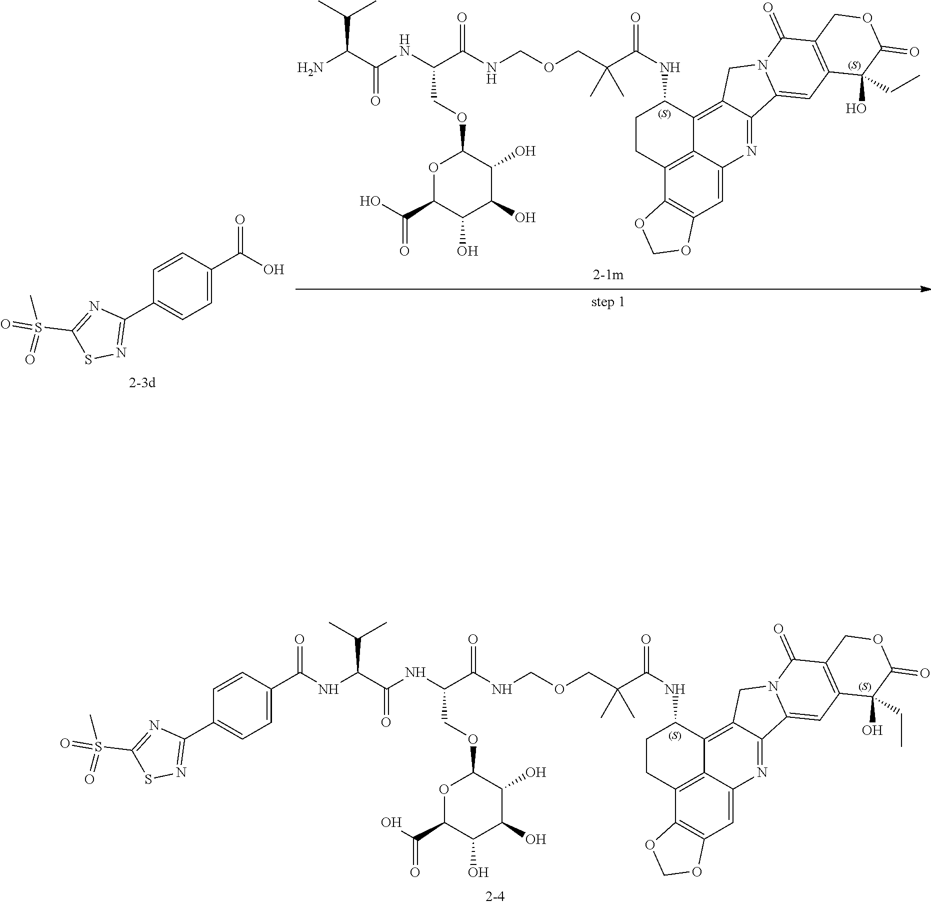

Step 1: (2S,3S,4S,5R,6R)-6-((S)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2,2-dimethyl-3-oxopropoxy)methyl)amino)-2-((S)-3-methyl-2-(4-(5-(methylsulfonyl)-1,2,4-thiadiazol-3-yl)benzamido)butanamido)-3-oxopropoxy)-3,4,5-trihydroxytetrahydro-2H-pyran-2-carboxylic acid (2-4)

To a solution of 2-3d (5.9 mg, 0.021 mmol) in dry DMF (0.5 mL) were added HATU (7.9 mg, 0.021 mmol) and DIPEA (0.008 mL, 0.043 mmol), stirred at r.t. for 15 min. Then to the above mixture was added 2-1m (trifluoroacetate) (18.0 mg, 0.017 mmol), stirred at r.t. for 10 min. The resulting solution was purified by prep-HPLC (Method: column XBridge Prep C18 OBD Sum 19*150 mm; Mobile phase: A-water (0.1% TFA): B-acetonitrile; Flow rate: 20 mL/min), and the fraction was lyophilized to give 3-1 (5.5 mg, 26.7% yield) as a yellow solid.

MS (ESI) m/z: 1205.6 [M+H]⁺.

### Example 2-5

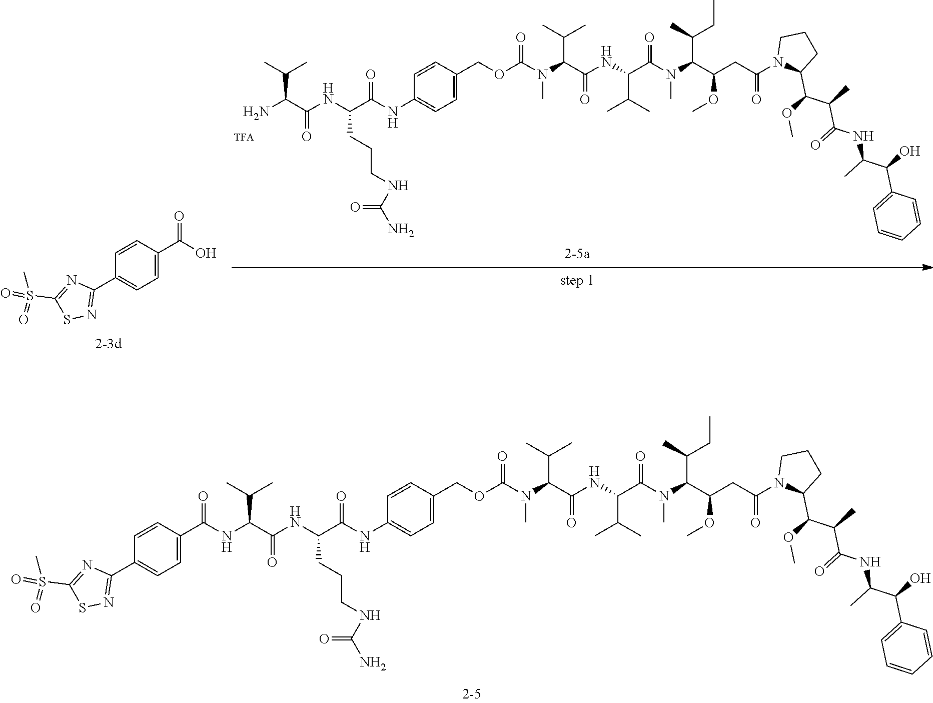 [SMILES: CS(=O)(=O)c1nc(-c2ccc(C(=O)O)cc2)ns1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2ccc(-c3nsc(S(C)(=O)=O)n3)cc2)C(C)C)cc1)C(C)C]

4-((S)-2-((S)-3-methyl-2-(4-(5-(methylsulfonyl)-1,2,4-thiadiazol-3-yl)benzamido)butanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (2-5)

To a solution of 2-3d (10 mg, 0.035 mmol) in dry DMF (1 mL) were added HATU (15 mg, 0.040 mmol) and DIPEA (9.1 mg, 0.070 mmol). The mixture was stirred at r.t. for 15 min. Then to the above mixture was added 2-5a (TFA salt, commercially available) (36 mg, 0.032 mmol) and further stirred at r.t. for 10 min. The resulting solution was purified by prep-HPLC (Method: column XBridge Prep C18 OBD Sum 19*150 mm; Mobile phase: A-water (0.1% TFA): B-acetonitrile; Flow rate: 20 mL/min), and the fraction was lyophilized to give 2-5 (14 mg, 31.8% yield) as a white solid.

MS (ESI) m/z: 1389.8 [M+H]⁺.

### Example 2-6

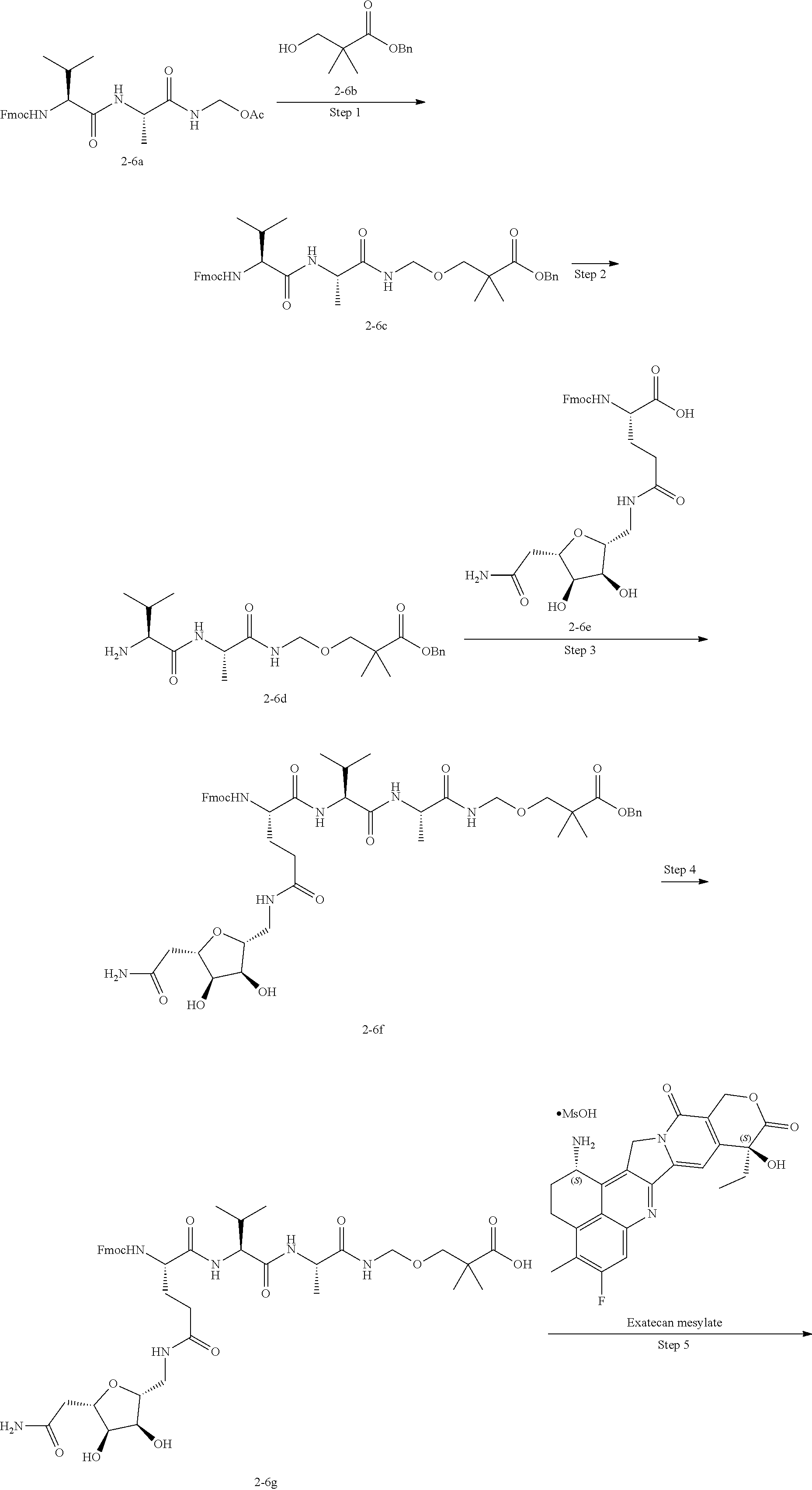

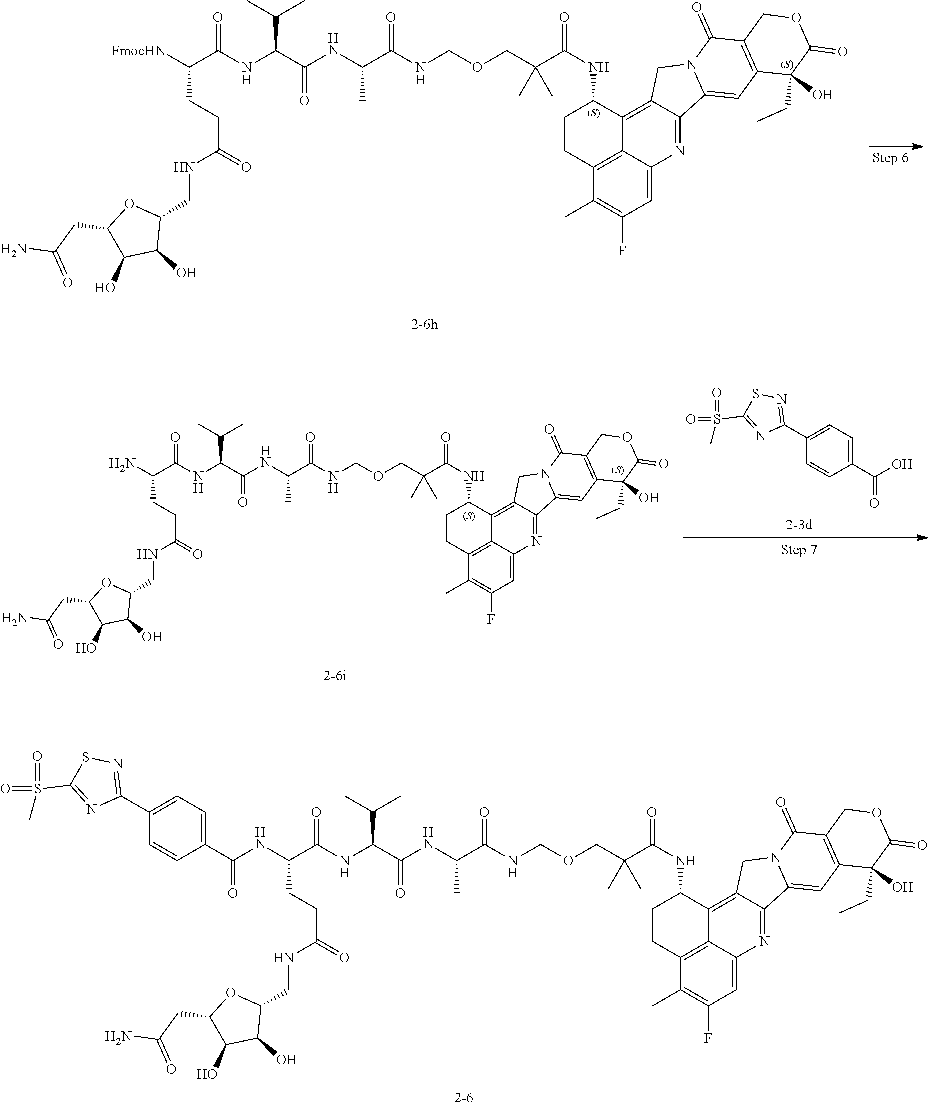

### Step 1: benzyl (5S,8S)-1-(9H-fluoren-9-yl)-5-isopropyl-8,14,14-trimethyl-3,6,9-trioxo-2,12-dioxa-4,7,10-triazapentadecan-15-oate (2-6c)

A white suspension mixture of 2-6a (300 mg, 0.623 mmol), 2-6b (259.6 mg, 1.246 mmol), and 4 Å molecular sieves in anhydro THE (10 mL) was stirred at r.t. for 10 min. Sc(OTf)₃ (368.0 mg, 0.748 mmol) was added and the resulting yellow suspension was stirred at r.t. for 4 hr. The yellow suspension mixture was filtered through a pad of celite and washed with EtOAc (30 mL). Combined organic layers were washed with sat.NaHCO₃ (30 mL) and brine (30 mL), dried over Na₂SO₄, filtered, and the filtrate was concentrated under vacuum to give a residue. It was purified by silica gel column (MeOH/DCM=0%˜5%), and the fraction was concentrated under vacuum to give 2-6c (274 mg, 69.8% yield) as a white solid.

MS (ESI) m/z: 652.6 [M+Na]⁺.

### Step 2: benzyl 3-(((S)-2-((S)-2-amino-3-methylbutanamido)propanamido)methoxy)-2,2-dimethylpropanoate (2-6d)

To a solution of 2-6c (274.0 mg, 0.44 mmol) in DMF (5 mL) was added Et₂N (477.3 mg, 5.53 mmol). The mixture was stirred at r.t. for 20 min. The reaction mixture was concentrated under vacuum and co-evaporated with toluene two times to give 2-6d (275.3 mg, crude) as a brown oil,

MS (ESI) m/z: 430.4 [M+Na]⁺.

### Step 3: benzyl (5S,8S,11S)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-8-isopropyl-11,17,17-trimethyl-3,6,9,12-tetraoxo-2,15-dioxa-4,7,10,13-tetraazaoctadecan-18-oate (2-6f)

To a solution of 2-6d (275.3 mg, crude) and 2-6e (282.3 mg, 0.52 mmol) in DMF (5 mL) were added HATU (198.2 mg, 0.52 mmol) and DIPEA (168.4 mg, 1.30 mmol). The mixture was stirred at r.t. for 10 min. The mixture was purified by reverse phase (C18, 60 g, 30%-70%), and the fraction was freeze-dried to give 2-6f (370 mg, 91.5% yield) as a brown solid.

MS (ESI) m/z: 953.8 [M+Na]⁺.

### Step 4: (5S,8S,11S,17R)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-17-fluoro-8-isopropyl-11,17-dimethyl-3,6,9,12-tetraoxo-2,15-dioxa-4,7,10,13-tetraazaoctadecan-18-oic acid (2-6g)

To a solution of compound 2-6f (3.0 g, 3.21 mmol) in co-solvent DMF-MeOH (40 mL, 1:1, v:v) was added Pd/C (10%, 600 mg). The mixture was stirred at H₂ atmosphere (15 psi) for 7 h. The mixture was filtered through a pad of celite, and concentrated to give compound 2-6g (2.5 g, crude) as a white solid.

MS (ESI) m/z: 863.7 [M+Na]⁺.

### Step 5: (9H-fluoren-9-yl)methyl ((6S,9S,12S)-1-((2R,3S,4R,5S)-5-(2-amino-2-oxoethyl)-3,4-dihydroxytetrahydrofuran-2-yl)-19-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-9-isopropyl-12,18,18-trimethyl-3,7,10,13,19-pentaoxo-16-oxa-2,8,11,14-tetraazanonadecan-6-yl)carbamate (2-6h)

To a solution of exatecan mesylate (1000 mg, 1.18 mmol) in DMF (20 mL) were added compound 2-6g (692 mg, 1.3 mmol), HATU (675 mg, 1.78 mmol), and DIPEA (459 mg, 3.55 mmol). The mixture was stirred at r.t. for 30 min. The mixture was concentrated and purified by a silica gel column chromatography (eluent: DCM/MeOH=100/0 to 20/80) to give the title compound 2-6h (1320 mg, 88.6% yield) as an off-white solid.

MS (ESI) m/z: 1282.1 [M+Na]⁺.

### Step 6: (S)-2-amino-N5-(((2R,3S,4R,5S)-5-(2-amino-2-oxoethyl)-3,4-dihydroxytetrahydrofuran-2-yl)methyl)-N1-((S)-1-(((S)-1-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-1-oxopropan-2-yl)amino)-3-methyl-1-oxobutan-2-yl)pentanediamide (2-6i)

To a solution of compound 2-6h (1000 mg, 0.793 mmol) in DMF (20 mL) was added Et₂NH (580 mg, 7.93 mmol). The mixture was stirred at r.t. for 30 min. The mixture was concentrated under high vacuum to give compound 2-6i (824.6 mg, crude) as an off-white solid, which was used directly without further purification.

MS (ESI) m/z: 1036.9 [M+H]⁺.

### Step 7: (S)—N₅-(((2R,3S,4R,5S)-5-(2-amino-2-oxoethyl)-3,4-dihydroxytetrahydrofuran-2-yl)methyl)-N1-((S)-1-(((S)-1-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-1-oxopropan-2-yl)amino)-3-methyl-1-oxobutan-2-yl)-2-(4-(5-(methylsulfonyl)-1,2,4-thiadiazol-3-yl)benzamido)pentanediamide (2-6)

To a solution of 2-3d (15.2 mg, 0.053 mmol) in dry DMF (1.0 mL) were added HATU (22.2 mg, 0.058 mmol) and DIPEA (0.017 mL, 0.097 mmol), stirred at r.t. for 15 min. Then to the above mixture was added 2-6i (50.0 mg, 0.048 mmol), stirred at r.t. for 10 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min), and the fraction was lyophilized to give 2-6 (32 mg, 50.9% yield) as a yellow solid.

MS (ESI) m/z: 1303.0 [M+H]⁺.

The compounds of Examples 1-1 to 1-5 are shown below in Table 1. The compounds of Examples 2-1 to 2-6 are shown below in Table 2.

### Conjugator-Antibody Conjugate and Antibody Drug Conjugate Preparation

DAR8 antibody drug conjugate/conjugator-antibody conjugate preparation. Antibody in conjugation buffer (with concentration 0.5-25 mg/mL, PBS buffer pH 6.0-8.5) was incubated under reduction temperature (0-40° C.) for 10 min. 8-15 eq. TECP solution (5 mM stock in PBS buffer) was added to the reaction mixture and the reduction reaction was left for 1-8 hours at reduction temperature. Organic solvent (e.g., DMSO, DMF, DMA, PG, acetonitrile, 0-25% v/v) and conjugator-linker-payload (see Table 2) or conjugator (see Table 1) stock (10-25 eq, 10 mM stock in organic solvent) were added stepwise after the reduction mixture was cooled to 0-25° C. Conjugation solution was left for 1-3 h at 0-25° C., and the reaction was quenched with N-acetyl cysteine (1 mM stock). The solution was submitted to buffer exchange (spin desalting column, ultrafiltration, and dialysis) into storage buffer (for example, pH 5.5-6.5 histidine acetate buffer, with optional additive such as sucrose, trehalose, tween 20, 60, 80).

Conjugator-antibody conjugates and ADCs prepared according to the foregoing method are shown in Tables 3 and 4, respectively.

ADC characterization. ADCs were characterized using the following analytical methods. Drug to antibody ratios (DAR) of the ADCs were determined by LCMS method or HIC method. SEC purity of ADCs made were all>95% purity.

LCMS method: LC-MS analysis was carried out under the following measurement conditions:

LC-MS system: Vanquish Flex UHPLC and Orbitrap Exploris 240 Mass Spectrometer

Column: MAbPac™ RP, 2.1*50 mm, 4 μm, 1,500 Å, Thermo Scientific™

Column temperature: 80° C.

Mobile phase A: 0.1% formic acid (FA) aqueous solution

Mobile phase B: Acetonitrile solution containing 0.1% formic acid (FA)

Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)

Injected sample amount: 1 μg

MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

HIC method: HPLC analysis was carried out under the following measurement conditions:

### Method 1

HPLC system: Waters ACQUITY ARC HPLC System

Detector: measurement wavelength: 280 nm

Column: Tosoh Bioscience 4.6 m ID×3.5 cm, 2.5 m butyl-nonporous resin column

Column temperature: 25° C.

Mobile phase A: 1.5 M ammonium sulfate, 50 mM phosphate buffer, pH 7.0

Mobile phase B: 50 mM phosphate buffer, 25% (V/V) isopropanol, pH 7.0

Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)

Injected sample amount: 20 g

### Method 2

HPLC system: Waters ACQUITY ARC HPLC System

Detector: measurement wavelength: 280 nm

Column: MABPac HIC-10, 5 m, 4.6×10 mm (Thermo)

Column temperature: 25° C.

Mobile phase A: 1.5 M ammonium sulfate, 50 mM sodium phosphate, pH 7.0

Mobile phase B: 50 mM sodium phosphate, pH 7.0

Gradient program: 20% B-20% B (0 min-1 min), 0% B-0% B (1 min-35 min), 20% B-20% B (35 min-40 min)

Flow rate: 0.5 mL/min

Sample preparation: The sample was diluted with initial mobile phase to 0.5 mg/mL.

SEC method to determine ADC purity: HPLC analysis was carried out under the following measurement conditions:

HPLC system: Waters H-Class UPLC System

Detector: measurement wavelength: 280 nm

Column: ACQUITY UPLC BEH200 SEC 1.7 um 4.6×150 mm, Waters

Column temperature: room temperature

Mobile phase A: 200 mM phosphate buffer, 250 mM potassium chloride, 15% isopropyl alcohol, pH 7.0

Gradient program: under 10 min isocratic elutions with the flow rate of 0.3 mL/min

Injected sample amount: 20 g

ADC hydrophobicity evaluation: ADC hydrophobicity was evaluated using the HIC (hydrophobicity interaction column) chromatography method described above. An ADC with a higher hydrophobic property would appear with a later retention time from HIC. Results are presented in Table 4 using the DAR8 peak as a reference

TABLE 1
Conjugators

| Compound No. | Conjugator Structure |
|---|---|
| 1-1 | 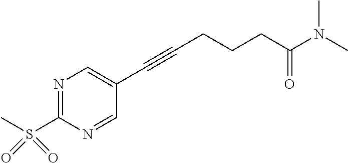 |
| 1-2 | 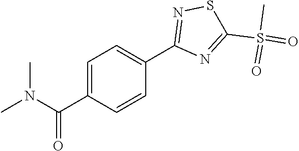 [SMILES: CN(C)C(=O)c1ccc(-c2nsc(S(C)(=O)=O)n2)cc1] |
| 1-3 | 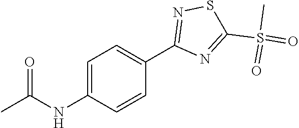 [SMILES: CC(=O)Nc1ccc(-c2nsc(S(C)(=O)=O)n2)cc1] |
| 1-4 | 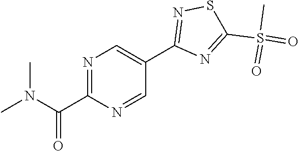 [SMILES: CN(C)C(=O)c1ncc(-c2nsc(S(C)(=O)=O)n2)cn1] |
| 1-5 | 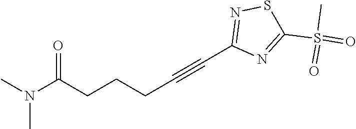 [SMILES: CN(C)C(=O)CCCC#Cc1nsc(S(C)(=O)=O)n1] |

TABLE 2
Conjugator-linker-payloads

| Com- | |
|---|---|
| pound | |
| No. | Conjugator-Linker-Payload Structure |
| 2-1 | 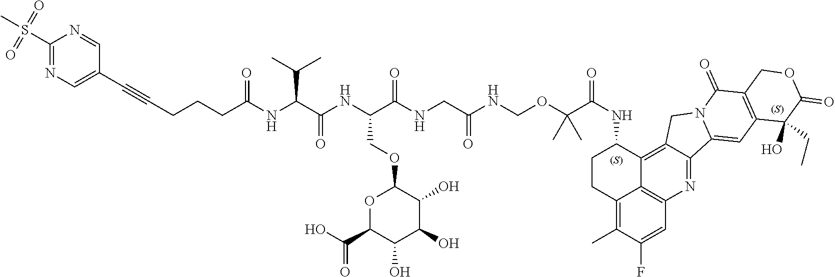 |
| 2-2 | 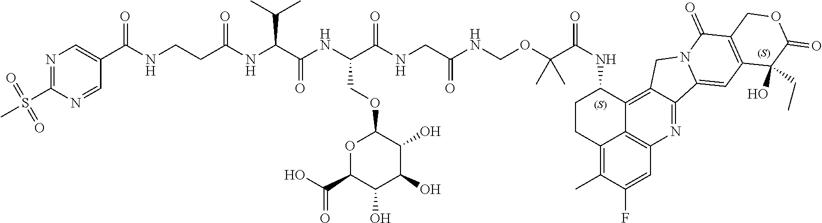 |
| 2-3 | 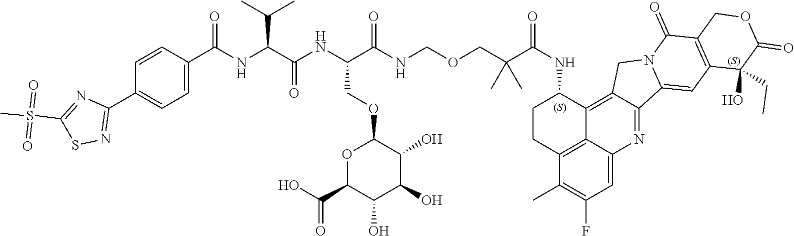 |
| 2-4 | 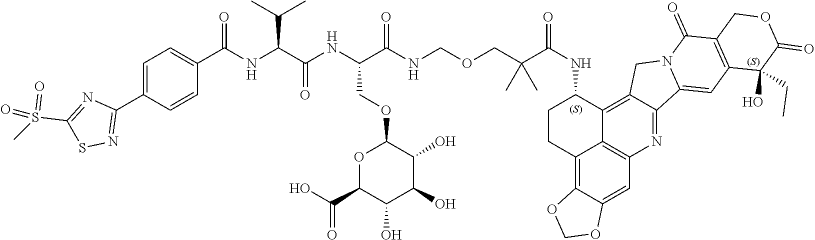 |
| 2-5 | 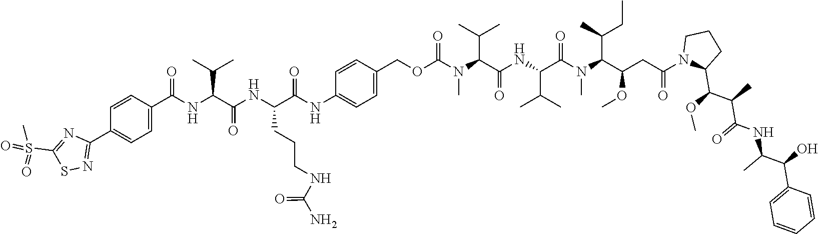 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2ccc(-c3nsc(S(C)(=O)=O)n3)cc2)C(C)C)cc1)C(C)C] |
| 2-6 | 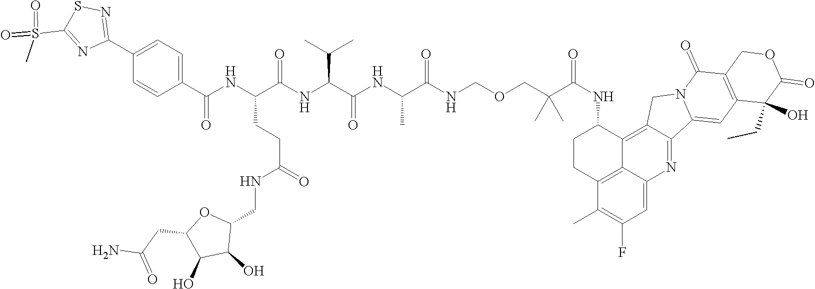 [SMILES: CC[C@@]1(O)C(=O)OCc2c1cc1n(c2=O)Cc2c-1nc1cc(F)c(C)c3c1c2[C@@H](NC(=O)C(C)(C)COCNC(=O)[C@H](C)NC(=O)[C@@H](NC(=O)[C@H](CCC(=O)NC[C@H]1O[C@@H](CC(N)=O)[C@H](O)[C@@H]1O)NC(=O)c1ccc(-c2nsc(S(C)(=O)=O)n2)cc1)C(C)C)CC3] |

TABLE 3
Conjugator-antibody conjugates

| Conjugate-Antibody | | | |
|---|---|---|---|
| Conjugate No. | Antibody | Conjugator Structure | MS DAR |
| 3-1 | Herceptin | 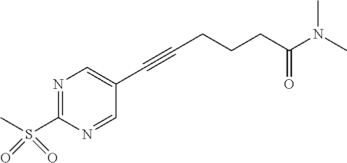 | 8.00 |
| 3-2 | Herceptin | 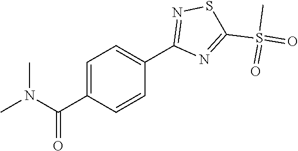 [SMILES: CN(C)C(=O)c1ccc(-c2nsc(S(C)(=O)=O)n2)cc1] | 8.62 |
| 3-3 | Herceptin | 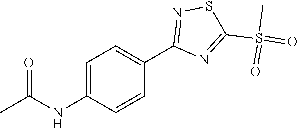 [SMILES: CC(=O)Nc1ccc(-c2nsc(S(C)(=O)=O)n2)cc1] | |
| 3-4 | Herceptin | 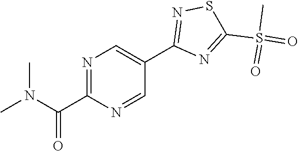 [SMILES: CN(C)C(=O)c1ncc(-c2nsc(S(C)(=O)=O)n2)cn1] | |
| 3-5 | Herceptin | 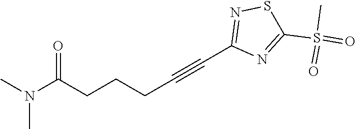 [SMILES: CN(C)C(=O)CCCC#Cc1nsc(S(C)(=O)=O)n1] | |

TABLE 4
ADCs

| | | | DAR8 Species |
|---|---|---|---|
| ADC | | | HIC Retention |
| No. | Antibody | ADC Structure | Time (min) (Method 1) |
| 4-A | 6E7 | 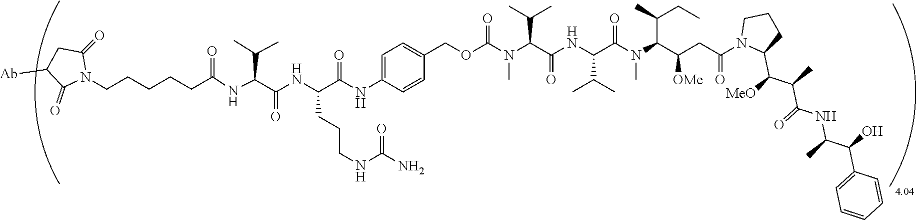 | 14.466 |
| 4-B | Ifinatamab | 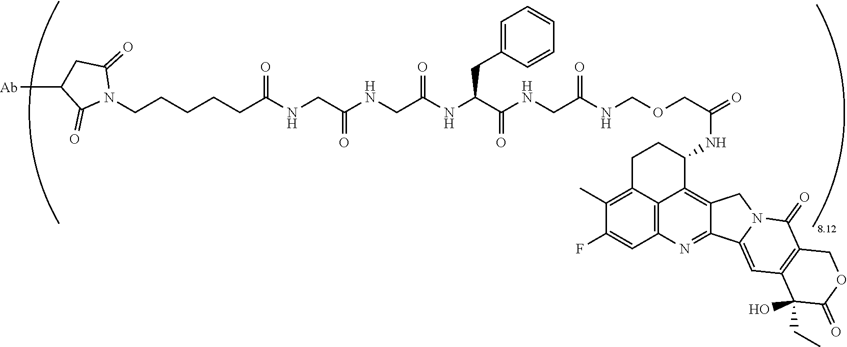 | 10.860 |
| 4-1 | Ifinatamab | 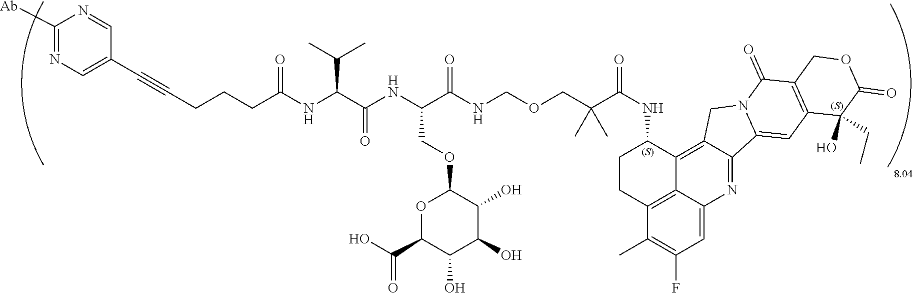 | 9.765 |
| 4-2 | Ifinatamab | 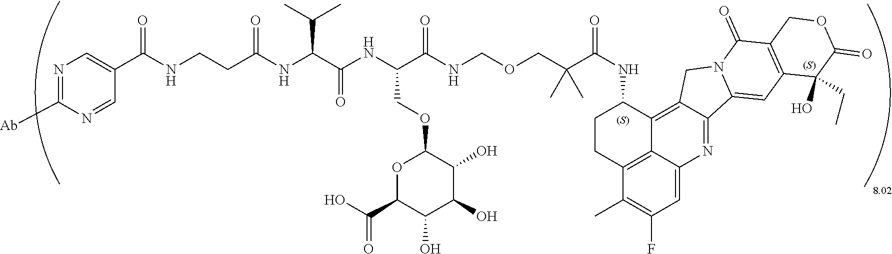 | 9.704 |
| 4-3 | Ifinatamab | 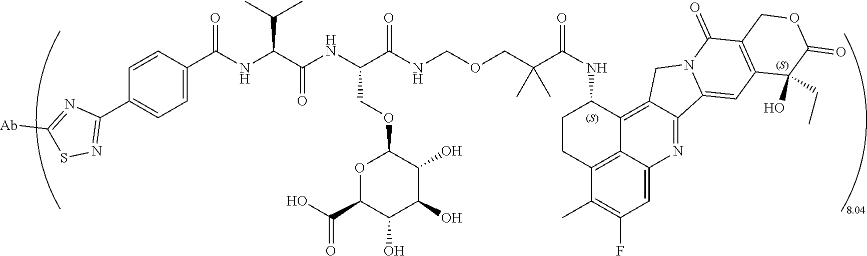 | 9.756 |
| 4-A | Ifinatamab | 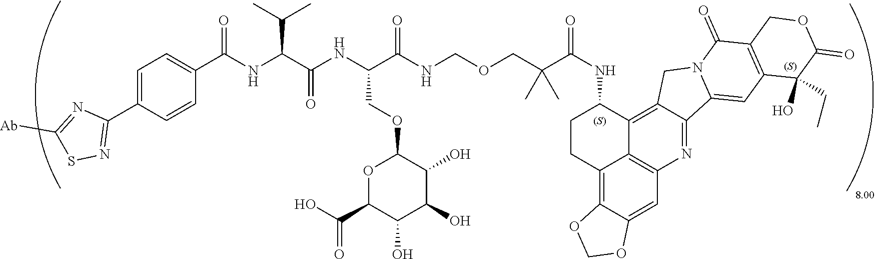 | 9.487 |
| 4-5 | 6E7 | 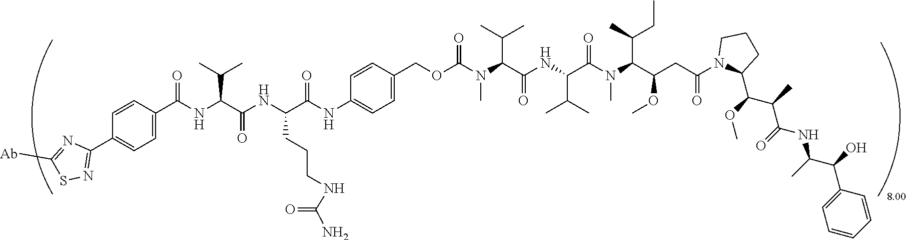 | 14.167 |
| 4-6 | Ifinatamab | 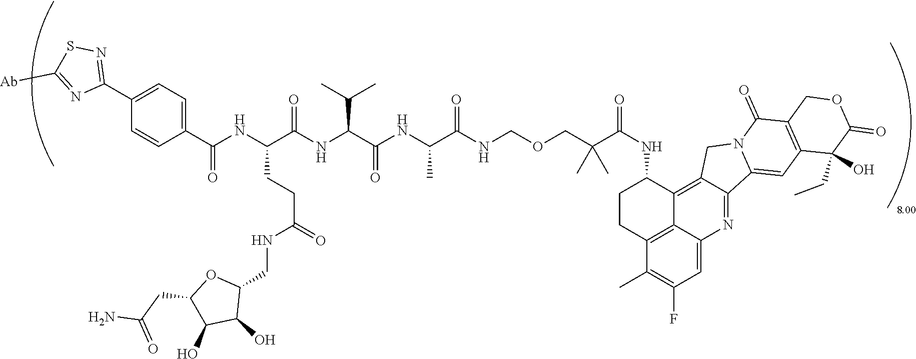 | 9.217 |

### Antibody Information

Ifinatamab (anti-B7H3 antibody)
Light Chain sequence
(SEQ ID NO: 1)
EIVLTQSPATLSLSPGERATLSCRASSRLIYMHWYQQKPGQAPRP
LIYATSNLASGIPARFSGSGSGTDFTLTISSLEPEDFAVYYCQQW
NSNPPTFGQGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC
Heavy Chain sequence
(SEQ ID NO: 2)
QVQLVQSGAEVKKPGSSVKVSCKASGYTFTNYVMHWVRQAPGQGL
EWMGYINPYNDDVKYNEKFKGRVTITADESTSTAYMELSSLRSED
TAVYYCARWGYYGSPLYYFDYWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKRVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVMHEALHNHYTQKSLSLSP
GK
6E7 (anti-CLL antibody)
Light Chain sequence
(SEQ ID NO: 3)
DIQMTQSPSSLSASVGDRVTITCRASQSVSTSSYNYMHWYQQKPG
KPPKLLIKYASNLESGVPSRFSGSGSGTDFTLTISSLQPEDFATY
YCQHSWEIPLTFGQGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Heavy Chain sequence
(SEQ ID NO: 4)
EVQLVQSGAEVKKPGASVKVSCKASGYSFTDYYMHWVRQAPGQGL
EWIGRINPYAGAAFYSQNFKDRVTLTVDTSTSTAYLELSSLRSED
TAVYYCAIERGADLEGYAMDYWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GK

### Conjugator-Antibody Conjugates Stability Study: ADC Storage Buffer and GSH Solution

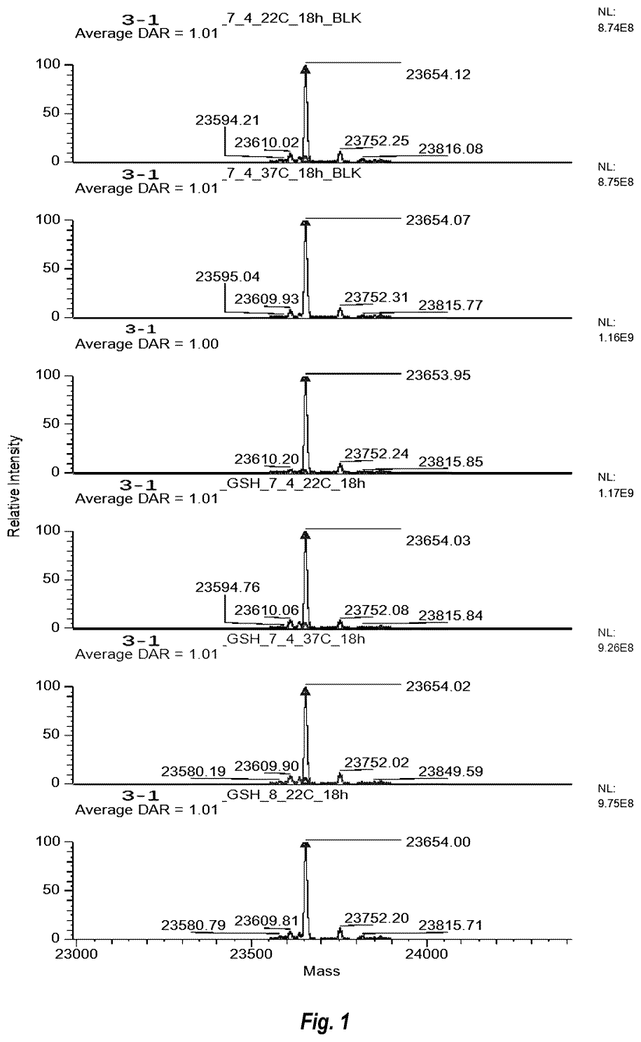

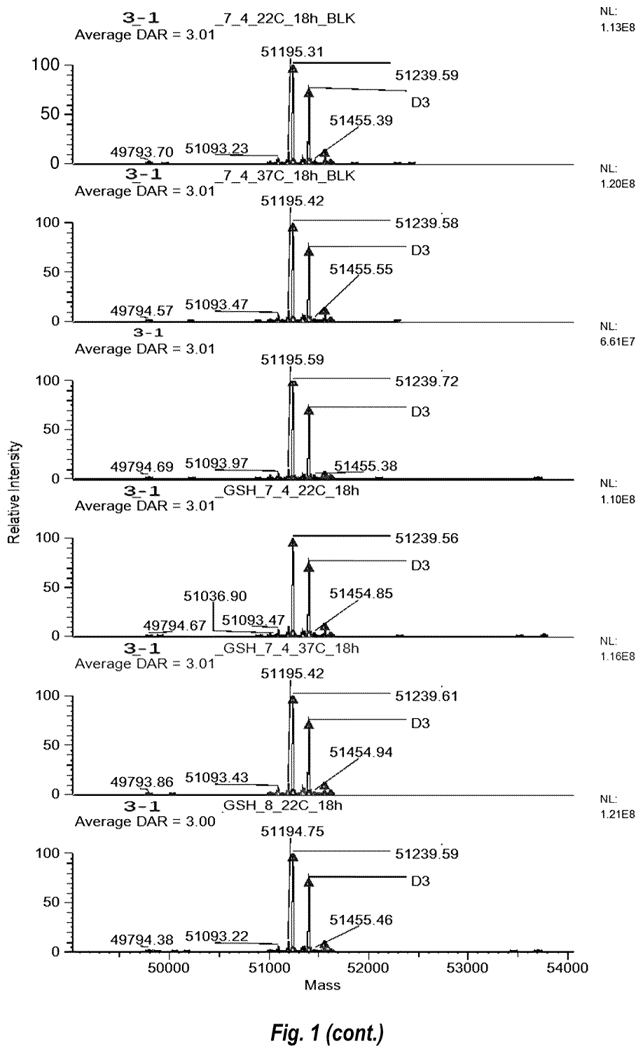

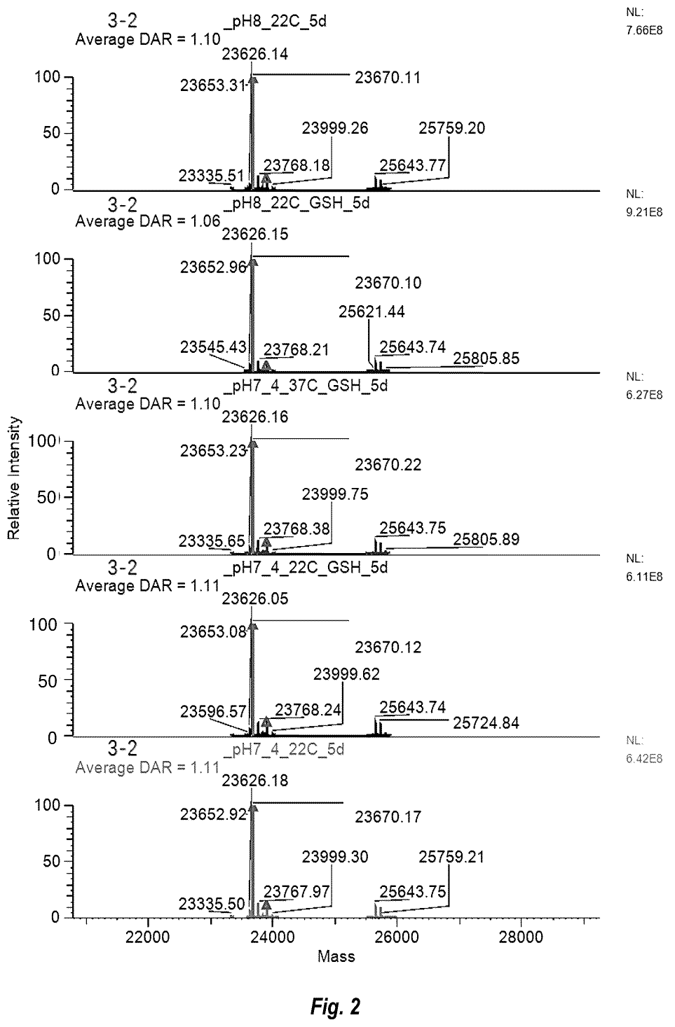

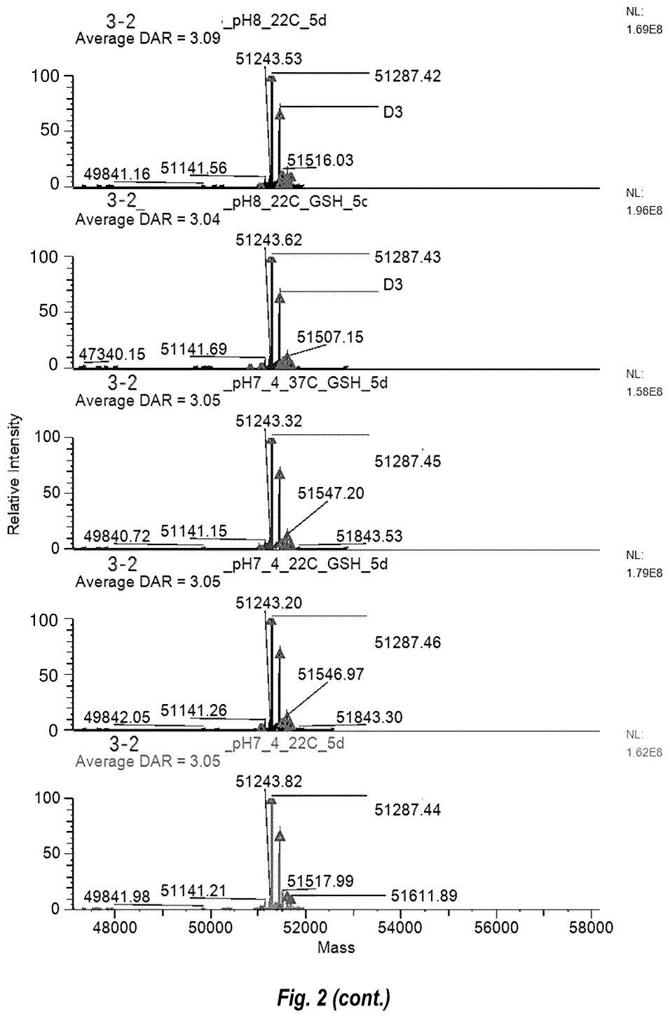

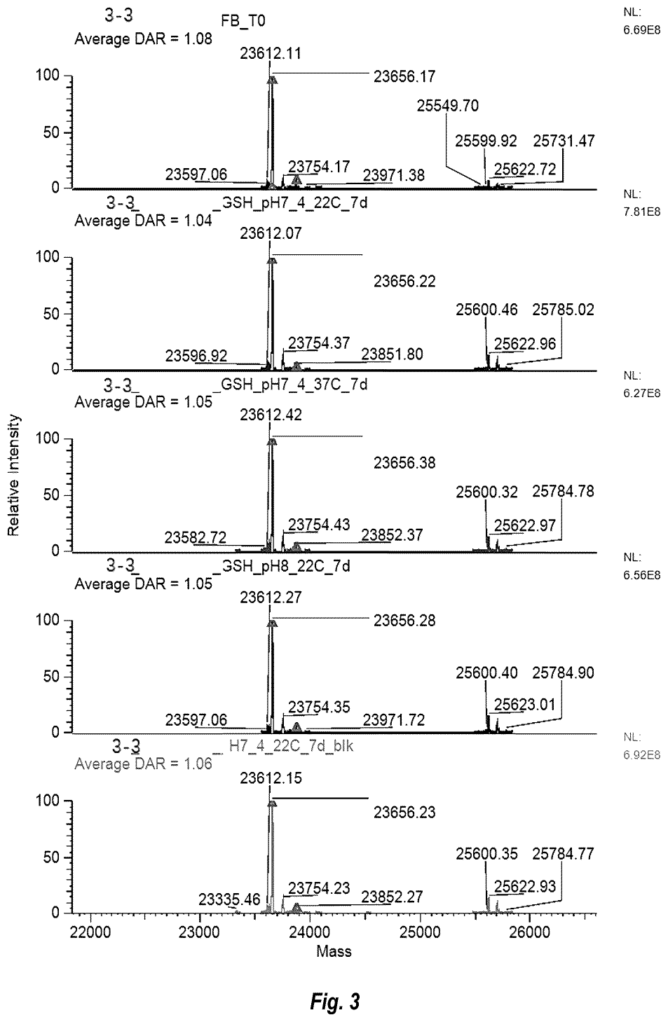

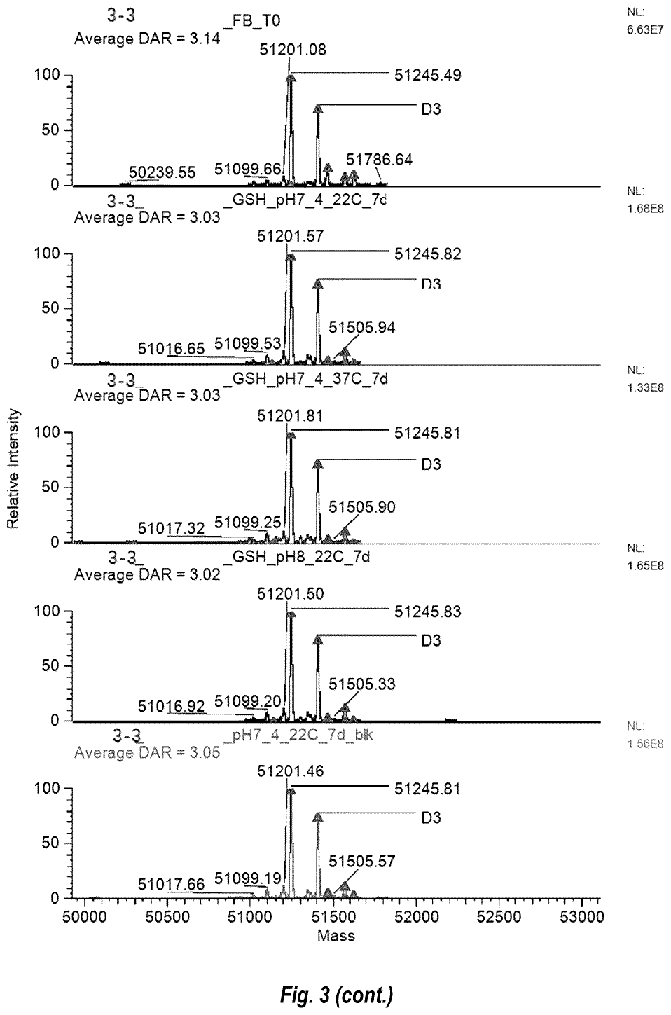

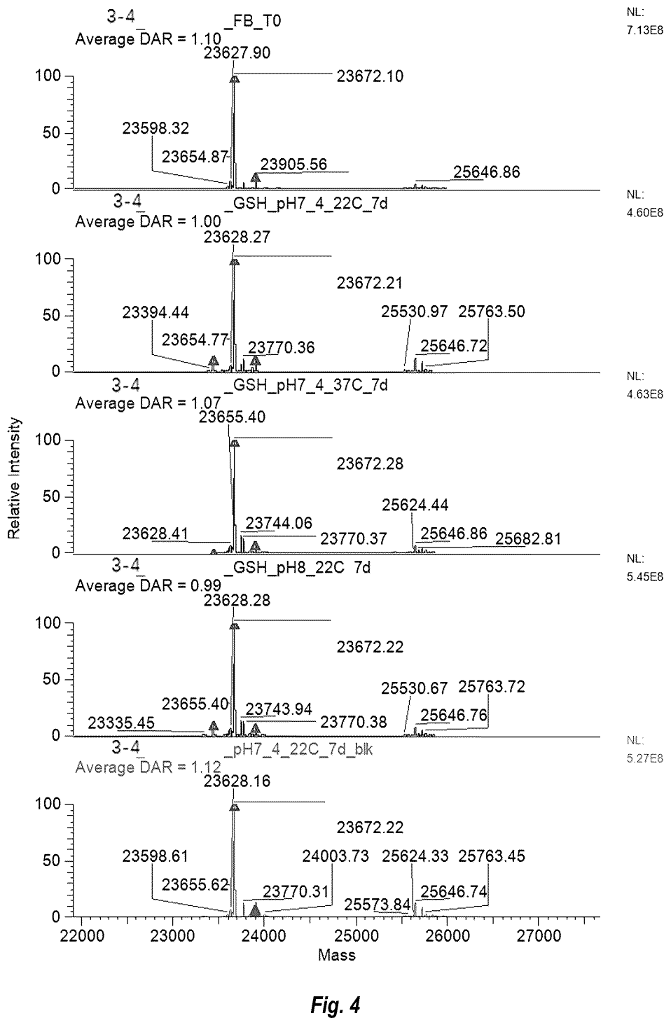

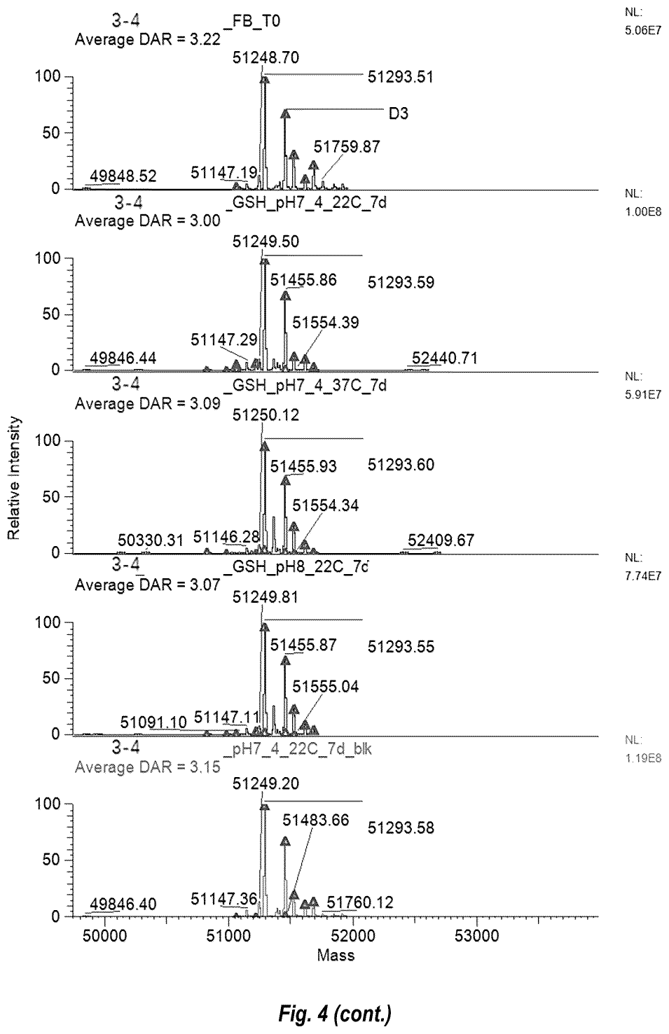

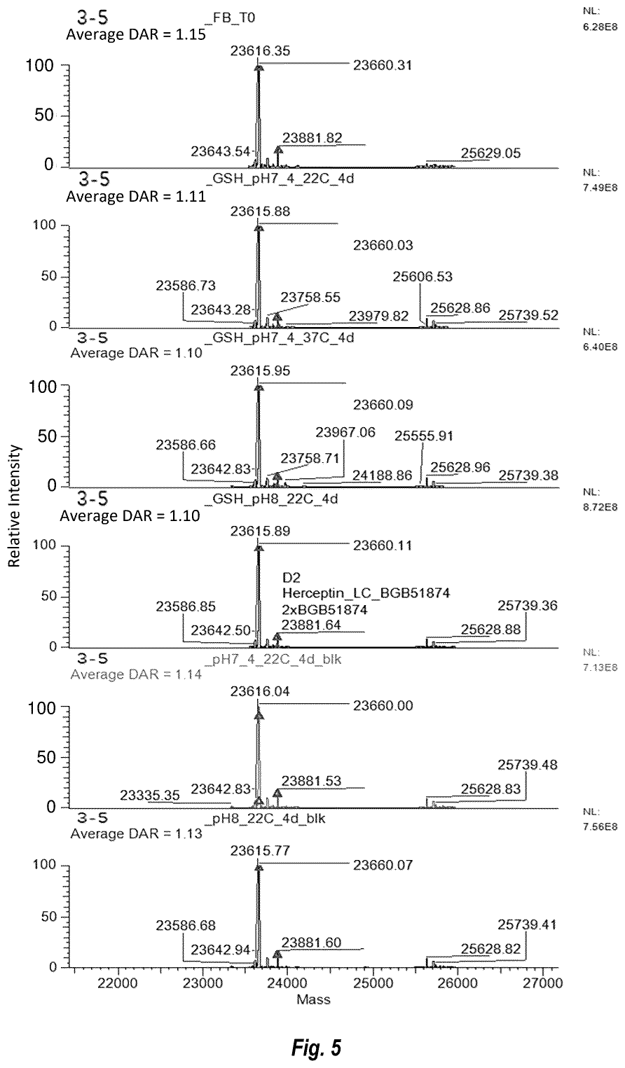

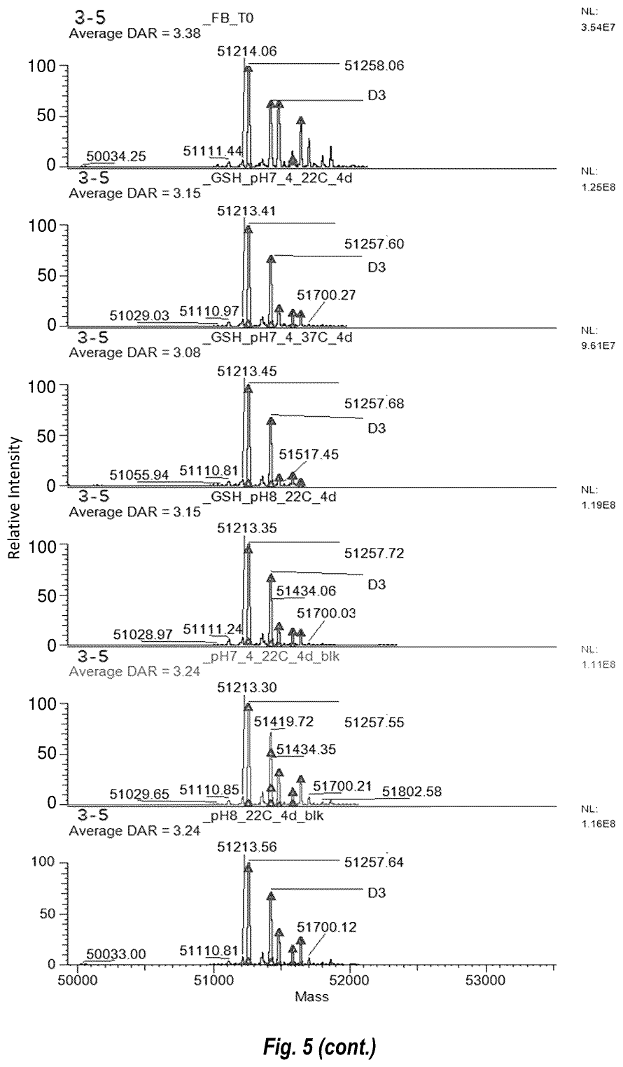

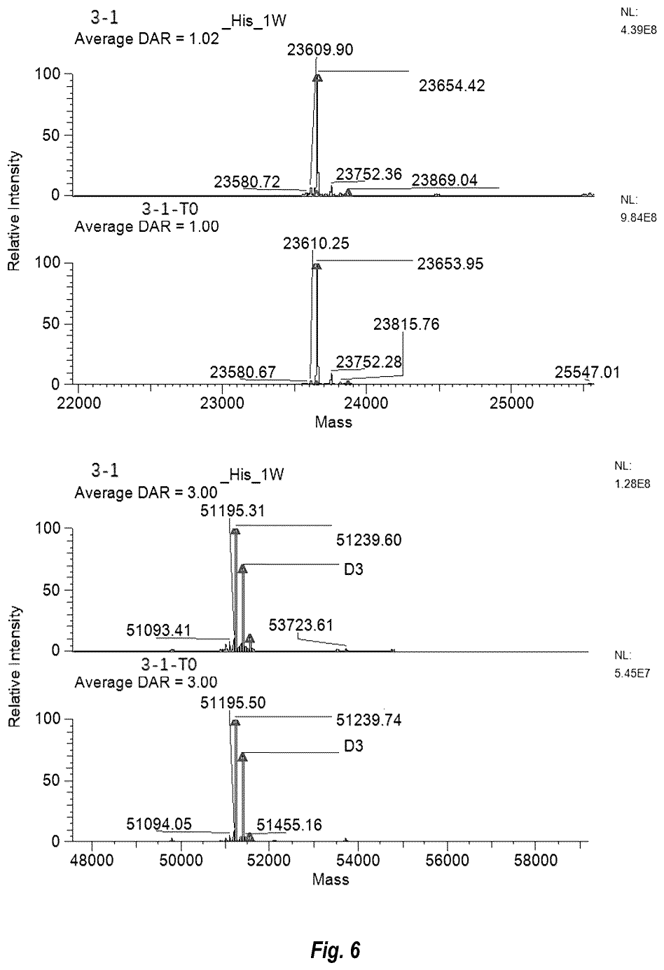

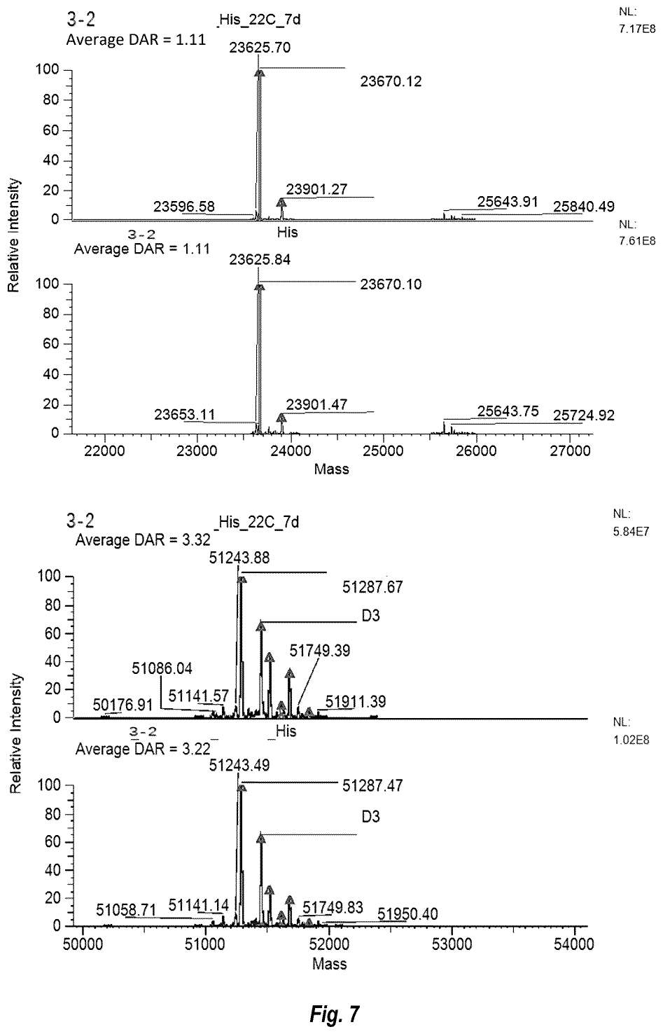

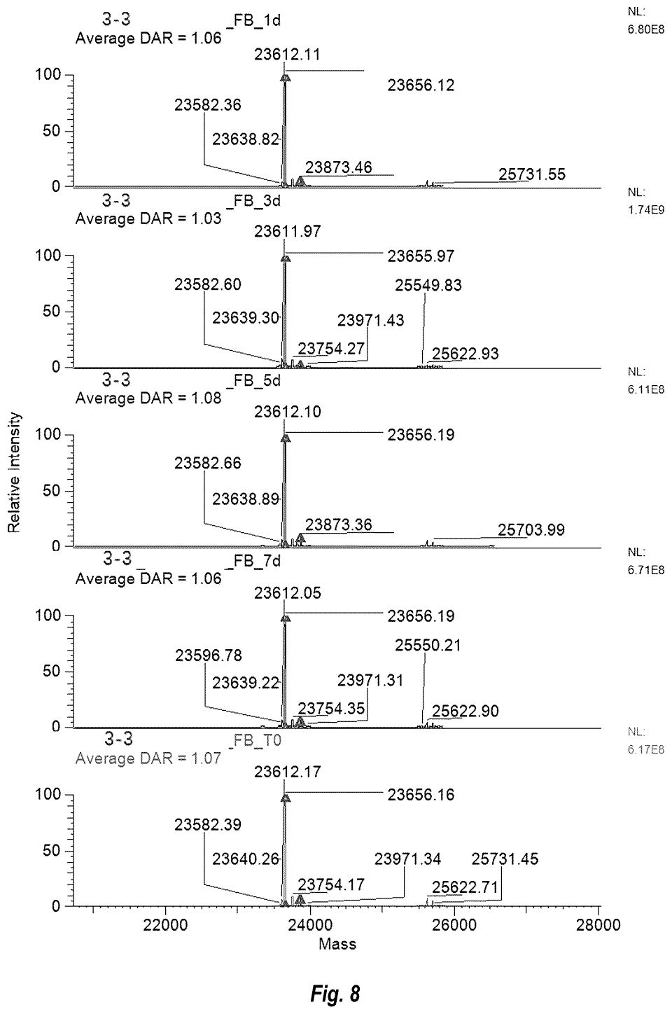

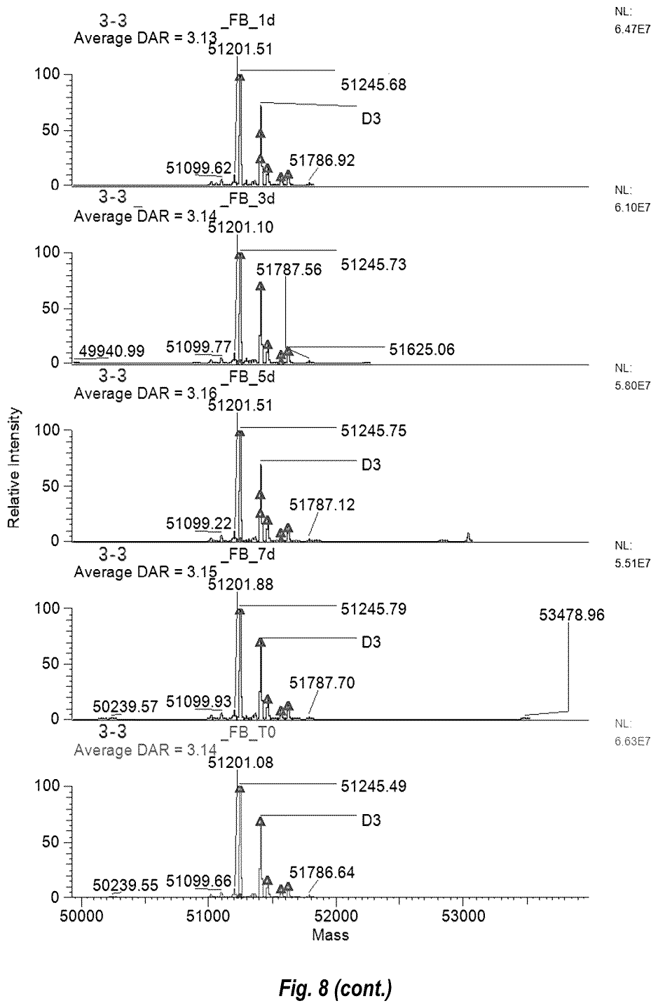

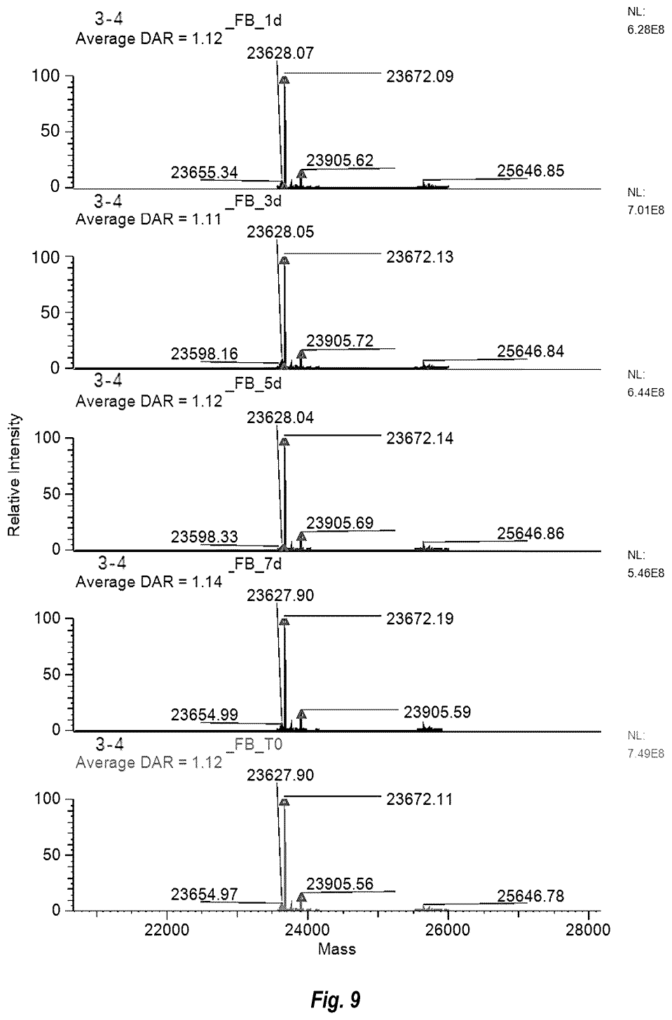

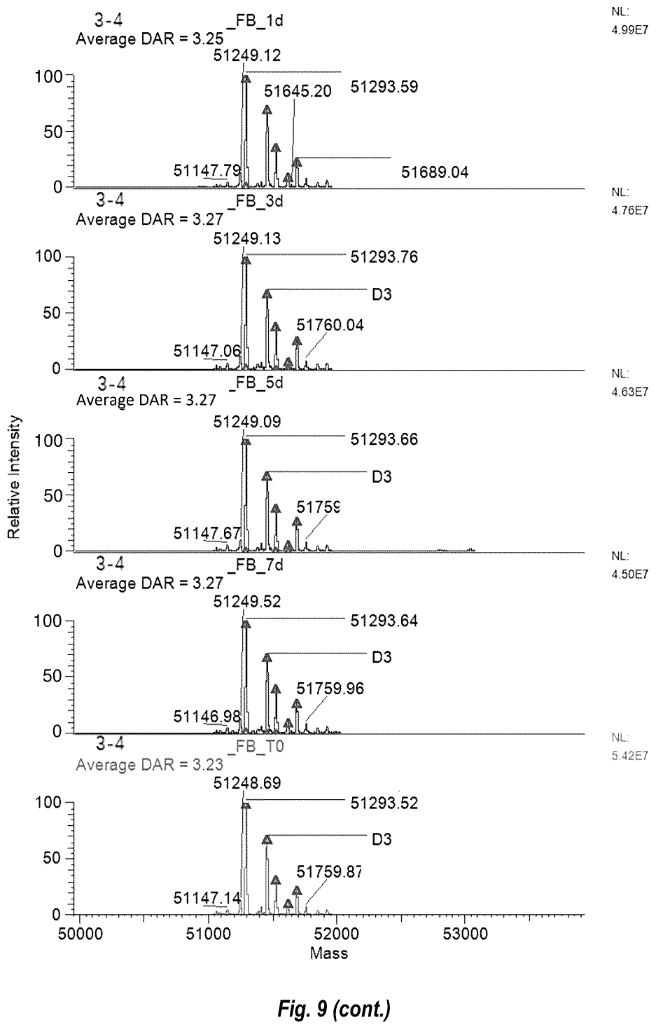

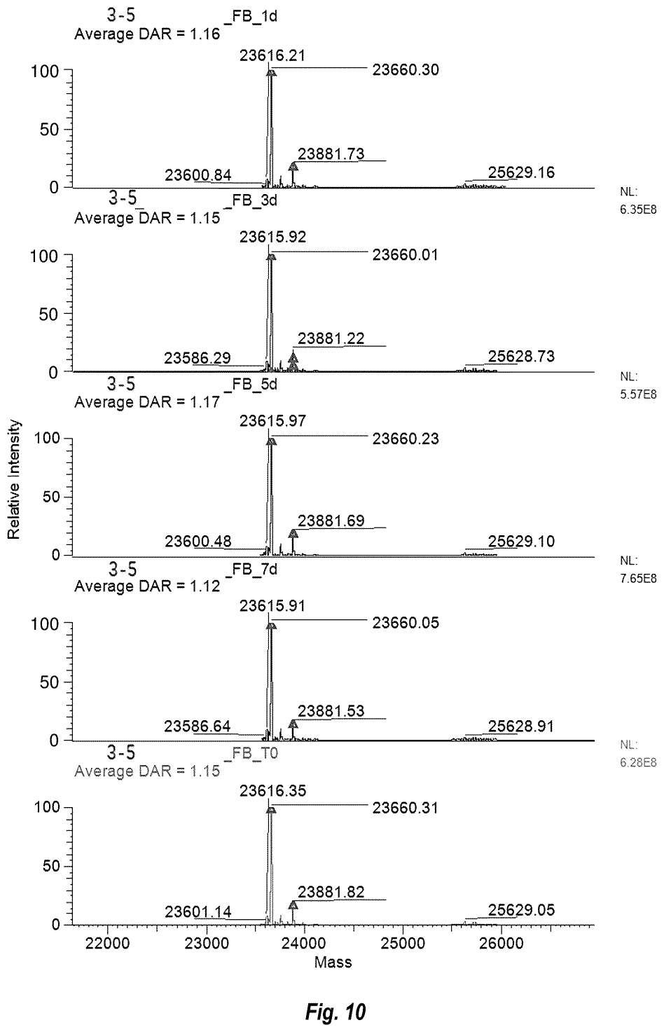

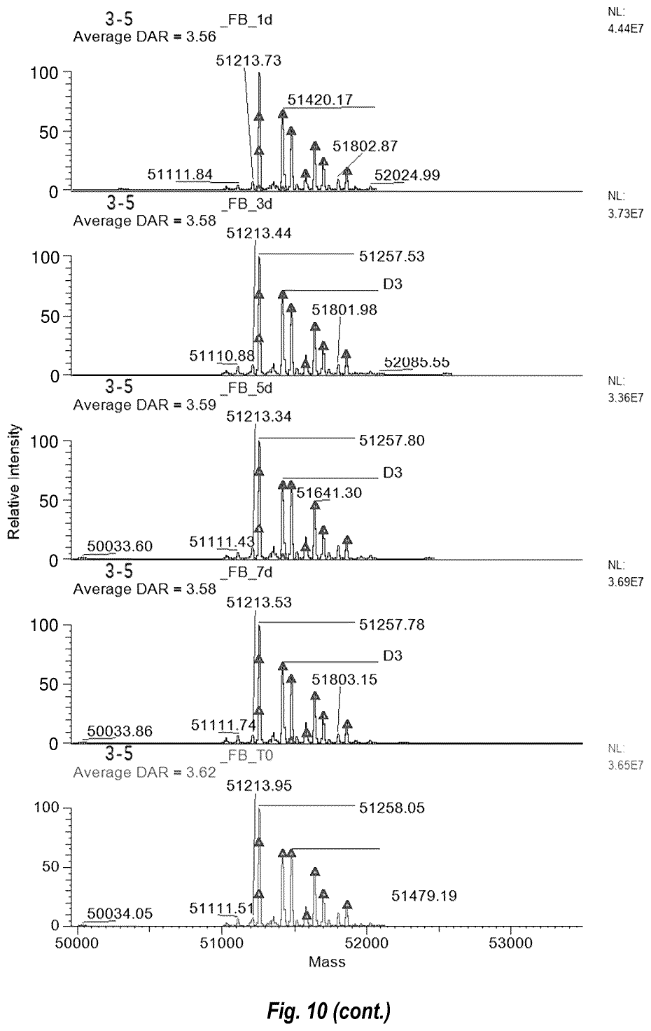

In these investigations, the stabilities of conjugator-antibody conjugates in a typical ADC acidic storage buffer were investigated, as were deconjugation events when the conjugates were incubated in glutathione (GSH) solutions. Specifically, after the conjugation steps described above (see “DAR8 antibody drug conjugate/conjugator-antibody conjugate preparation”), conjugator-antibody conjugates were incubated in formulation buffer (pH 5.5 20 mM histidine buffer) or GSH buffer (pH 7.4 or 8.0), and the solutions were left at 22 or 37° C. for 1˜168 h. Results are presented in Tables 5 and 6 and FIGS. 1-10).

Stability with GSH

| Conjugate (mM) | GSH (mM) | pH | Time (h) | Temp. (° C.) |
|---|---|---|---|---|
| 0.001-0.03 | 10 | 7.4/8.0 | 18 | 22/37 |

TABLE 5
Conjugator-antibody stability in GSH buffer (pH 7.4 and 8.0)

| Conjugator- | | | | | MS DAR |
|---|---|---|---|---|---|
| Antibody | Incubation | | | T0 | after |
| Conjugate No. | Time (h) | pH | Temp. (° C.) | MS DAR | incubation |
| 3-1 | 18 | 7.4 | 22 | 8.00 | 8.04 |
| | | | 37 | 8.00 | 8.04 |
| | | 8.0 | 22 | 8.00 | 8.02 |
| 3-2 | 120 | 7.4 | 22 | 8.66 | 8.32 |
| | | | 37 | 8.66 | 8.30 |
| | | 8.0 | 22 | 8.66 | 8.20 |
| 3-3 | 168 | 7.4 | 22 | 8.42 | 8.14 |
| | | | 37 | 8.42 | 8.16 |
| | | 8.0 | 22 | 8.42 | 8.14 |
| 3-4 | 168 | 7.4 | 22 | 8.70 | 8.00 |
| | | | 37 | 8.70 | 8.32 |
| | | 8.0 | 22 | 8.70 | 8.12 |
| 3-5 | 96 | 7.4 | 22 | 9.54 | 8.52 |
| | | | 37 | 9.54 | 8.36 |
| | | 8.0 | 22 | 9.54 | 8.50 |

The results demonstrate that DAR values of all conjugates remained almost the same as TO after incubation in GSH buffers (pH 7.4 at 22/37° C. and pH 8.0 at 22° C.) for at least 18 h.

TABLE 6
Conjugator-antibody stability in formulation
buffer (pH 5.5 20 mM histidine buffer)

| Conjugator- | | | | |
|---|---|---|---|---|
| Antibody | | | | |
| Conjugate | Incubation | Temp. | | MS DAR-T = |
| No. | Time (h) | (° C.) | MS DAR-T0 | 168 h |
| 3-1 | 168 | 22 | 8.00 | 8.04 |
| 3-2 | 168 | 22 | 8.66 | 8.86 |
| 3-3 | 168 | 22 | 8.42 | 8.42 |
| 3-4 | 168 | 22 | 8.70 | 8.82 |
| 3-5 | 168 | 22 | 9.54 | 9.40 |

The results demonstrate that DAR values of all conjugates remained almost the same as TO after incubation in formulation buffer for 168 h.

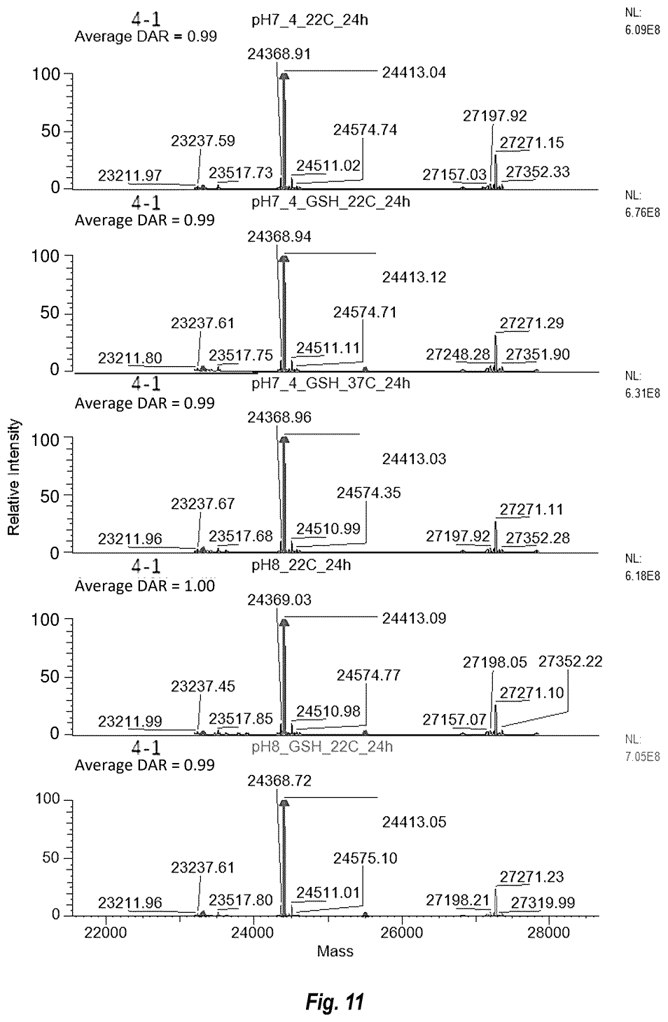

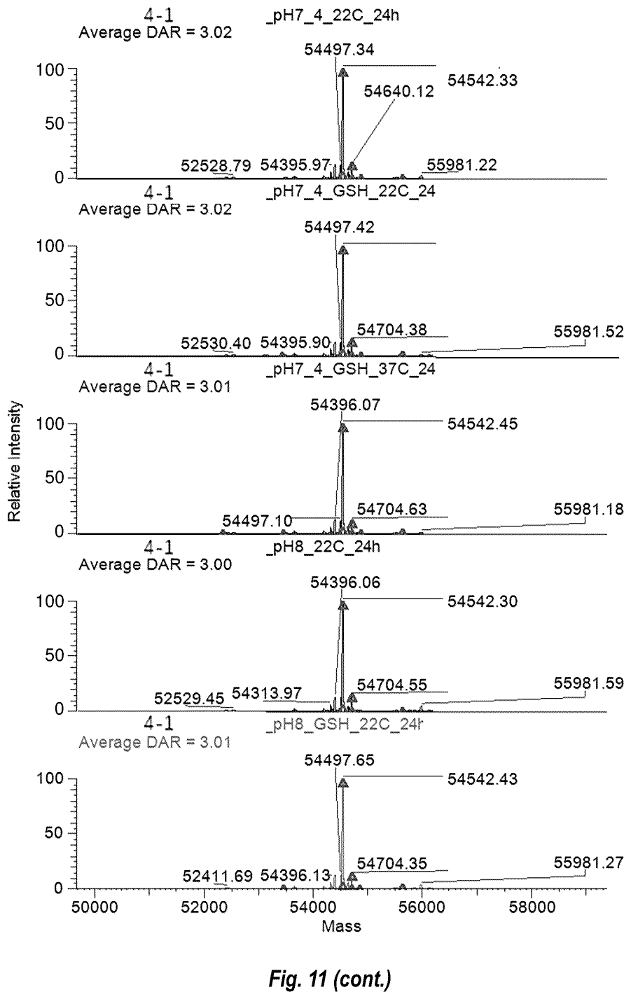

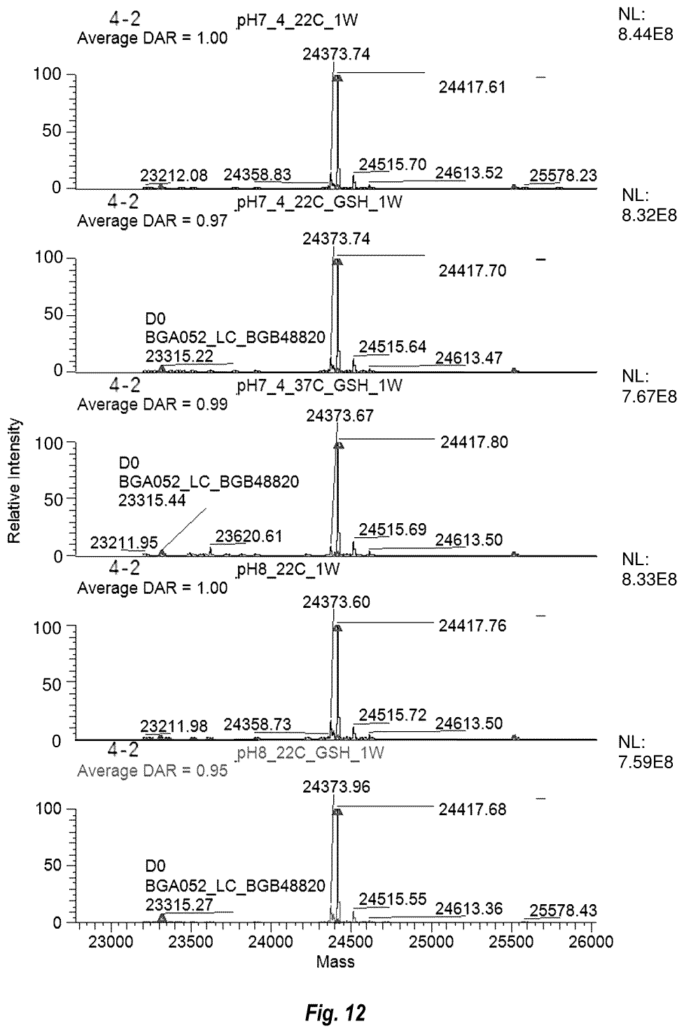

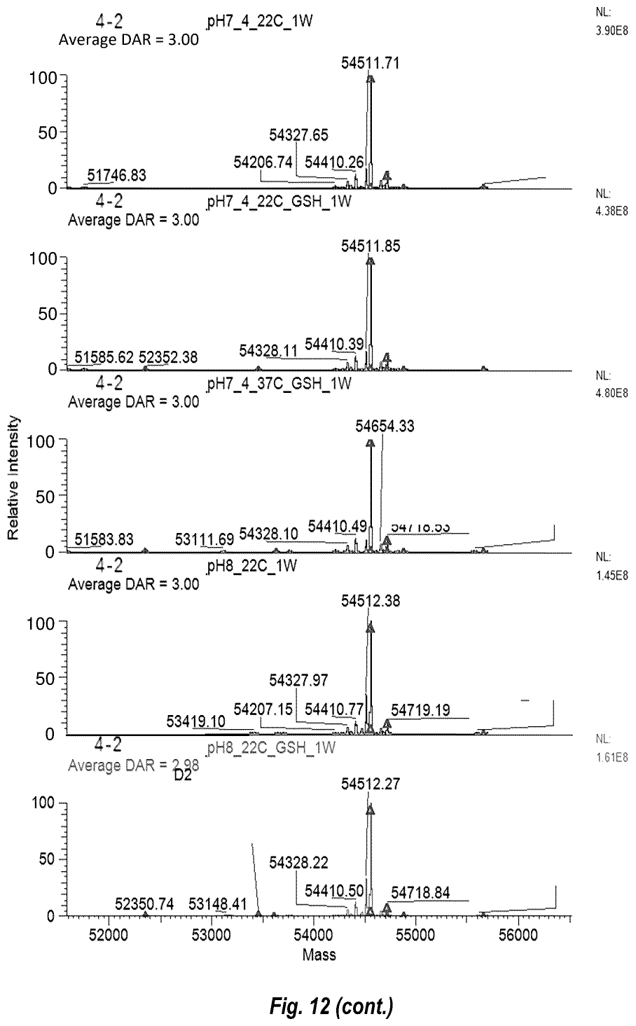

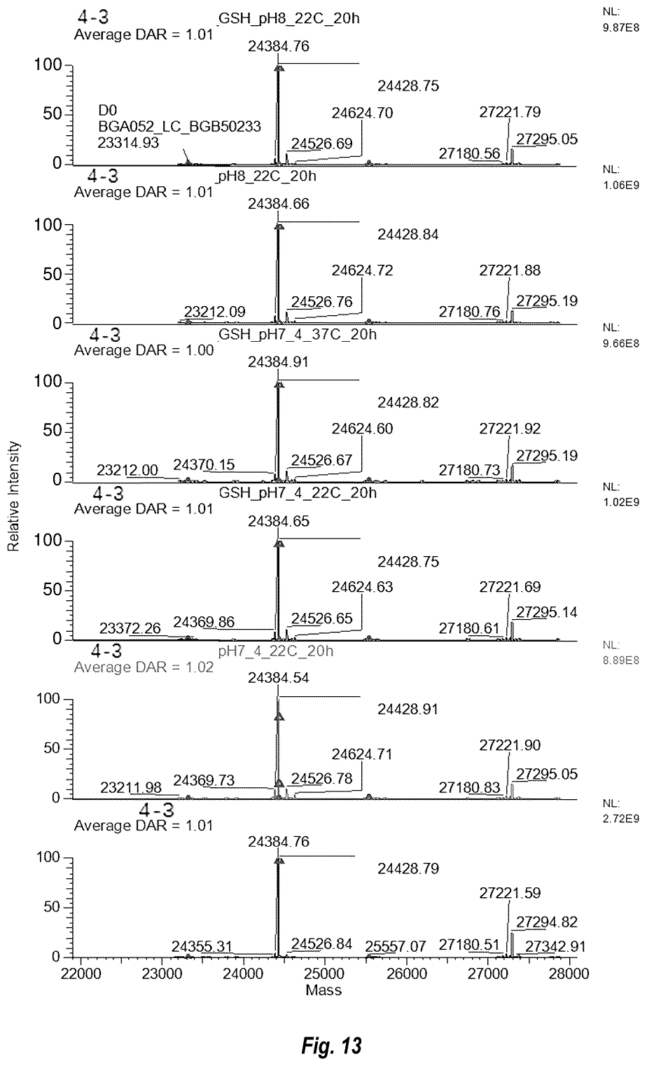

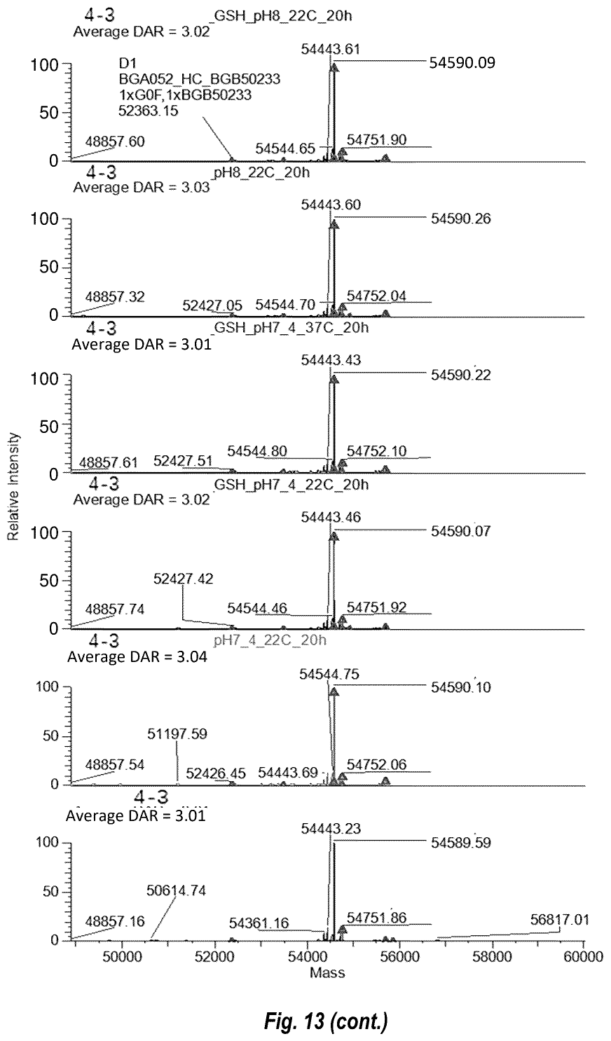

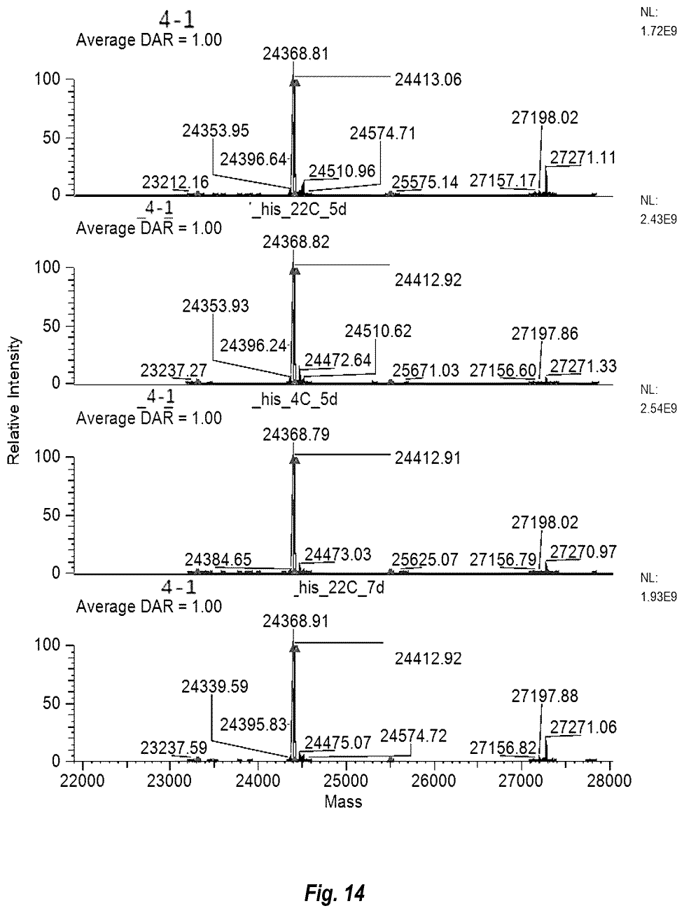

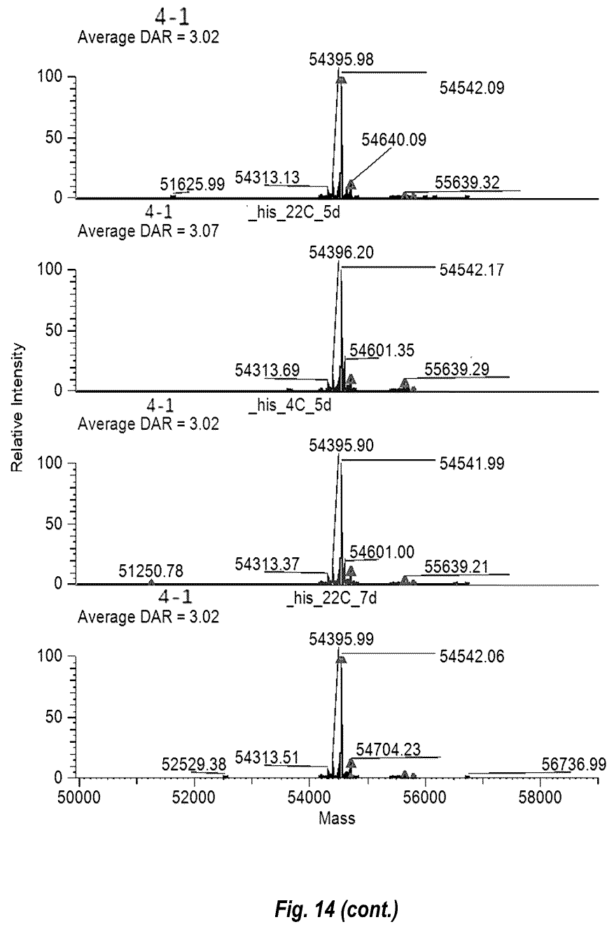

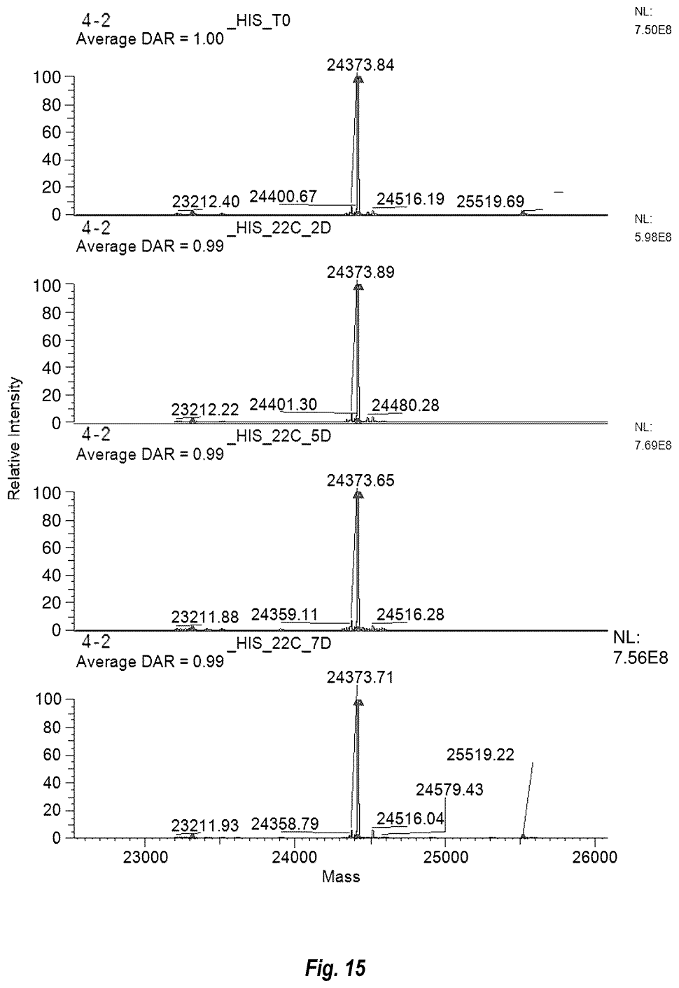

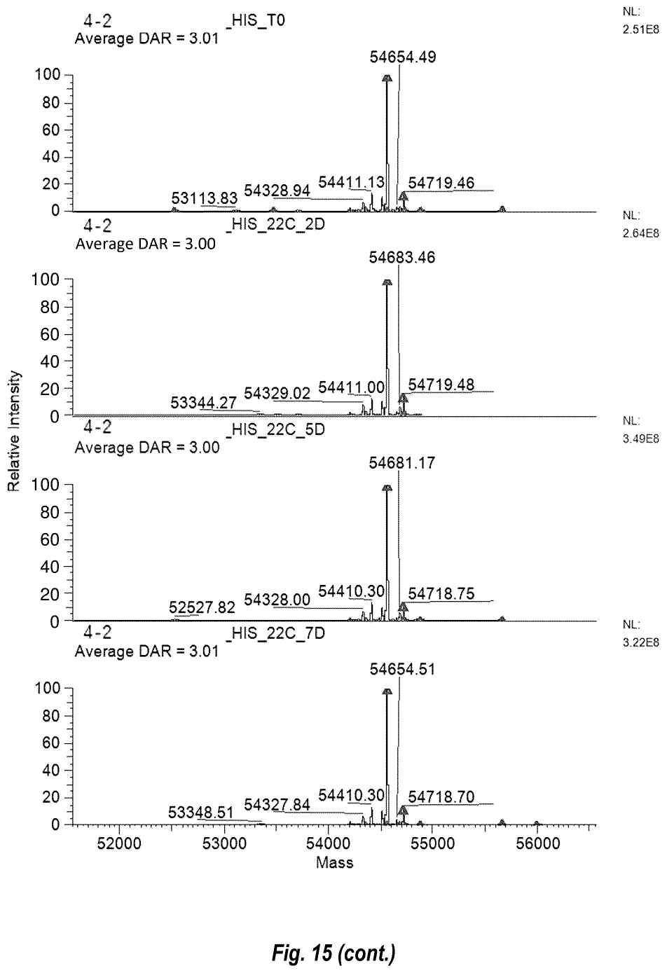

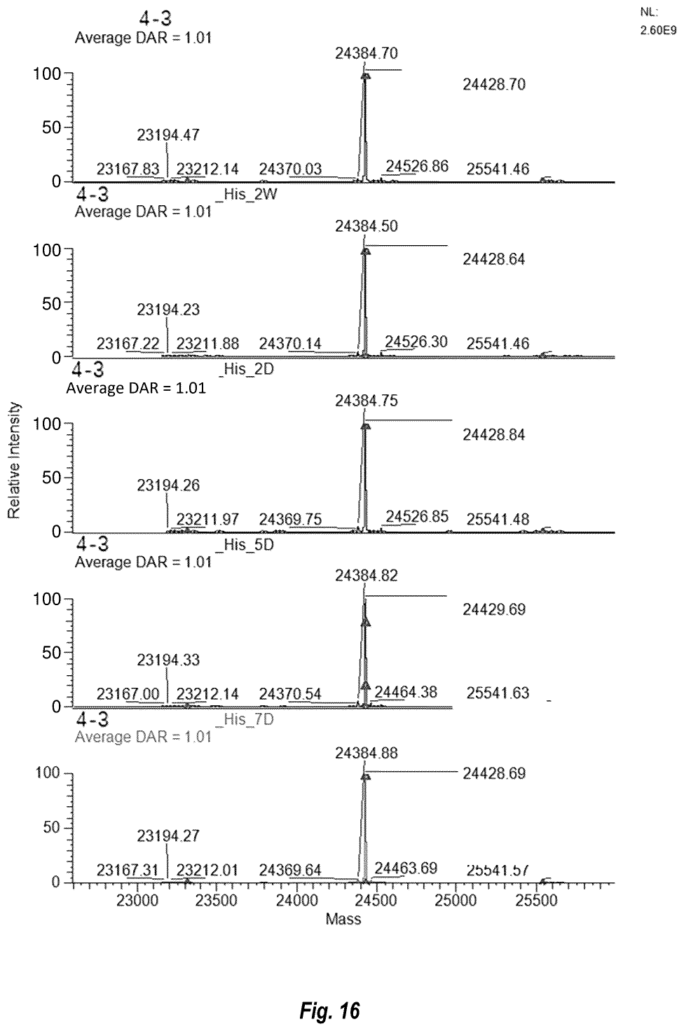

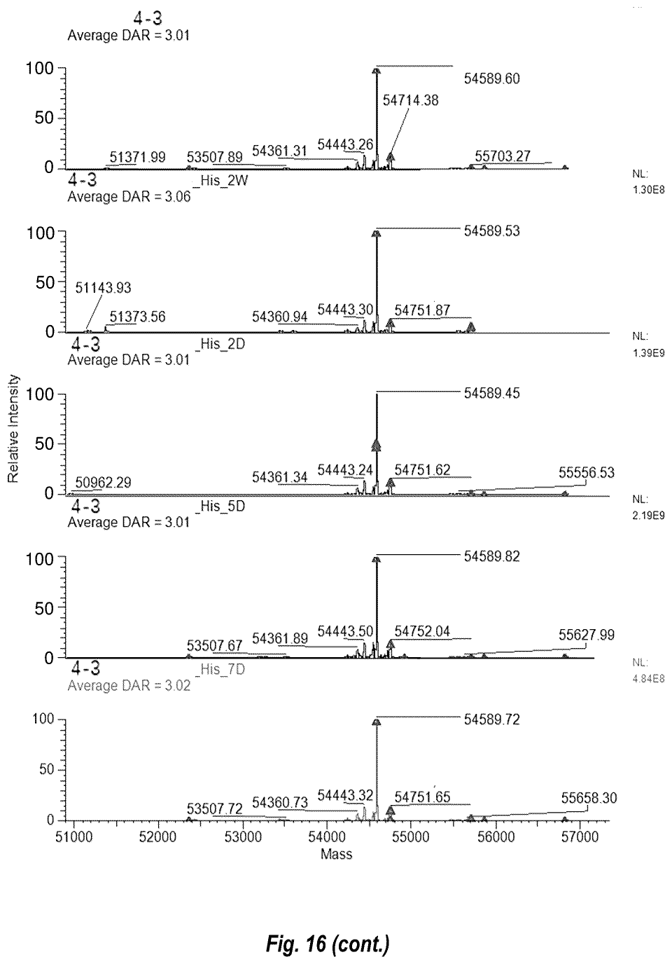

Results of stability assessments of ADCs in GSH solution or formulation buffer are presented in Tables 7 and 8, respectively, and FIGS. 11-16.

TABLE 7
ADC stability in GSH buffer (pH 7.4 and 8.0)

| ADC | Incubation | | Temp. | T0 | MS DAR after |
|---|---|---|---|---|---|
| No. | Time (h) | pH | (° C.) | MS DAR | incubation |
| 4-1 | 24 | 7.4 | 22 | 8.04 | 8.02 |
| | | | 37 | 8.04 | 8.00 |
| | | 8.0 | 22 | 8.04 | 8.00 |
| 4-2 | 168 | 7.4 | 22 | 8.02 | 7.94 |
| | | | 37 | 8.02 | 7.98 |
| | | 8.0 | 22 | 8.02 | 7.86 |
| 4-3 | 20 | 7.4 | 22 | 8.04 | 8.06 |
| | | | 37 | 8.04 | 8.02 |
| | | 8.0 | 22 | 8.04 | 8.06 |

The results presented in Table 7 demonstrate that no deconjugation events were observed for ADC 4-1 to 4-3 after incubation in GSH buffers (pH 7.4 at 22/37° C. and pH 8.0 at 22° C.).

TABLE 8
ADC stability in formulation buffer
(pH 5.5 20 mM histidine buffer)

| ADC | Incubation | Temp. | | MS DAR-T = |
|---|---|---|---|---|
| No. | Time (h) | (° C.) | MS DAR-T0 | 168 h |
| 4-1 | 168 | 22 | 8.04 | 8.04 |
| 4-2 | 168 | 22 | 8.02 | 8.00 |
| 4-3 | 168 | 22 | 8.04 | 8.06 |

The results presented in Table 8 demonstrate that no deconjugation events were observed for ADC 4-1 to 4-3 after storage in formulation buffer for one week.

### Cell Lines

NCI-H11650 (ATCC, CRL-5883). NCI-H1650 is a cell line exhibiting epithelial morphology that was isolated in 1987 from the lung tissue of a 27-year-old male smoker with stage 3, bronchoalveolar carcinoma, and NCI-H1650 was purchased from ATCC. The base medium for NCI-H1650 is ATCC-formulated RPMI-1640 Medium, ATCC 30-2001. To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

Capan-1 (ATCC, HTB-79). Capan-1 is a cell line with epithelial morphology that was isolated from the pancreas of a 40-year-old white male with pancreatic adenocarcinoma, and Capan-1 was purchased from ATCC. The base medium for Capan-1 is ATCC-formulated Iscove's Modified Dulbecco's Medium, Catalog No. 30-2005. To make the complete growth medium, fetal bovine serum to a final concentration of 20% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

MDA-MB-453 (SIBS). MDA-MB-453 was derived from an effusion of a 48-year-old female patient with metastatic carcinoma of the breast, involving the nodes, brain, and both pleural and pericardial cavities, and MDA-MB-453 was purchased from SIBS. The base medium for MDA-MB-453 is RPMI 1640 Medium, HEPES (Gibco, 22400105). To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

U937 (ATCC, CRL-1593.2). U-937 is a cell line exhibiting monocyte morphology that was derived in 1974 from malignant cells obtained from the pleural effusion of a 37-year-old white male with histiocytic lymphoma. U937 was purchased from ATCC. The base medium for U937 is ATCC-formulated RPMI-1640 Medium (ATCC 30-2001). To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

HL60 (ATCC, CCL-240). HL-60 cells are promyeoloblasts isolated from the peripheral blood by leukopheresis from a 36-year-old white female with acute promyelocytic leukemia. HL60 was purchased from ATCC. The base medium for HL60 is ATCC-formulated Iscove's Modified Dulbecco's Medium, Catalog No. 30-2005. To make the complete growth medium, fetal bovine serum to a final concentration of 20% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

TF1 (ATCC, CRL-2003). TF-1 erythroblasts were isolated in 1987 from bone marrow derived from a 35-year-old Asian male with severe pancytopenia. TF-1 was purchased from ATCC. The base medium for TF-1 is ATCC-formulated RPMI-1640 Medium, Catalog No. 30-2001. To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

NCI-H358 (ATCC, CRL-5807). NCI-H358 cells are epithelial-like cells isolated from the bronchiole of a male patient with bronchioalveolar carcinoma. NCI-H358 was purchased from ATCC. The base medium for NCI-H358 is ATCC-formulated RPMI-1640 Medium, ATCC 30-2001. To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

TABLE 10A
B7H3 expression level

| Cell line | Molecular No. (QSC kit) |
|---|---|
| NCI-H1650 | 505998 |
| Capan-1 | 63062 |
| MDA-MB-453 | 0 |

TABLE 10B
CLL1 expression level

| Cell line | FACS (Emax MFI, 100 nM) | Molecular No. (QSC kit) |
|---|---|---|
| U937 | 1607 | 84858 |
| HL60 | 846 | NA |
| TF-1 | 50 | <5806 |

### ADC Direct Killing in U937, HL60, TF1, NCI-H1650, Capan-1, and MDA-MB-453 Cancer Cell Lines

ADC direct killing was assessed in U937, HL60, and TF1 cancer lines. Cells were seeded (U937 or HL60 (3E3/well) or TF1 (6E3/well)) into 2D 96-well plates (Greiner: 655090), 100 μl/well (including 150 μg/ml Fc blocker), and incubated at 37° C., 5% CO₂, overnight. Fresh growth medium was added containing varying concentrations of ADCs, 50 μl/well, and incubated at 37° C., 5% CO₂, for 6 days. The cell viability was detected by Cell Titer-Glo (Promega, G7573), 70 μl/well. The 2D plates were allowed to incubate at room temperature for 10 minutes to stabilize the luminescent signal. The plates were analyzed with a Microplate Reader.

ADC direct killing was assessed in NCI-1650, Capan-1, and MDA-MB-453 cancer lines. Cells were seeded (NCI-1650 or MDA-MB-453 (2E3/well) or Capan-1 (4E3/well)) into 3D 96-well plates (Corning: 4520), 80 μl/well, and incubated at 37° C., 5% CO₂, overnight. Fresh growth medium was added containing varying concentrations of ADCs, 40 μl/well, and incubated at 37° C., 5% CO₂, for 6 days. The cell viability was detected by 3D reagent (Promega, G9683), 100 μl/well. The 3D plates were allowed to incubate at room temperature for 30 minutes to stabilize the luminescent signal. The plates were analyzed with a Microplate Reader.

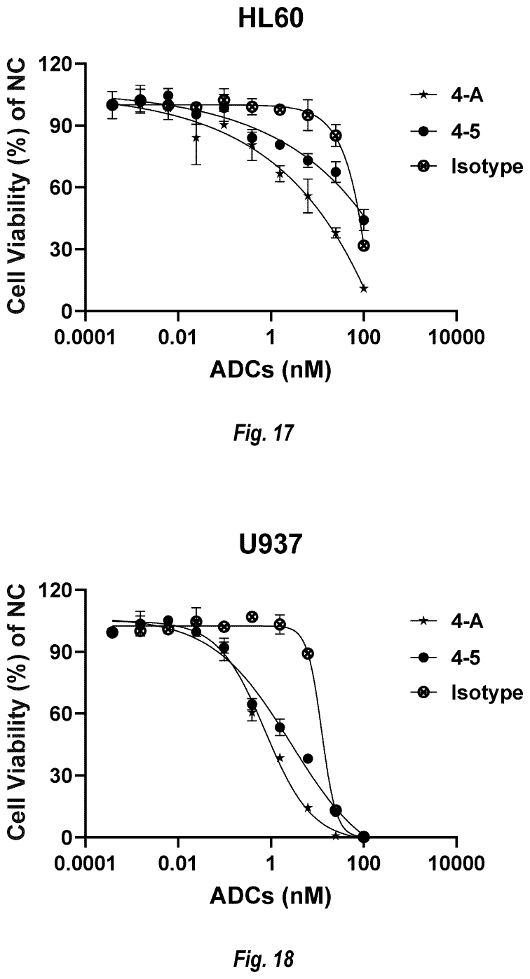

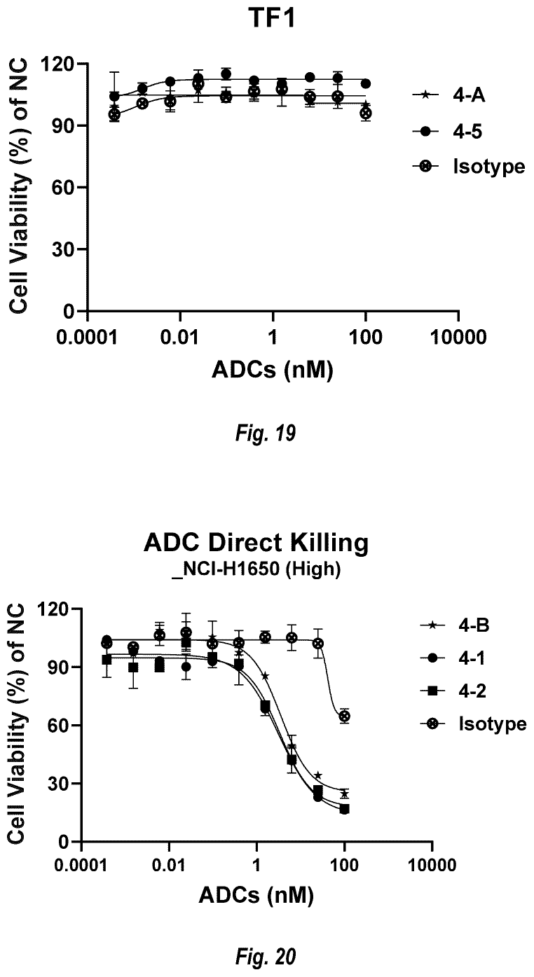

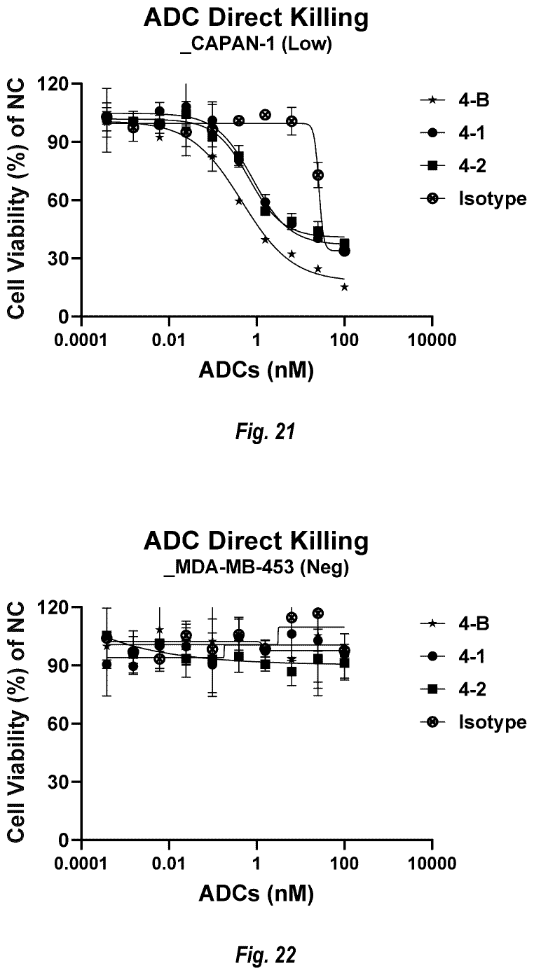

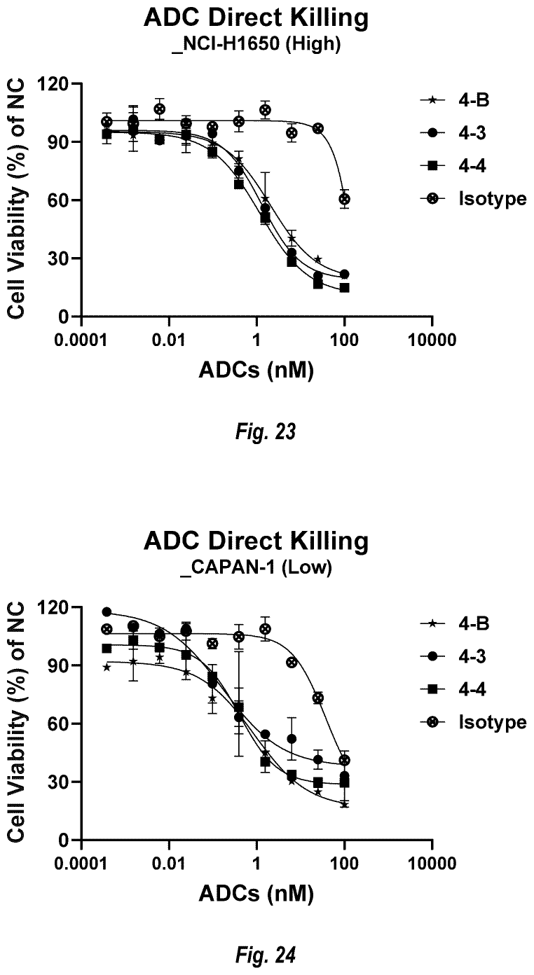

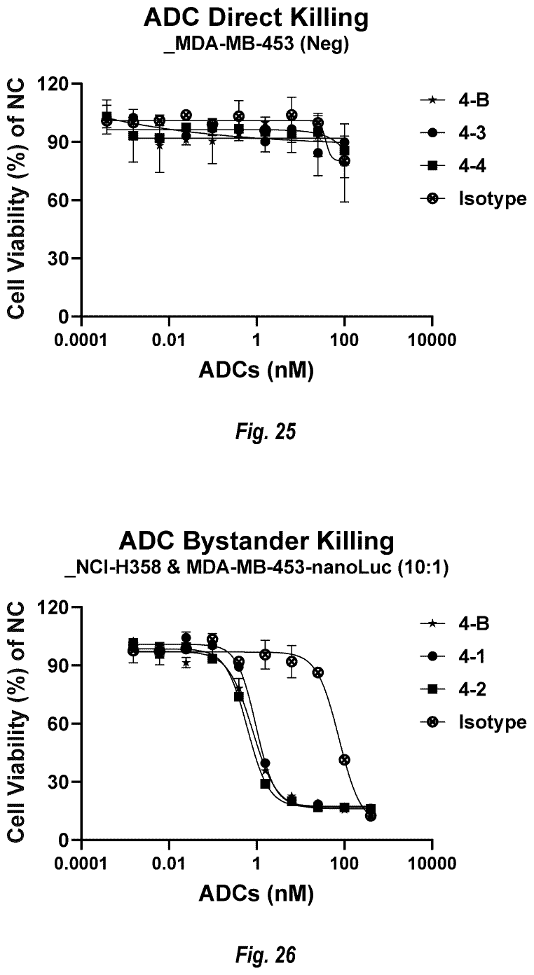

Data are summarized in FIGS. 17-25 and Tables 11-13.

TABLE 11
ADC direct killing potency

| | |
|---|---|
| U937 | HL60 |

| | Emax | EC50 | Emax | EC50 |
|---|---|---|---|---|
| ADC | (%) | (nM) | (%) | (nM) |
| 4-A | 99.9 | 0.8 | 89.0 | 6.3 |
| 4-5 | 99.5 | 0.2 | 55.9 | 8.2 |

TABLE 12
ADC direct killing potency on B7H3
high- and low-expression cell lines

| | NCI-H1650 (High), 3D | | CAPAN-1 (Low), 3D | |
|---|---|---|---|---|
| ADCs | Emax (%) | EC50 (nM) | Emax (%) | EC50 (nM) |
| 4-B | 75.3 | 3.9 | 84.9 | 0.5 |
| 4-1 | 83.6 | 2.8 | 66.4 | 1.1 |
| 4-2 | 83.0 | 2.9 | 62.8 | 0.8 |

TABLE 13
ADC direct killing potency on B7H3
high- and low-expression cell lines

| | NCI-H1650 (High), 3D | | CAPAN-1 (Low), 3D | |
|---|---|---|---|---|
| ADCs | Emax (%) | EC50 (nM) | Emax (%) | EC50 (nM) |
| 4-B | 78.9 | 1.7 | 81.7 | 0.4 |
| 4-3 | 78.1 | 1.1 | 66.9 | 0.4 |
| 4-4 | 85.2 | 0.9 | 70.4 | 0.4 |

ADC Bystander Killing in NCI-H358 Co-Culture with MDA-MB-453-nanoLuc

Method: MDA-MB-453-nanoLuc cell line construction. PT67-nanoLuc cells were cultured, then the cell-culture medium (containing the virus (nano-Luc gene)) was collected and filtered. MDA-MB-453 cells were seeded in 6-well plates at 1E5 cells/well, and incubated at 37° C., 5% CO₂, overnight. The PT67-nanoLuc cell medium and 8 μg/ml polybrene were added. The infection was repeated 3 times, every one day. Then the MDA-MB-453-nanoLuc cells were cultured with the addition of 1 mg/ml Geneticin for 5 days. The MDA-MB-453-nanoLuc cells were collected, and Nano-Glo reagent (Promega: N1120) was added to test the nano-Luc transfection efficiency.

Method: ADC bystander killing. NCI-H358 & MDA-MB-453-nanoLuc (10:1), or MDA-MB-453-nanoLuc cells alone were seeded into 3D-96-well plates (Corning: 4520) at 80 μl/well, and incubated at 37° C., 5% CO₂, overnight. Fresh growth medium containing the varying concentrations of ADCs was added at 40 μl/well. The cells were incubated at 37° C., 5% CO₂, 6 days. The 3D-plates were centrifuged at 1500 rpm, 25° C., 5 min, then the supernatant was discarded.

The Calu-6-nanoLuc cell viability was detected by Nano-Glo reagent (Promega: N1120), 150 μl/well. The 3D-plates were allowed to incubate at room temperature for 10 minutes to stabilize the luminescent signal. Then the plates were analyzed with a microplate reader.

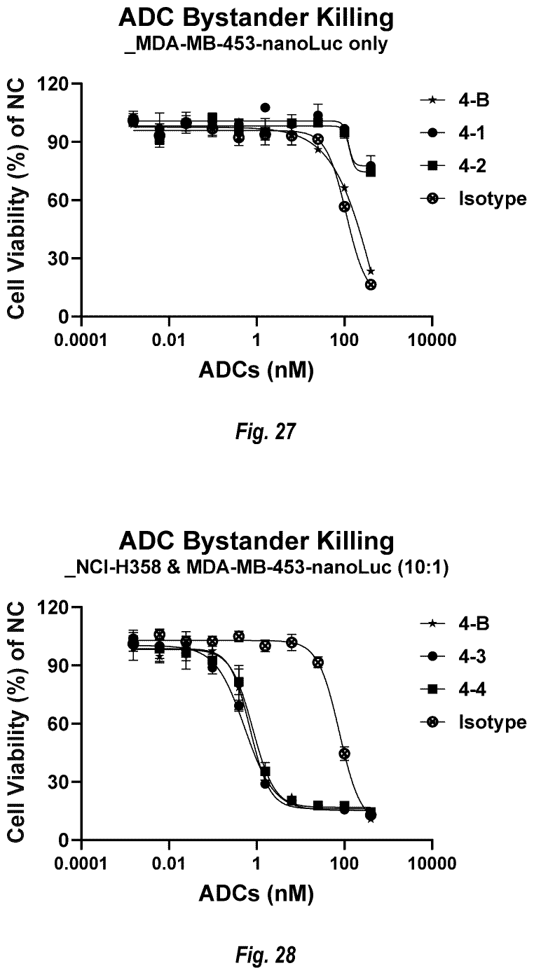

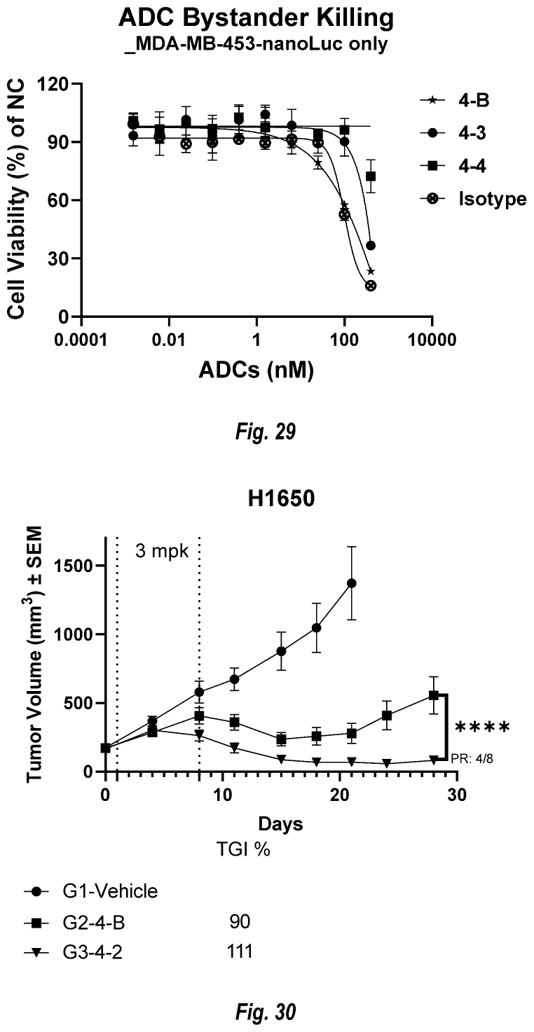

Results are presented in Tables 14-15 and FIGS. 26-29.

TABLE 14
ADC bystander killing effect on NCI-
H358/MDA-MB-453 (nano-Luc) co-culture

NCI-H358:MDA-MB-453-nanoLuc = 10:1, 3D

| ADC No. | Emax (%) | EC50 (nM) |
|---|---|---|
| 4-B | 88.1 | 0.8 |
| 4-1 | 83.7 | 1.0 |
| 4-2 | 83.8 | 0.6 |

TABLE 15
ADC bystander killing effect on NCI-
H358/MDA-MB-453 (nano-Luc) co-culture

NCI-H358:MDA-MB-453-nanoLuc = 10:1, 3D

| ADC No. | Emax (%) | EC50 (nM) |
|---|---|---|
| 4-B | 89.2 | 0.7 |
| 4-3 | 86.9 | 0.6 |
| 4-4 | 85.6 | 0.8 |

### ADC In Vivo Efficacy Study in H1650 Xenograft Model

Female BALB/c Nude mice were subcutaneously implanted with 3×10⁶ H1650 cells per 200 L PBS/matrigel in the right flank. After inoculation, tumor volumes were determined twice weekly in two dimensions using a caliper and were expressed in mm³ using the formula V=0.5(a×b²) where a and b are the long and short diameters of the tumor, respectively. When tumors reached a mean volume of approximately 200 mm³ in size, mice were randomly allocated into 3 groups with 7 animals in each group, and were intravenously treated with vehicle, or ADC 4-B, 4-2, 4-3 or 4-4 at 1 mpk or 3 mpk QW*2. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data are presented as mean tumor volume±standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

$$placebo t = treated tumor volume at time t$$

$$placebo {t}_{0} = treated tumor volume at time 0$$

$$placebo t = placebo tumor volume at time t$$

$$placebo {t}_{0} = placebo tumor volume at time 0$$

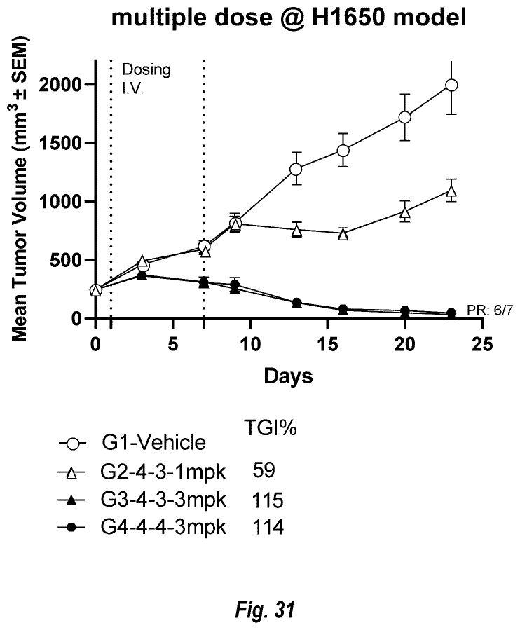

Results are presented in FIGS. 30 and 31. The results demonstrate that each of ADC 4-2, 4-3, and 4-4 showed good anti-tumor activity (FIGS. 30 and 31). ADC 4-2 exhibited better anti-tumor activity than did reference ADC 4-B (FIG. 30). ADC 4-3 and 4-4 exhibited comparable anti-tumor activity (FIG. 31). ADC 4-3 showed dose-dependent efficacy (FIG. 31).

### ADC Plasma Stability

Incubation of ADC with plasma: ADCs were diluted into mouse or human plasma to yield a final solution of 100 g/mL ADC in plasma. The samples were incubated at 37° C. Aliquots (100 μL) are taken at five time points (0, 2 (4), 24, 72, or 168 h). Samples were frozen at −80° C. until analysis.

Plasma payload concentrations were carried out under the following measurement conditions:

Instrument: LC-MS/MS (Triple Quad 6500 plus)

Monitor: MRM

Column: Advanced Materials Technology, HALO AQ-C18 2.7 μm 90 Å, 50*2.1 mm

Column temperature: 40° C.

Mobile phase A: H₂O-0.1% FA

Mobile phase B: ACN-0.11% FA

Gradient program for MMAE: 15% B-15% B (0 min-0.4 min), 15% B-30% B (0.4 min-0.8 min), 30% B-30% B (0.8 min-1.8 min), 30% B-90% B (1.8 min-1.9 min), 90% B-90% B (1.9 min-2.4 min), 90% B-15% B (2.4 min-2.5 min), 15% B-15% B (2.5 min-3.0 min)

Gradient program for Dxd: 2% B-2% B (0 min-0.2 min), 2% B-98% B (0.2 min-1.2 min), 98% B-98% B (1.2 min-2.0 min), 98% B-2% B (2.0 min-2.01 min), 2% B-2% B (2.01 min-4.0 min)

Injected sample amount: 10 μL (DXd or DXd analogues or MMAE)

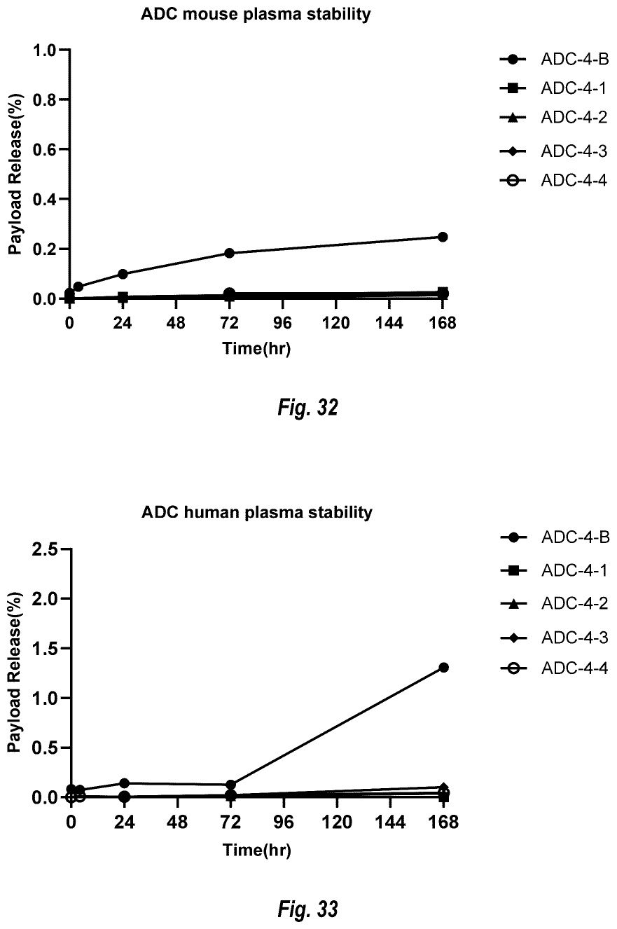

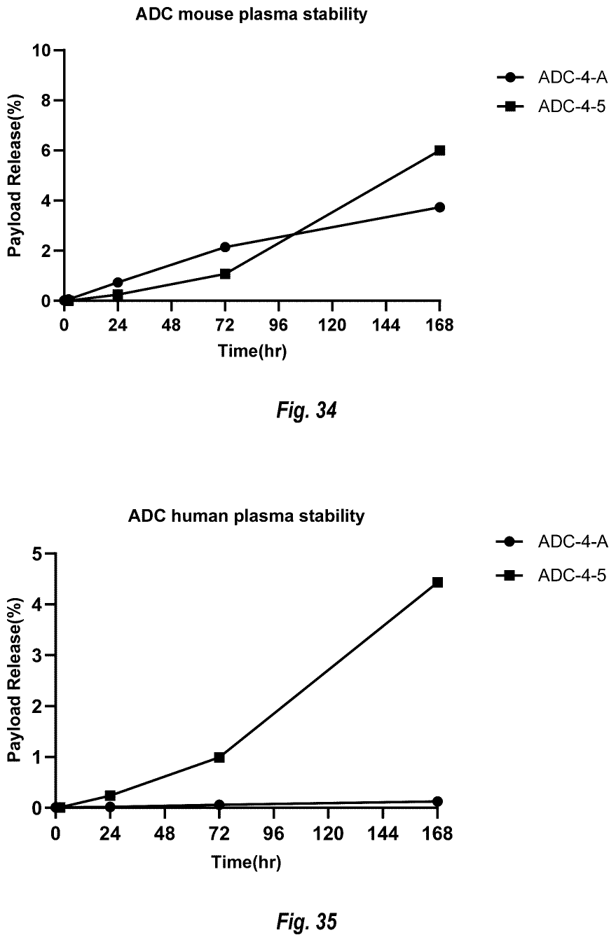

Results are presented in FIGS. 32-35. ADC 4-1 to 4-4 showed lower payload release % than did ADC-4-B in both mouse plasma (FIG. 32) and human plasma (FIG. 33). ADC 4-5 showed comparable payload release % to ADC 4-A in mouse plasma (FIG. 34) and higher payload release % to ADC 4-A in human plasma (FIG. 35).

### In Mice PK Study 1

After one dose of intravenously administered ADC (3 mg/kg) to H1650 tumor-bearing mice, blood samples were collected 0.0833, 2, 24, 72, 120, and 168 h later, followed by centrifugation (4° C., 3000×g, 7 min) to separate plasma. The concentrations of ADCs were measured by in-house developed Meso Scale Discovery (MSD) ligand binding methods. Briefly, a His-tagged B7H3 extracellular domain fusion protein was used as a capture reagent, and biotin-labelled anti-payload Ab was used as the detection reagent for ADCs. Plasma concentration of payload was measured using the same method as described above.

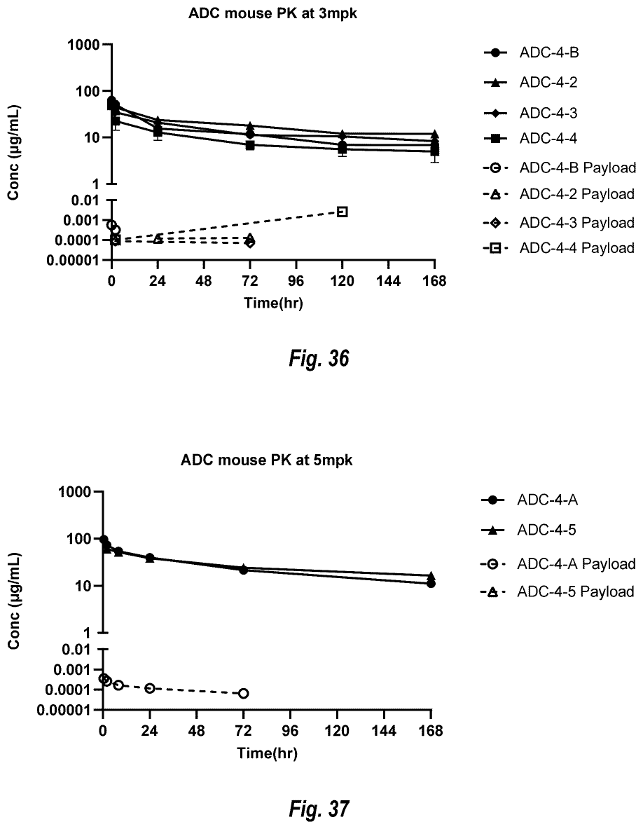

Results are presented in FIG. 36. The results demonstrate that ADC 4-2 to 4-4 showed comparable PK profiles to ADC 4-B in an H1650 efficacy model.

### In Mice PK Study 2

After one dose of intravenously administered ADC (5 mg/kg) to non-tumor-bearing mice, blood samples were collected 0.5, 2, 8, 24, 72 and 168 h later, followed by centrifugation (4° C., 3000×g, 7 min) to separate plasma. The concentrations of ADCs were measured by in-house developed Meso Scale Discovery (MSD) ligand binding methods. Briefly, a His-tagged CLL1 extracellular domain fusion protein was used as a capture reagent, and biotin-labelled anti-payload Ab was used as the detection reagent for ADCs. Plasma concentration of payload was measured using the same method as described above.

Results are presented in FIG. 37. The results demonstrate that ADC 4-5 showed comparable PK profiles to ADC 4-A in non-tumor-bearing mice.

Although the foregoing disclosure has been presented in some detail by way of illustration and example for purposes of clarity of understanding, it is apparent to those skilled in the art that certain minor changes and modifications will be practiced. Therefore, the description and examples should not be construed as limiting.

It is to be understood that, if any publication is referred to herein, such reference does not constitute an admission that the publication forms a part of the common general knowledge in the art in any country.

The disclosures of all non-patent publications, patents, patent applications, and published patent applications referred to herein are hereby incorporated herein by reference in their entireties.

What is claimed is:

1. A compound of Formula (I): 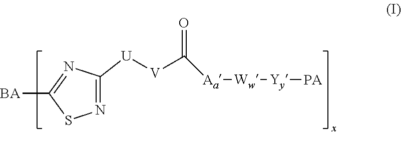
or a pharmaceutically acceptable salt, tautomer, solvate, or stereoisomer thereof, wherein:
BA is a binding agent selected from a humanized, chimeric, or human antibody or an antigen binding fragment thereof,
U is arylene, heteroarylene, or a bond;
V is a bond or —C≡C—(CH₂)ₙ—;
n is an integer between 0 and 10;
A is a Stretcher unit residue;
subscript a′ is 0 or 1;
W is a Cleavable unit;
subscript w′ is 0 or 1;
Y is a Spacer unit;
subscript y′ is 0 or 1;
PA is a payload residue; and
subscript x is from 1 to 15.

2. The compound of claim 1, wherein U is arylene.

3. The compound of claim 2, wherein U is phenylene.

4. The compound of claim 3, wherein U is

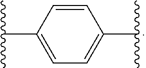 [SMILES: *c1ccc(*)cc1]

5. The compound of any one of claims 1-4, wherein V is a bond.

6. The compound of any one of claims 1-4, wherein V is —C≡C—(CH₂)ₙ—.

7. The compound of claim 6, wherein V is —C≡C—(CH₂)₃—.

8. The compound of claim 1, wherein U is heteroarylene.

9. The compound of claim 8, wherein U is a bivalent pyrimidine ring.

10. The compound of claim 9, wherein U is

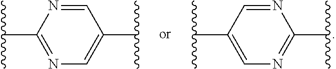 [SMILES: *c1cnc(*)nc1 ; *c1cnc(*)nc1]

11. The compound of any one of claims 8-10, wherein V is a bond.

12. The compound of any one of claims 8-10, wherein V is —C≡C—(CH₂)ₙ—.

13. The compound of claim 12, wherein V is —C≡C—(CH₂)₃—.

14. The compound of claim 1, wherein U is a bond.

15. The compound of claim 14, wherein V is —C≡C—(CH₂)ₙ—.

16. The compound of claim 15, wherein V is —C≡C—(CH₂)₃—.

17. The compound of any one of claims 1-16, wherein A is —(CH₂)ₘ—C(═O)—, —CH₂—C(═O)—NH—(CH₂)ₘ—C(═O)—, —(CH₂CH₂O)ₘ—CH₂CH₂—C(═O)—, —CH[—(CH₂)ₘ—COOH]—C(═O)—, —CH₂—C(═O)—NH—(CH₂)ₘ—C(═O)—NH—(CH₂)ₘ—C(═O)—, —C(═O)—(CH₂)ₘ—C(═O)—, —NH—(CH₂)ₘ—C(═O)—, or —NH—(CH₂CH₂O)ₘ—CH₂CH₂—C(═O)—, and each m independently represents an integer of 1, 2, 3, 4, or 5.

18. The compound of any one of claims 1-17, wherein subscript a′ is 0.

19. The compound of any one of claims 1-17, wherein subscript a′ is 1.

20. The compound of any one of claims 1-19, wherein W is one of the following formulas: 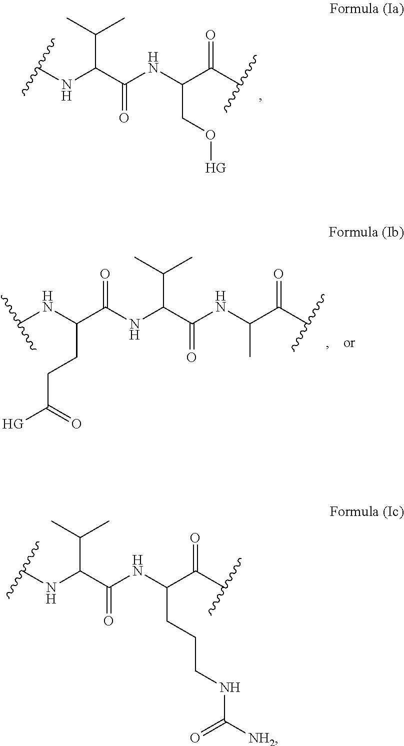
wherein HG is a hydrophilic moiety or hydrogen.

21. The compound of claim 20, wherein HG is a saccharide, phosphate ester, sulfate ester, phosphodiester, or phosphonate.

22. The compound of claim 21, wherein HG is a saccharide and the saccharide is β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid, sulfate, phosphate, carboxyl, amino, or O— acetyl modification thereof.

23. The compound of claim 20, wherein HG is

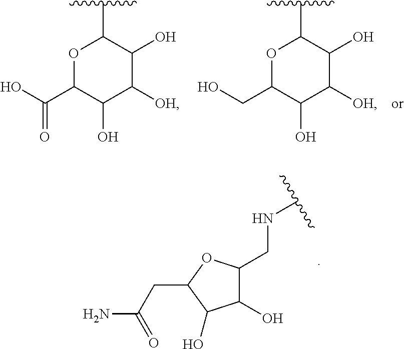 [SMILES: *C1OC(C(=O)O)C(O)C(O)C1O ; *C1OC(CO)C(O)C(O)C1O ; *NCC1OC(CC(N)=O)C(O)C1O]

24. The compound of any one of claims 1-23, wherein subscript w′ is 1.

25. The compound of any one of claims 1-23, wherein subscript w′ is 0.

26. The compound of any one of claims 1-23, wherein Y is —NH—CH₂—O— or —NH-(p-C₆H₄)—CH₂—O—.

27. The compound of any one of claims 1-26, wherein subscript y′ is 1.

28. The compound of any one of claims 1-26, wherein subscript y′ is 0.

29. The compound of any one of claims 1-248, wherein the compound is one of the following formulas:

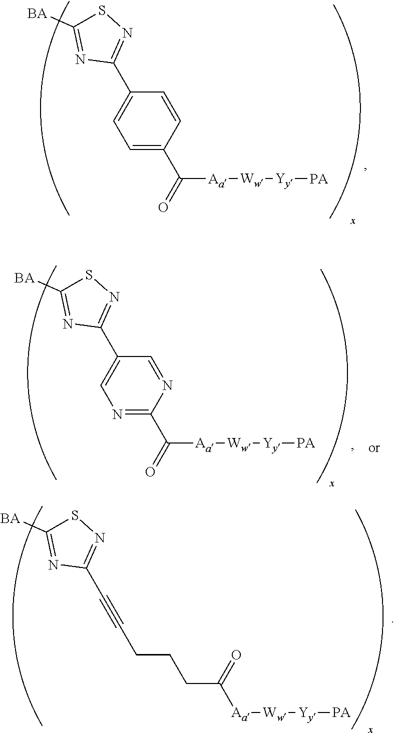

30. The compound of any one of claims 1-16 and 29, wherein the fraction 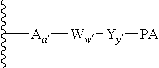
is one of the following formulas:

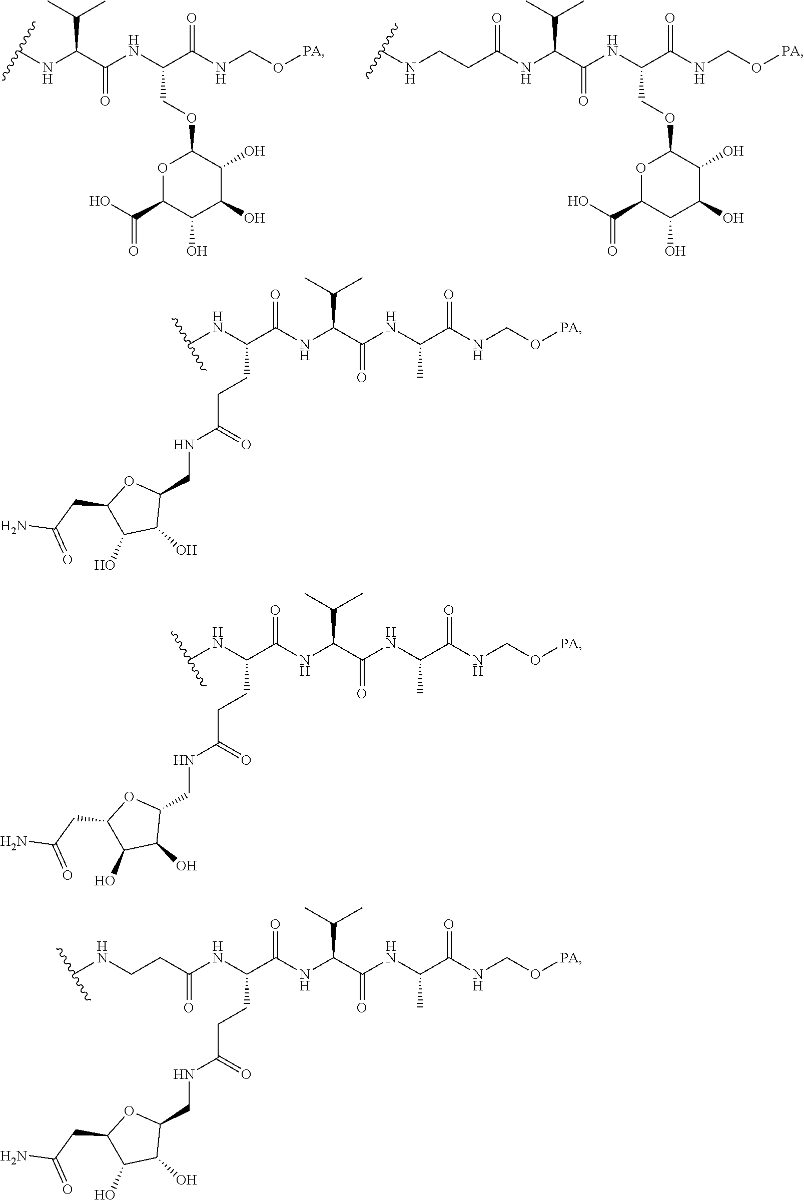

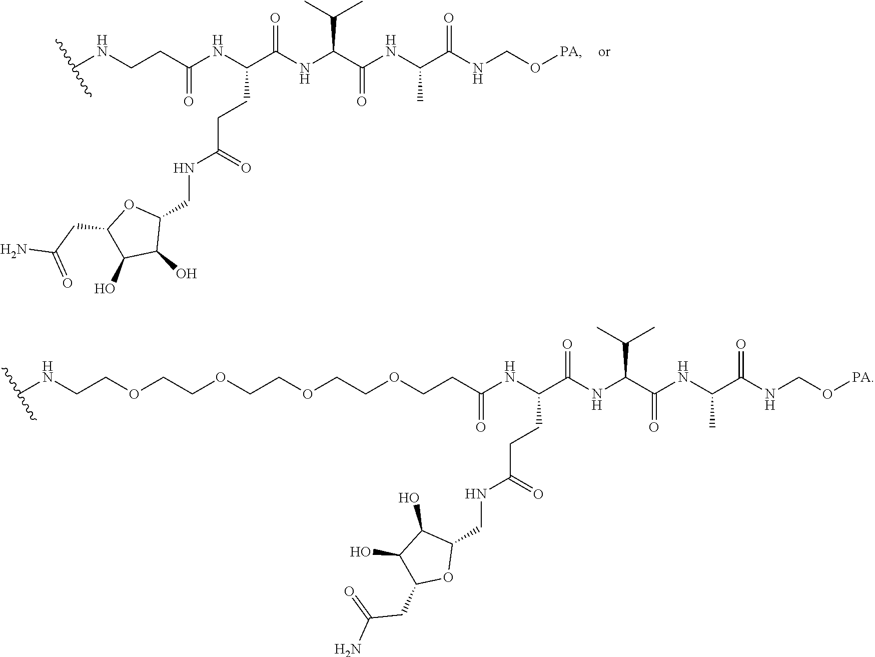

31. The compound of any one of claims 1-30, wherein BA is ifinatamab, 6E7, or trastuzumab, or an antigen binding fragment of ifinatamab, 6E7, or trastuzumab.

32. The compound of any one of claims 1-30, wherein BA is a humanized, chimeric, or human antibody or the antigen binding fragment thereof which binds to one or more of receptors chosen from HER2, CLL1, or B7H3.

33. The compound of claim 1, wherein the compound is 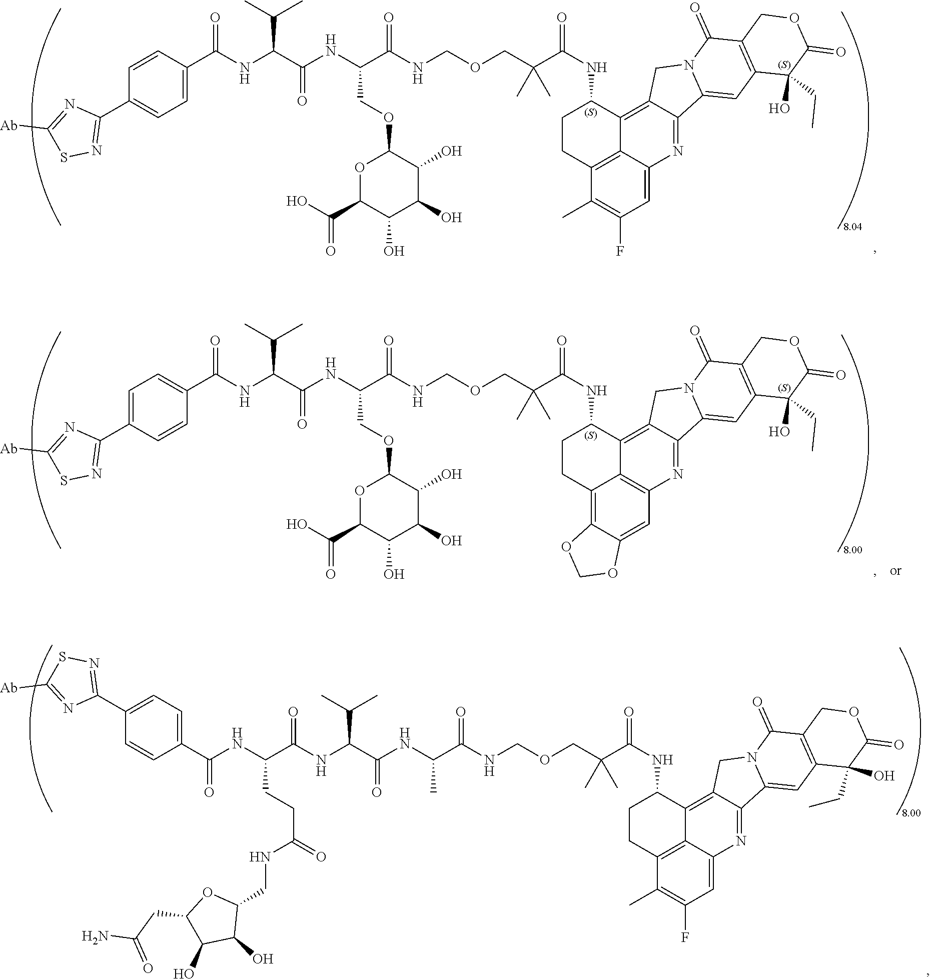
or a pharmaceutically acceptable salt, tautomer, or solvate thereof, wherein Ab is ifinatamab.

34. The compound of claim 1, wherein the compound is 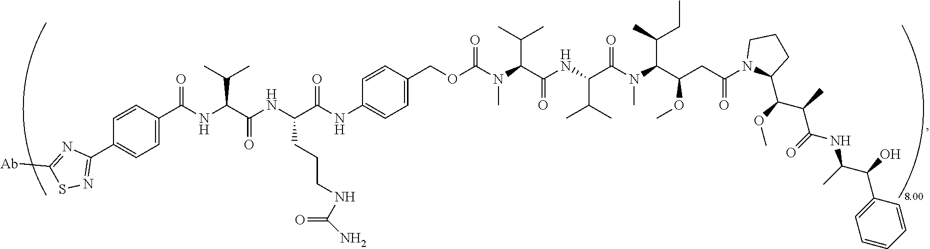
or a pharmaceutically acceptable salt, tautomer, or solvate thereof, wherein Ab is 6E7.

35. A pharmaceutical composition comprising a compound of any one of claims 1-34, or a pharmaceutically acceptable salt, tautomer, solvate, or stereoisomer thereof, and a pharmaceutically acceptable excipient.

36. A compound of Formula (II): 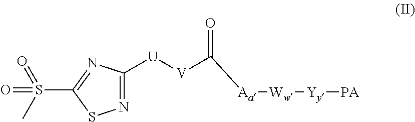
or a pharmaceutically acceptable salt, tautomer, solvate, or stereoisomer thereof, wherein:
U is arylene, heteroarylene, or a bond;
V is a bond or —C≡C—(CH₂)ₙ—;
n is an integer between 0 and 10;
A is a Stretcher unit residue;
subscript a′ is 0 or 1;
W is a Cleavable unit;
subscript w′ is 0 or 1;
Y is a Spacer unit;
subscript y′ is 0 or 1; and
PA is a payload residue.

37. The compound of claim 36, wherein U is arylene.

38. The compound of claim 37, wherein U is phenylene.

39. The compound of claim 38, wherein U is

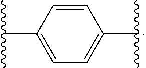 [SMILES: *c1ccc(*)cc1]

40. The compound of any one of claims 36-39, wherein V is a bond.

41. The compound of any one of claims 36-39, wherein V is —C≡C—(CH₂)ₙ—.

42. The compound of claim 41, wherein V is —C≡C—(CH₂)₃—.

43. The compound of claim 36, wherein U is heteroarylene.

44. The compound of claim 43, wherein U is a bivalent pyrimidine ring.

45. The compound of claim 44, wherein U is

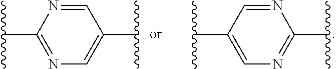 [SMILES: *c1cnc(*)nc1 ; *c1cnc(*)nc1]

46. The compound of any one of claims 43-45, wherein V is a bond.

47. The compound of any one of claims 43-45, wherein V is —C≡C—(CH₂)ₙ—.

48. The compound of claim 47, wherein V is —C≡C—(CH₂)₃—.

49. The compound of claim 36, wherein U is a bond.

50. The compound of claim 49, wherein V is —C≡C—(CH₂)ₙ—.

51. The compound of claim 50, wherein V is —C≡C—(CH₂)₃—.

52. The compound of any one of claims 36-51, wherein A is —(CH₂)ₘ—C(═O)—, —CH₂—C(═O)—NH—(CH₂)ₘ—C(═O)—, —(CH₂CH₂O)ₘ—CH₂CH₂—C(═O)—, —CH[—(CH₂)ₘ—COOH]—C(═O)—, —CH₂—C(═O)—NH—(CH₂)ₘ—C(═O)—NH—(CH₂)ₘ—C(═O)—, —C(═O)—(CH₂)ₘ—C(═O)—, —NH—(CH₂)ₘ—C(═O)—, or —NH—(CH₂CH₂O)ₘ—CH₂CH₂—C(═O)—, and each m independently represents an integer of 1, 2, 3, 4, or 5.

53. The compound of any one of claims 36-52, wherein subscript a′ is 0.

54. The compound of any one of claims 36-52, wherein subscript a′ is 1.

55. The compound of any one of claims 36-54, wherein W is one of the following formulas: 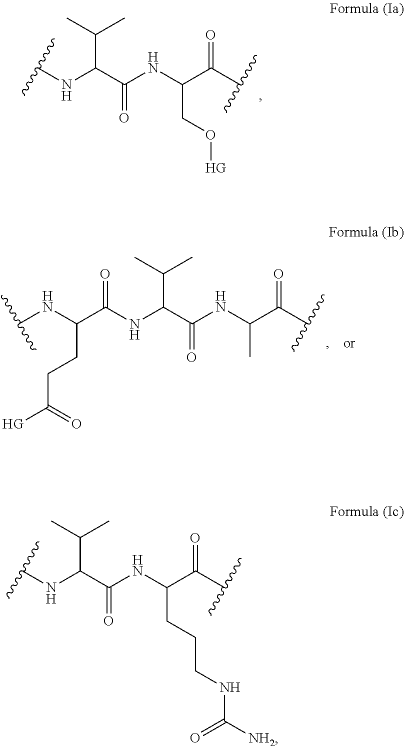
and wherein HG is a hydrophilic moiety or hydrogen.

56. The compound of claim 55, wherein HG is a saccharide, phosphate ester, sulfate ester, phosphodiester, or phosphonate.

57. The compound of claim 56, wherein HG is a saccharide and the saccharide is β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid, sulfate, phosphate, carboxyl, amino, or O— acetyl modification thereof.

58. The compound of claim 55, wherein HG is

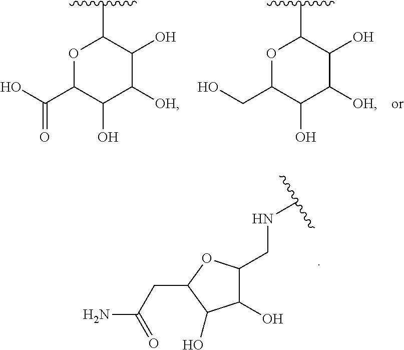 [SMILES: *C1OC(C(=O)O)C(O)C(O)C1O ; *C1OC(CO)C(O)C(O)C1O ; *NCC1OC(CC(N)=O)C(O)C1O]

59. The compound of any one of claims 36-58, wherein subscript w′ is 1.

60. The compound of any one of claims 36-58, wherein subscript w′ is 0.

61. The compound of any one of claims 36-60, wherein Y is —NH—CH₂—O— or —NH-(p-C₆H₄)—CH₂—O—.

62. The compound of any one of claims 36-61, wherein subscript y′ is 1.

63. The compound of any one of claims 36-61, wherein subscript y′ is 0.

64. The compound of any one of claims 36-63, wherein the compound is one of the following formulas:

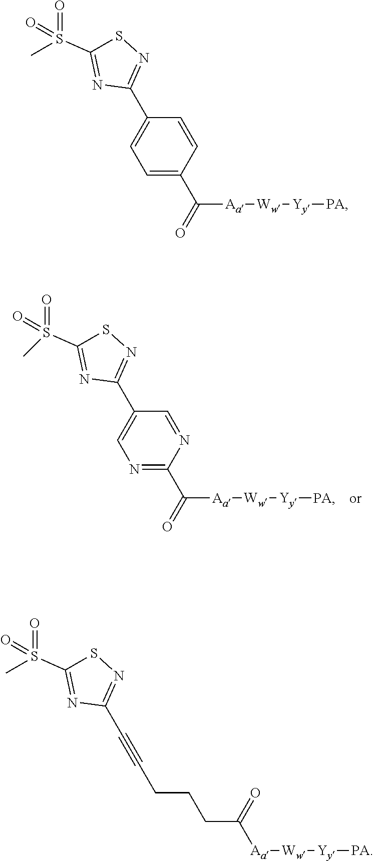

65. The compound of any one of claims 36-51 and 64, wherein the fraction 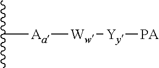
is one of the following formulas:

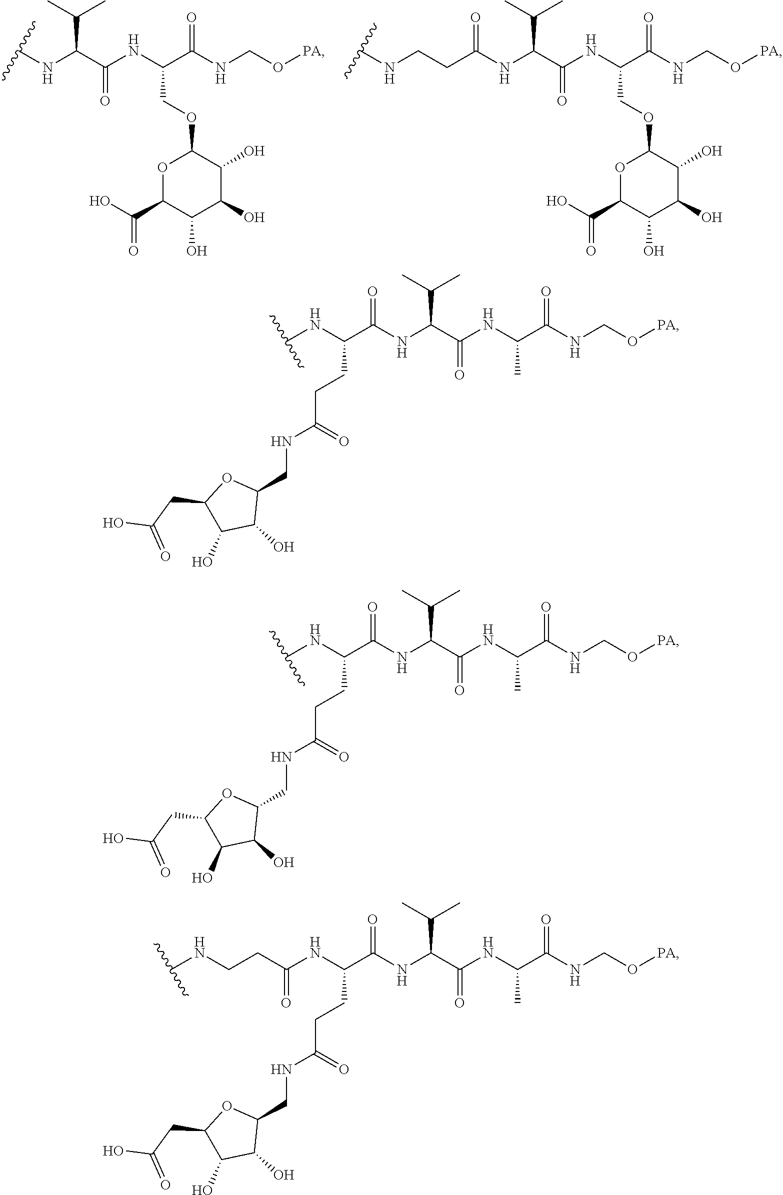

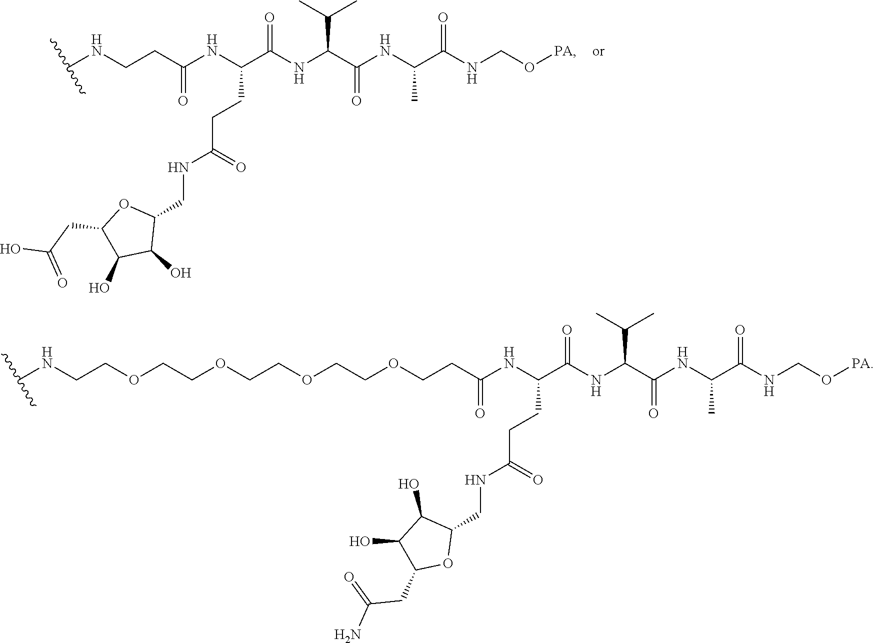

66. The compound of claim 65, wherein the compound is 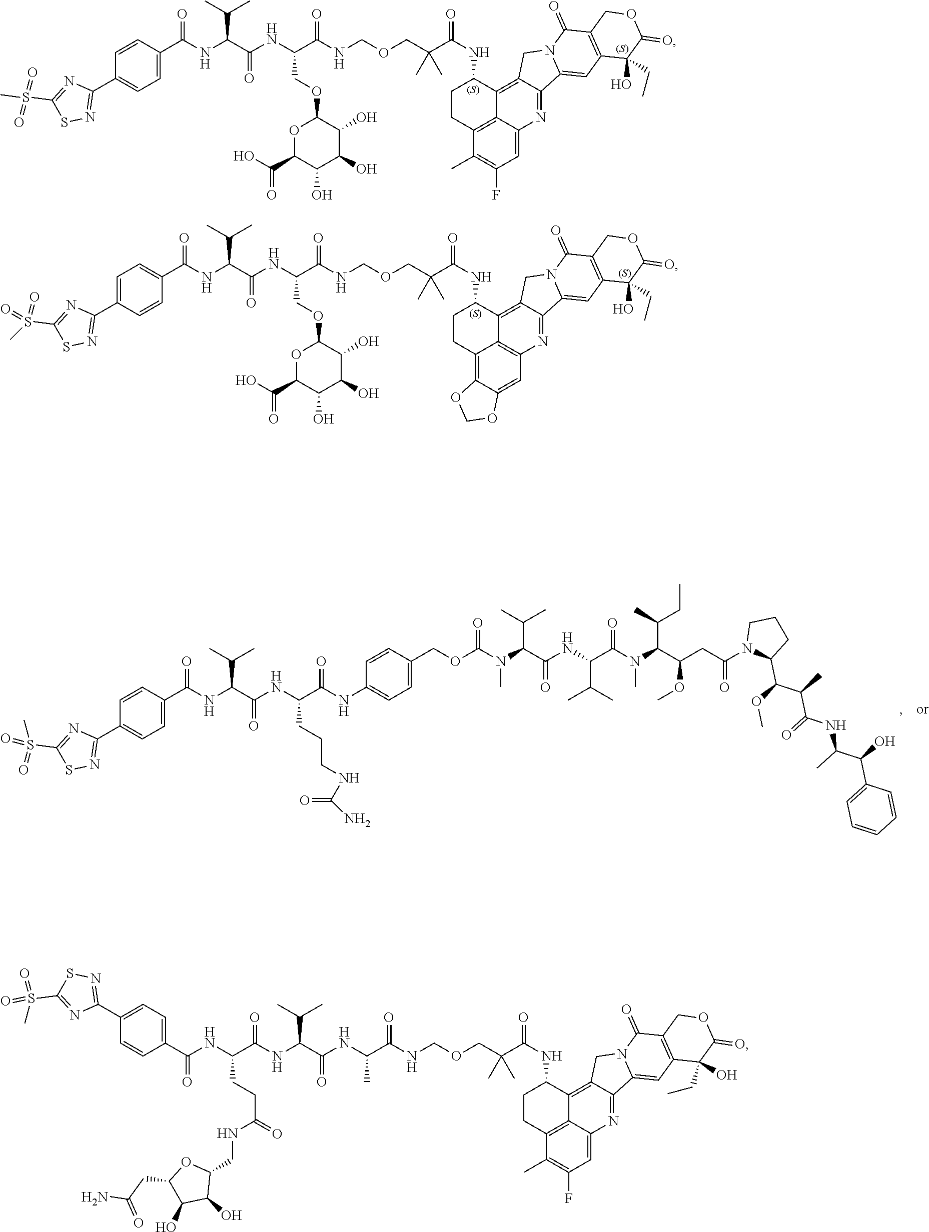
or a pharmaceutically acceptable salt, tautomer, or solvate thereof.

67. The compound of any one of claims 1-32 or 36-65, wherein each PA is independently a cytotoxic agent.

68. The compound of claim 67, wherein each PA is independently selected from a residue of the group consisting of DXd, 7-ethyl-10-hydroxy-camptothecin (SN-38), and monomethyl auristatin E (MMAE).

69. The compound of claim 68, wherein each PA independently is a compound of formula (VI): 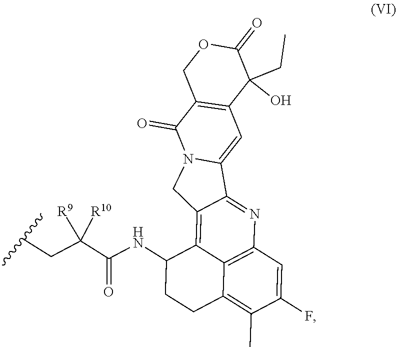
wherein each of R⁹ and R¹⁰ is independently hydrogen, halogen, or substituted or unsubstituted C₁₋₄ alkyl.

70. The compound of claim 69, wherein each PA is independently

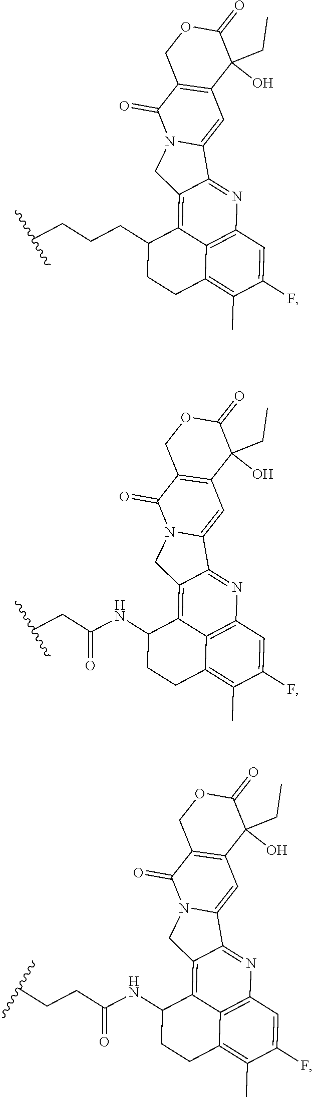 [SMILES: *CCCC1CCc2c(C)c(F)cc3nc4c(c1c23)Cn1c-4cc2c(c1=O)COC(=O)C2(O)CC ; *CC(=O)NC1CCc2c(C)c(F)cc3nc4c(c1c23)Cn1c-4cc2c(c1=O)COC(=O)C2(O)CC ; *CCC(=O)NC1CCc2c(C)c(F)cc3nc4c(c1c23)Cn1c-4cc2c(c1=O)COC(=O)C2(O)CC]

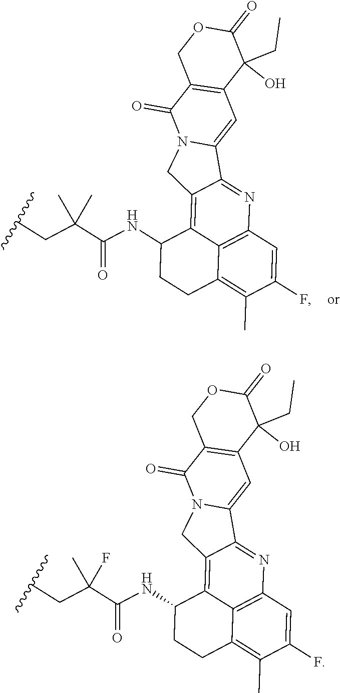

71. A compound of Formula (III): 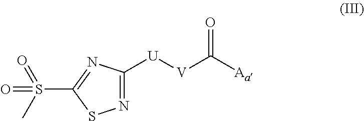
or a pharmaceutically acceptable salt, tautomer, solvate, or stereoisomer thereof, wherein:
U is arylene, heteroarylene, or a bond;
V is a bond or —C≡C—(CH₂)ₙ—;
n is an integer between 0 and 10;
A is a Stretcher unit; and
subscript a′ is 0 or 1.

72. The compound of claim 71, wherein U is arylene.

73. The compound of claim 72, wherein U is phenylene.

74. The compound of claim 73, wherein U is

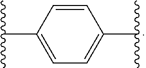 [SMILES: *c1ccc(*)cc1]

75. The compound of any one of claims 71-74, wherein V is a bond.

76. The compound of any one of claims 71-74, wherein V is —C≡C—(CH₂)ₙ—.

77. The compound of claim 76, wherein V is —C≡C—(CH₂)₃—.

78. The compound of claim 71, wherein U is heteroarylene.

79. The compound of claim 78, wherein U is a bivalent pyrimidine ring.

80. The compound of claim 79, wherein U is

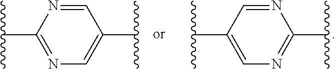 [SMILES: *c1cnc(*)nc1 ; *c1cnc(*)nc1]

81. The compound of any one of claims 78-80, wherein V is a bond.

82. The compound of any one of claims 78-80, wherein V is —C≡C—(CH₂)ₙ—.

83. The compound of claim 82, wherein V is —C≡C—(CH₂)₃—.

84. The compound of claim 71, wherein U is a bond.

85. The compound of claim 84, wherein V is —C≡C—(CH₂)ₙ—.

86. The compound of claim 85, wherein V is —C≡C—(CH₂)₃—.

87. The compound of any one of claims 71-86, wherein:
A is a bond, —OH, —CH₃, —N(CH₃)₂, —(CH₂)ₘ—C(═O)R⁷, —CH₂—C(═O)—NH—(CH₂)ₘ—C(═O)R⁷, —(CH₂CH₂O)ₘ—CH₂CH₂—C(═O)R⁷, —CH[—(CH₂)ₘ—COOH]—C(═O)R⁷, —CH₂—C(═O)—NH—(CH₂)ₘ—C(═O)—NH—(CH₂)ₘ—C(═O)R⁷, —C(═O)—(CH₂)ₘ—C(═O)R⁷, —NH—(CH₂)ₘ—C(═O)R⁷, or —NH—(CH₂CH₂O)ₘ—CH₂CH₂—C(═O)R⁷;
each m independently represents an integer of 1, 2, 3, 4, or 5;
R⁷ is OH or NR^{8a}R^{8b}; and
each of R^{8a} and R^{8b} is, independently, H; substituted or unsubstituted C₁₋₄ alkyl; substituted or unsubstituted C₃₋₅ cycloalkyl; or R^{8a} and R^{8b} together with the atom to which they are attached form a substituted or unsubstituted C₃₋₅ cycloalkyl.

88. The compound of claim 87, wherein R⁷ is OH, NH₂, NHCH₃, or N(CH₃)₂.

89. The compound of any one of claims 71-88, wherein subscript a′ is 0.

90. The compound of any one of claims 71-88, wherein subscript a′ is 1.

91. The compound of claim 88, wherein the compound is 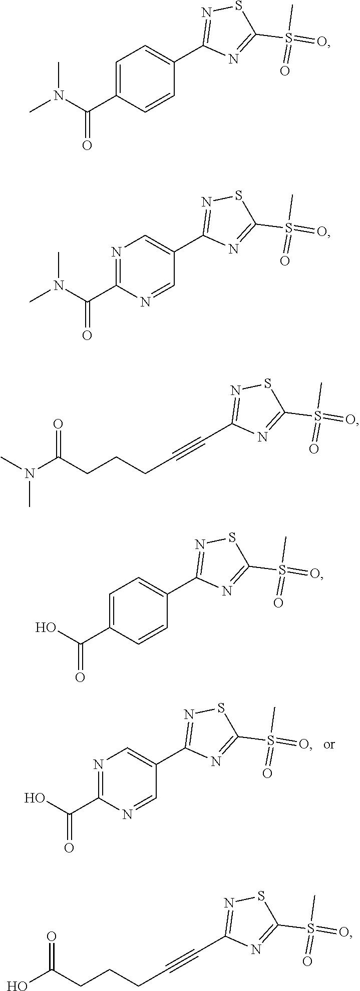
or a pharmaceutically acceptable salt, tautomer, or solvate thereof.
